(19) United States
(12) Patent Application Publication
(10) Pub. No.: US 2026/0227398 A1
(43) Pub. Date: Aug. 6, 2026

(54) CD62L (L-SELECTIN) APTAMER AND ITS APPLICATION FOR T CELL ISOLATION

(71) Applicants: University of Washington, Seattle, WA (US); Seattle Children's Hospital d/b/a Seattle Children's Research Institute, Seattle, WA (US)

(72) Inventors: Suzie Hwang Pun, Seattle, WA (US); Emmeline Cheng, Seattle, WA (US); Nataly Kacherovsky, Seattle, WA (US); Yuan-Che Wu, Seattle, WA (US); Michael C. Jensen, Bainbridge Island, WA (US)

(73) Assignees: University of Washington, Seattle, WA (US); Seattle Children's Hospital d/b/a Seattle Children's Research Institute, Seattle, WA (US)

(21) Appl. No.: 19/150,516

(22) PCT Filed: Jan. 23, 2024
(86) PCT No.: PCT/US2024/012651

§ 371 Date: Jul. 23, 2025

Related U.S. Application Data
(60) Provisional application No. 63/441,083, filed on Jan. 25, 2023.

(51) Int. Cl. G01N 33/569; C12N 5/00; C12N 15/115; G01N 33/68

(52) U.S. Cl. CPC G01N 33/56972; C12N 5/0087; C12N 15/115; G01N 33/6872; C12N 2310/16; G01N 2333/70564

(57) ABSTRACT

Various implementations described herein relate to oligonucleotides that specifically bind L-selectin. According to some implementations, oligonucleotides are conjugated to a support, a tag, a linker, or a drug. An example method includes exposing a solution of cells to the oligonucleotides and isolating cells that express L-selectin from the solution of cells, wherein the cells that express L-selectin are bound to the oligonucleotides. The example method further includes applying a reversal construct to release the cells that express L-selectin from the oligonucleotides. Example methods and compositions described herein can be used for cell selection, diagnostic, therapeutic, or research purposes.

### CROSS-REFERENCE TO RELATED APPLICATION(S)

This application is a U.S. National Stage which claims priority of International PCT Application No. PCT/US2024/012651, filed on Jan. 23, 2024, which claims the priority of U.S. Provisional App. No. 63/441,083, filed on Jan. 25, 2023, which are incorporated by reference herein in their entirety.

### REFERENCE TO SEQUENCE LISTING

The Sequence Listing associated with this application is provided in XML format in lieu of a paper copy and is hereby incorporated by reference into the specification. The name of the file containing the Sequence Listing is W149-0045US_Seq.xml. The file is 47,372 bytes, was created Jul. 23, 2025, and is being submitted electronically via Patent Center

### TECHNICAL FIELD

This application relates to oligonucleotides that specifically bind L-selectin. The oligonucleotides can be used for cell selection, diagnosis, research, and therapeutic purposes.

### BACKGROUND

T cells include a heterogeneous and diverse population of cells that contribute different immunological functions. Naïve T cells that leave the thymus with epitope specificity can proliferate and differentiate upon experiencing antigen to help clear the insult; over 95% of the resulting effector cells may then die while a small population of memory T cells remain for immune surveillance (Mahnke, Y. D., et al., Eur. J. Immunol., 2013. 43(11): p. 2797-2809). As naïve T cells (T_{N}) differentiate from stem cell memory T cells (T_{SCM}) to central memory T cells (T_{CM}), effector memory T cells (T_{EM}) and finally terminal effector T cells (T_{TE}), cells in general gain increased cytolytic capability while losing lymphoid homing and proliferation potential (Muroyama, Y. and E. J. Wherry, Cold Spring Harbor Perspect. Biol., 2021. 13(10): p. a037929.; Gattinoni, L., et al., Nat. Med., 2011. 17(10): p. 1290-1297).

Several studies have reported that T cell immunotherapies derived from less differentiated T_{N}, T_{SCM}, and T_{CM} are more potent than cell therapies manufactured from higher populations of differentiated cells. Adoptive transfer of antigen-specific T cells derived from T_{CM} but not T_{TE} populations show persistence in macaques (Berger, C., et al., J. Clin. Invest., 2008. 118(1): p. 294-305). The expansion of CAR T cell therapies in clinical studies correlates with the percentage of TSCM cells in the CAR T cell product (Arcangeli, S., et al., Front. Immunol., 2020. 11: p. 1217.; Xu, Y., et al., Blood, 2014. 123(24): p. 3750-3759). Sommermeyer and colleagues used fluorescence activated cell sorting (FACS) to isolate T_{N}, T_{CM} and T_{TE} subsets from human donors for CD19 CAR T cell production. Evaluation of the resulting CAR T cells in murine lymphoma model demonstrated that CAR T cells generated from less-differentiated T_{N} and T_{CM} cells had better anti-tumor efficacy than CAR T cells from T_{TE} cells (Sommermeyer, D., et al., Leukemia, 2016. 30(2): p. 492-500). Indeed, the fraction of CD8+T_{SCM} and T_{CM} cells in patients receiving CAR T cell therapies correlates with in vivo T cell expansion and clinical remission; this factor eclipses even disease or patient-related differences in prediction of a remission outcome (Xu, Y., et al., Blood, 2014. 123(24): p. 3750-3759; Fraietta, J. A., et al., Nat. Med., 2018. 24(5): p. 563-571).

One of the major markers for the T_{N}, T_{SCM}, and T_{CM} populations is L-selectin, also called CD62L. Selectins are a family of cell adhesion proteins with three members: E (“endothelial”)-Selectin, P (“platelet”)-Selectin, and L (“leukocyte”)-Selectin. Individual selectins have an N-terminal lectin domain that mediate cell binding to carbohydrate ligands, followed by an EGF-like motif, short consensus repeats and a transmembrane domain with a short cytoplasmic tail (Rosen, S. D. and C. R. Bertozzi, Curr. Opin. Cell Biol., 1994. 6(5): p. 663-673). As its name implies, CD62L/L-Selectin is expressed on hematopoietic cells, supports leukocyte extravasation from the vasculature, and contributes to the lymph node-homing characteristic of memory cells. CD62L is shed upon activation and not expressed on TEM and TTE populations. Clinical studies evaluating CAR T cells derived from naïve and memory T cell subsets have used CD62L as a selection marker during CAR T cell manufacturing (Sabatino, M., et al., Blood, 2016. 128(4): p. 519-528; Terakura, S., et al., Blood, 2012. 119(1): p. 72-82; Wang, X., et al., J. Immunother., 2012. 35(9): p. 689-701; Wang, X., et al., Blood, 2016. 127(24): p. 2980-2990). However, precise subset selection also increases manufacturing complexity, time, and cost and reduces product yield.

### BRIEF DESCRIPTION OF THE DRAWINGS

Some of the drawings submitted herewith may be better understood in color. Applicant considers the color versions of the drawings as part of the original submission and reserves the right to present color images of the drawings in later proceedings.

FIG. 1 illustrates an example environment for cell selection using an oligonucleotide that specifically binds L-selectin.

FIG. 2A illustrates an example oligonucleotide conjugated to a solid support.

FIG. 2B illustrates the example oligonucleotide conjugated to a particle.

FIG. 2C illustrates the example oligonucleotide conjugated to a tag.

FIG. 3 illustrates an example method for isolating a cell that expresses L-selectin using an oligonucleotide that specifically binds L-selectin

FIGS. 4A-4E illustrate cell-SELEX workflow and results. (4A) Schematic of cell-SELEX. An N52 DNA library underwent 7 rounds of cell-SELEX using primary human T cells for positive selection and J.RT3-T3.5 cells or T cell-depleted apheresis product for negative selection. In round 8, the library was incubated with primary human PBMCs, and T cells and bound sequences were isolated by negative magnetic selection. (4B) Flow cytometry analysis of binding of 250 nM round 8 library (red) or a random aptamer control (RAN) to CD4+ T cells (left) and T cell-depleted PBMCs (right) cultured in Roswell Park Memorial Institute (RPMI) Medium with 10% FBS. (4C) Binding curve of A1 to CD4+ T cells thawed after cryopreservation. (4D) Binding curve of A1 to CD4+ T cells cultured in RPMI medium with 10% FBS; (4E) Evaluation of selected aptamers from R8 library to human primary CD4+ T cells.

FIG. 5 illustrates the phylogenetic tree and predicted binding motifs generated using the top 50 sequences in the final round library. Circled aptamers were chosen for binding studies.

FIG. 6 illustrates results of the flow cytometry binding study on target cells by aptamer candidates selected from the round 8 library based on sequence analysis.

FIGS. 7A, 7B illustrate binding of 20 nM A1 (right) and random aptamer (RAN, left) to cryopreserved human primary CD4+ T cells at (7A) 0-11.5 hours, and (7B) 15-24 hours after thawing and culturing in RPMI medium with 10% FBS.

FIG. 8 illustrates binding of 10 nM A1 (red) and random aptamer (RAN, gray) to CD8+ T cells, monocytes, NK cells and B cells in three human apheresis products from three healthy donors. CD8+ T cells were gated on CD8 hi cells. Monocytes were gated on CD14+ cells. NK cells were gated on CD56+ cells. B cells were gated on CD19+ cells. PE: phycoerythrin.

FIGS. 9A, 9B illustrate (9A) Binding of 10 nM A1 and random aptamer (RAN) to J.RT3-T.35, Jurkat and H9 human T cell lines; and (9B) Binding curve of A1 to J.RT3-T.35 cells.

FIG. 10 illustrates a colloidal blue-stained SDS-PAGE gel of J.RT3-T3.5 membrane proteins pulled down by A1 or by biotin-saturated magnetic beads (control).

FIGS. 11A-11D illustrate characterization of A1 binding to CD62L. (11A) Overlaid flow cytometry plots of unstained, Cy5-labeled A1 stained, Alexa Fluor 488-labeled CD62L antibody (clone DREG-56) stained, and both Cy5-labeled A1 and Alexa Fluor 488-labeled CD62L antibody (clone DREG-56) stained J.RT3-T3.5 cells. (11B) Competitive binding of varying concentrations of A1 with 50 nM CD62L antibody (clone DREG-56) to J.RT3-T3.5 cells. (11C) Binding of A1 and a CD62L antibody (clone DREG-56) to J.RT3-T3.5 cells with CD62L expression knocked down by siRNA. (11D) Binding kinetics of 50 nM A1 immobilized on Streptavidin tips to varying concentrations of recombinant human CD62L by bio-layer interferometry (BLI).

FIGS. 12A, 12B illustrate secondary structures of aptamers predicted by (12A) NUPACK 4 and (12B) NUPACK 3.

FIGS. 13A-C illustrate (13A) aptamer truncation from CD62LApt.88 to CD62LApt.52 based on NUPACK-predicted secondary structures; (13B) binding kinetics of 50 nM CD62LApt.52 immobilized on Streptavidin tips to varying concentrations of recombinant human CD62L by bio-layer interferometry (BLI); and (13C) flow cytometry binding curves of CD62LApt.88 and CD62LApt.52 to J.RT3-T3.5 cells.

FIG. 14 illustrates binding curves of CD62LApt.88, CD62LApt.71, CD62LApt.52, and CD62LApt.42 to J.RT3-T3.5 cells.

FIGS. 15A, 15B illustrate binding kinetics of 50 nM CD62LApt.52 immobilized on Streptavidin tips to varying concentrations of (15A) recombinant human CD62E and (15B) recombinant human CD62P by bio-layer interferometry (BLI).

FIGS. 16A-16C illustrate (16A) binding kinetics of 50 nM LD174t1, LD201t1, and sgc3b (left to right) immobilized on Streptavidin tips to varying concentrations of recombinant human CD62L by bio-layer interferometry (BLI); (16B) binding curves of LD174t1, LD201t1, and sgc3b to J.RT3-T3.5 cells; and (16C) Competitive binding of varying concentrations of LD174t1, LD201t1, and sgc3b with 10 nM CD62LApt.52 to J.RT3-T3.5 cells.

FIG. 17A, 17B illustrate (17A) NUPACK-predicted disruption of CD62LApt.52's secondary structures via toehold-mediated strand displacement after reversal agent addition; and (17B) flow cytometry binding of % CD62LApt.52 binding to J.RT3-T3.5 cells after incubation with varying molar excess of reversal agent for 10 min at RT.

FIGS. 18A-18D illustrate (18A) flow cytometry plots of CFSE+J.RT3-T3.5 cell selection from a mixture of 40% J.RT3-T3.5 cells and 60% K562 cells using CD62LApt.52; (18B) NUPACK-predicted secondary structure of CD62LApt.5.2s20; (18C) flow cytometry binding curve of CD62LApt.52.s20 to J.RT3-T3.5 cells; and (18D) flow cytometry plots of CFSE+J.RT3-T3.5 cell selection from a mixture of 40% J.RT3-T3.5 cells and 60% K562 cells using CD62LApt.52.s20.

FIGS. 19A, 19B illustrate (19A) flow cytometry analysis of CFSE+J.RT3-T3.5 cell selection from a mixture of 40% J.RT3-T3.5 cells and 60% K562 cells using varying concentrations of CD62LApt.52.s20; and (19B) flow cytometry analysis of the distribution of target J.RT3-T3.5 in each selection fraction using 5× molar excess reversal agent, 50× molar excess reversal agent and 100× molar excess reversal agent+2-step elution.

FIG. 20 illustrates example CD8 staining of cell populations during a double-aptamer isolation procedure.

FIGS. 21A, 21B illustrate representative flow cytometry analysis of CD62L expression (x-axis) and CD8 expression (y-axis) in each selection fraction from (21A) aptamer-based serial positive selection strategy and (21B) antibody-based negative selection-then-positive selection strategy.

FIGS. 22A-22E illustrate flow cytometry analysis of (22A) CD62L+CD8+ purity, (22B) CD62L+CD8+CD3+ purity, (22C) overall CD62L+CD8+ cell yield, (22D) CD8+ cell yield, and (22E) CD62L+ cell yield. The individual data points represent different PBMC donors from separate isolation experiments. Horizontal lines and error bars represent the mean s.d.; n=3; P>0.05, *P<0.05, **P<0.01, ***P<0.001 (paired one-way ANOVA with Tukey's test).

FIG. 23 illustrates flow cytometry analysis of the cell phenotypes in the reversal agent elution fraction after CD8 aptamer selection and CD62L aptamer selection. Naïve CD8 T cells are CD8+CD3+CD45RA+CD45RO−/CCR7+ or CD62L+, transition cells are CD8+CD3+CD45RA+CD45RO+, central memory CD8 T cells are CD8+CD3+CD45RA−CD45RO+/CCR7+ or CD62L+, effector memory CD8 T cells are CD8+CD3+CD45RA-CD45RO+CCR7−CD62L−, terminal effector memory CD8 T cells are CD8+CD3+CD45RA+CD45RO−CCR7−CD62L−. Data are mean±s.d., n=3, *P<0.05, ***P<0.001 and ****P<0.0001 (paired two-way ANOVA).

FIG. 24. Sequences supporting the disclosure, including 52-nucleotide L-selectin aptamer (SEQ ID NO: 1), 61-nucleotide L-selectin aptamer (SEQ ID NO: 2), 72-nucleotide L-selectin aptamer (SEQ ID NO: 3), 88-nucleotide L-selectin aptamer (SEQ ID NO: 4), 72-nucleotide L-selectin aptamer (SEQ ID NO: 5), 42-nucleotide L-selectin aptamer (SEQ ID NO: 6), Reversal construct (SEQ ID NO: 7), random aptamer (SEQ ID NO: 8), forward primer (SEQ ID NO: 9), Reverse primer_Round 2 (SEQ ID NO: 10), Reverse primer_Round 3 (SEQ ID NO: 11), Reverse primer_Round 4 (SEQ ID NO: 12), Reverse primer_Round 5 (SEQ ID NO: 13), Reverse primer_Round 6 (SEQ ID NO: 14), Reverse primer_Round 7 (SEQ ID NO: 15), Reverse primer_Round 8 (SEQ ID NO: 16), hs.Ri.SELL.13.1-SEQ1 (SEQ ID NO: 17), hs.Ri.SELL.13.1-SEQ2 (SEQ ID NO: 18), hs.Ri.SELL.13.2-SEQ1 (SEQ ID NO: 19), hs.Ri.SELL.13.2-SEQ2 (SEQ ID NO: 20), and random region sequences (SEQ ID NO: 21 to SEQ ID NO:51).

### DETAILED DESCRIPTION

Previously, the dominant method for cell isolation in CAR T cell therapy manufacturing was antibody-based, positive selection using magnetic beads, a process known as magnetic activated cell sorting (MACS) (Lu, T. L., et al., Hum. Gene Ther. Methods, 2016. 27(6): p. 209-218; Marques, G. S., et al., Biol. Proced. Online, 2018. 20(1): p. 1-9; Spohn, G., et al., Cytotherapy, 2015. 17(10): p. 1465-1471). With an exception of the Streptamer technology which allows for displacement of antibody-functionalized magnetic beads, (Sabatino, M., et al., Blood, 2016. 128(4): p. 519-528; Stemberger, C., et al., PloS one, 2012. 7(4): p. e35798), the associated magnetic beads preclude subsequent MACS separation with the cells. As a result, selection of cell subsets includes a combination of positive and negative selection strategies. The necessity for multiple antibody-based isolation procedures to obtain naïve and memory T cells would add significant cost to the manufacturing process.

An aptamer-based method for isolating CD8 T cells was previously reported (Kacherovsky, N., et al., Nat. Biomed. Eng., 2019. 3(10): p. 783-795). Aptamers, which include nucleic acid sequences that fold into secondary structures, can bind to target proteins with affinities comparable to antibodies but are chemically produced at a fraction of the cost of antibodies (Bunka, D. H. J. and P. G. Stockley, Nat. Rev. Microbiol., 2006. 4: p. 588; I Hernandez, L., et al., Curr. Top. Med. Chem., 2015. 15(12): p. 1066-1081; Zhou, J. and J. Rossi, Nat. Rev. Drug Discovery, 2016. 16: p. 181). Previously, a high affinity CD8-binding aptamer (CD8Apt) was identified and designed a modified aptamer containing a toehold sequence complementary to a reversal agent (RA) capable of initiating strand displacement. This system was successfully applied to isolate high purity, traceless CD8 T cells.

Various implementations described herein relate to constructs, and other types of structures, including oligonucleotides that specifically bind to L-selectin. In some implementations, the constructs include aptamers with particular binding domains that bind to L-selectin. According to some examples, the constructs bind to soluble and/or cell-expressed L-selectin, such as L-selectin expressed by CD62L+ T cells.

For instance, a construct includes an example oligonucleotide that specifically binds L-selectin and includes a 52-nucleotide sequence that is provided by SEQ ID NO: 1 (TAGTCAAGGCGATAGACGAAAAACATTGTTTTCCTGGAAGGCCCAGAATCTG). Other example sequences are also discussed herein. A 61-nucleotide sequence of an example oligonucleotide that specifically binds L-selectin is provided by SEQ ID NO: 2 (AGACCCGACGCAGCATAGTCAAGGCGATAGACGAAAAACATTGTTTTCCTGGAAGGCCCAGAATCTGCG). A 71-nucleotide sequence of an example oligonucleotide that specifically binds L-selectin is provided by SEQ ID NO: 3 (CCAGAGTGACGCAGCATAGTCAAGGCGATAGACGAAAAACATTGTTTTCCTGGAAGGCCCAGAATCTGTGG).

An 88-nucleotide sequence of an example oligonucleotide that specifically binds L-selectin is provided by SEQ ID NO: 4 (ATCCAGAGTGACGCAGCATAGTCAAGGCGATAGACGAAAAACATTGTTTTCCTGGAAGGCCCAGAATCTGTGG ACACGGTGGCTTAGT). A 72-nucleotide sequence of an example oligonucleotide that specifically binds L-selectin is provided by SEQ ID NO: 5 (TTTTTTTTTTTTTTTTAATTTAGTCAAGGCGATAGACGAAAAACATTGTTTTCCTGGAAGGCCCAGAATC TG). An additional 42-nucleotide sequence will also be described herein and is provided by SEQ ID NO: 6 (CTGGGCGATAGACGAAAAACATTGTTTTCCTGGAAGGCCCAG). In various implementations of the present disclosure, the constructs include oligonucleotide sequences having a length of greater than 42 nucleotides, such as greater than 45 nucleotides or greater than 50 nucleotides. In some cases, the oligonucleotide sequences have a length of less than 100 nucleotides, such as less than 90 nucleotides.

Example constructs can additionally include other components. For instance, a construct in accordance with implementations of the present disclosure can include one or more of the oligonucleotide sequences described above bound to a magnetic bead, microbubble, or other substrate. In some cases, the construct specifically binds to L-selectin expressed by CD62L+ T cells and can be used to isolate CD62L+ T cells from other types of cells. In some implementations, the construct includes one or more of the oligonucleotide sequences linked to another chemical, such as polyethylene glycol (PEG) or to a small molecule such as a lipid, in order to improve the circulation time and/or bioavailability of the construct in vivo.

In some implementations, the construct includes one or more of the oligonucleotide sequences linked to at least one tag (e.g., chromophore tags, quantum dots, fluorescent molecules, etc.). According to various cases, the oligonucleotide sequence(s) change shape when binding to L-selectin. Due to this conformal change, the tag(s) may emit a detection signal (e.g., light) when the oligonucleotide sequence(s) bind to L-selectin. For example, the construct may be utilized as a fluorescence resonance energy transfer (FRET) probe. In some cases, the construct is utilized in a sandwich assay. For instance, the construct binds L-selectin while the construct is immobilized on paper, glass, a polymer surface, a resin, or some other substrate. Another detection construct, such as a detection antibody or aptamer, may also bind to the L-selectin or another structure bound to the L-selectin (e.g., a cell). When the detection construct encounters a tag, the detection construct may cause the tag to emit a detection signal. Therefore, by detecting any of the detection signals described herein, the construct may be used to identify when the construct binds to L-selectin and/or how many constructs bind to L-selectin in a given sample. Accordingly, various implementations of the present disclosure can be utilized to detect L-selectin.

In various cases, the construct can be utilized therapeutically. In various cases, the construct binds to L-selectin expressed by cells. In some cases, the bound construct deactivates the L-selectin. Deactivation of L-selectin, in various cases, can suppress one or more inflammatory pathways in a subject. In some examples, the construct is linked to a binding domain for an antigen. The antigen may be associated with a pathology of the subject. The construct, in various cases, binds cells expressing L-selectin and brings the cells expressing L-selectin in proximity to cells expressing the antigen. In some examples, the cells expressing L-selectin induce cell death in the cells expressing the antigen. Thus, according to some implementations, the construct can be utilized to treat pathologies and/or other conditions associated with L-selectin functionality.

Implementations of the present disclosure have various advantages over existing technologies. Various oligonucleotides described herein can be manufactured less expensively than antibodies that specifically bind L-selectin. Moreover, various oligonucleotides described herein reversibly bind L-selectin. For example, an example oligonucleotide bound to L-selectin will be released from L-selectin in the presence of a reversal agent (also referred to as an “antidote” or “reversal construct”). An example reversal agent includes an oligonucleotide sequence provided by SEQ ID NO: 7 (CAGATTCTGGGCCTTCCAGGAAAACAAT). Because implementations of the present disclosure can be utilized to reversibly bind to L-selectin (or L-selectin expressing cells), they can be utilized to reversibly and tracelessly isolate L-selectin expressing cells, detect L-selectin, and treat L-selectin-related conditions.

Various implementations of the present disclosure will now be described with reference to the accompany figures.

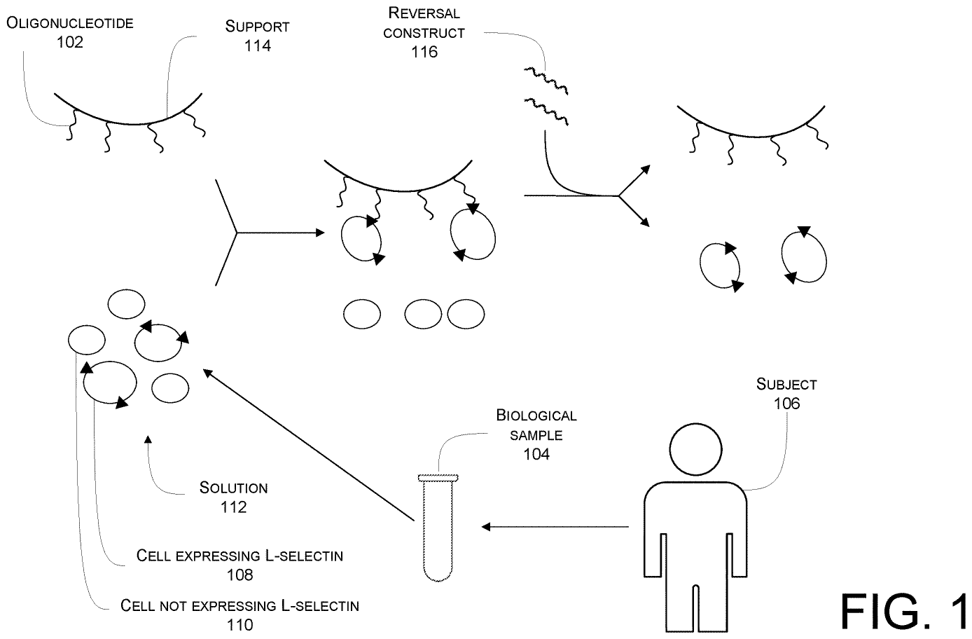

FIG. 1 illustrates an example environment for cell selection using an oligonucleotide 102 that specifically binds L-selectin. “Cell selection” or “cell sorting”, in various implementations, can refer to a process of isolating one or more specific cells from a population of cells. In some examples, the one or more specific cells include cells of a particular cell type. In some examples, the one or more specific cells include cells of more than one particular cell type. A cell type, in some implementations, includes cells that express a particular molecular marker (e.g., an antigen, a protein, or a genetic marker). In various examples, a biological sample 104 may be taken from a subject 106. In various examples, the subject 106 may be a human or other animal. In some examples, the subject 106 suffers from a pathology associated with a cell expressing L-selectin. In some cases, the subject 106 has at least one of sepsis, organ rejection, cancer, infection, inflammation, or an autoimmune condition. In some examples, the autoimmune disease is a T-cell-mediated autoimmune disease. Examples of T-cell mediated autoimmune conditions include diabetes, multiple sclerosis, and rheumatoid arthritis. In some examples, the subject 106 is a donor.

L-selectin, also referred to as “CD62L,” is a transmembrane glycoprotein that is involved in cell adhesion and migration of immune cells. L-selectin expressed on the cell surface can be referred to as cell-expressed L-selectin. L-selectin is expressed on the cell surface of most circulating leukocytes. “Expression,” as used herein, can refer to the presence of a particular protein in a cell. A protein can be expressed within a cell or on the surface of the cell. L-selectin that is not expressed on a cell surface is referred to as soluble L-selectin. In some examples, L-selectin is shed from the cell surface to form soluble L-selectin. Both cell-expressed L-selectin and soluble L-selectin can be associated with pathologies described herein.

In various examples, the biological sample 104 includes a mixture of cells. For instance, the biological sample 104 may include cells of more than one cell type. The biological sample 104 may include immune cells (e.g., lymphocytes, granulocytes, monocytes, etc.), red blood cells, epithelial cells, fat cells, muscle cells, and/or stem cells. The biological sample 104 may include a cell expressing L-selectin 108. In some examples, L-selectin is expressed on the surface of the cell expressing L-selectin 108. In various instances, the biological sample 104 includes a cell not expressing L-selectin 110. The biological sample 104 may be a blood sample, a tissue sample, or another sample that includes cells of the subject 106. In some examples, the biological sample 104 includes T cells. The T cells may include at least one of naïve T cells (T_{N}), stem cell memory T cells (T_{SCM}), and central memory T cells (T_{CM}). The T cells may include CD62L+ T cells. In various implementations, the cell expressing L-selectin 108 is a T_{N}, a T_{SCM}, a T_{CM}, or a CD62L+ T cell.

In some cases, it may be beneficial to isolate the cell expressing L-selectin 108. For example, the subject 106 may suffer from a pathology, and a condition of the subject 106 may be improved by administering modified cells that express L-selectin. For example, the subject may have cancer and may benefit from treatment including T cells that have been modified to express a chimeric antigen receptor (CAR) that targets cancer cells. In some cases, the efficacy of CAR T cell treatment is improved by exclusively, or primarily, using T cells that express L-selectin.

It is possible to isolate an individual cell type using antibodies that specifically bind to a marker expressed by the individual cell type. For example, anti-CD4 antibodies can be used to capture and isolate T cells that express the CD4 glycoprotein. However, antibody-based cell selection techniques have limitations, including selection difficulties, selectivity problems (e.g., cross-reactivity, off-target binding, and batch-to-batch variation), preparation difficulties, high costs of production, stability issues, and lengthy production times. Furthermore, due to the selectivity problems associated with antibodies, cell selection may involve more than one round of selection, including positive and/or negative selection. In some cases, additional reagents and processing time may be utilized to release the cells bound to the antibodies. In certain cases, such as when developing a treatment for a patient who is severely ill, minimizing processing time may be beneficial to improving a patient's chance of survival.

These issues can be addressed by using oligonucleotides for cell selection. In various implementations, cell selection using oligonucleotides offers several benefits over antibody-based techniques, including simple and inexpensive production, strong specificity and discrimination of molecular differences, longer shelf life, batch-to-batch consistency, and capacity for chemical modification.

In various implementations of the present disclosure, the oligonucleotide 102 is used for selection of cells expressing L-selectin. “Oligonucleotide” or “aptamer,” as used herein refers to a single-stranded nucleic acid. The oligonucleotide 102, in various examples, specifically binds L-selectin. In some examples, the oligonucleotide 102 includes a sequence having at least 70% sequence identify to SEQ ID NO: 1, SEQ ID NO: 2, SEQ ID NO: 3, SEQ ID NO: 4, SEQ ID NO: 5, or SEQ ID NO: 6. In some implementations, a length of the oligonucleotide 102 is in a range of 30 to 200 nucleotides or in a range of 40 to 90 nucleotides. In some examples, the oligonucleotide 102 includes a sequence having at least 75%, 80%, 85%, 90%, 95%, 97%, or 99% sequence identify to SEQ ID NO: 1, SEQ ID NO: 2, SEQ ID NO: 3, SEQ ID NO: 4, SEQ ID NO: 5, or SEQ ID NO: 6. In some implementations, the oligonucleotide includes the sequence as set forth in SEQ ID NO: 1, SEQ ID NO: 2, SEQ ID NO: 3, SEQ ID NO: 4, SEQ ID NO: 5, or SEQ ID NO: 6.

In some cases, the oligonucleotide 102 is conjugated to a support 114, a tag, or a linker. Examples of the support 114 include paper, glass, a polymer, a resin, or a particle. Examples of tag include a fluorophore, biotin, a dye, a chromophore tag, a quantum dot, or a radionuclide. In some examples, the oligonucleotide 102 is conjugated to a linker conjugated to a second oligonucleotide.

In various implementations, the biological sample 104 is exposed to the oligonucleotide 102 to isolate the cell expressing L-selectin 108. Based on exposing the biological sample 104 to the oligonucleotide 102, the oligonucleotide 102 may bind the cell expressing L-selectin 108. In various implementations, a binding affinity of the oligonucleotide 102 to the cell expressing L-selectin 108 is in a range of 0.005 to 100 nanomolar (nM). In some examples, the binding affinity of the oligonucleotide 102 to the cell expressing L-selectin 108 is in a range of 0.01 to 5 nM. In some implementations, a binding affinity of the oligonucleotide 102 to L-selectin is in a range of 1 to 1000 nM. In various examples, the binding affinity of the oligonucleotide 102 to L-selectin is in a range of 4 to 100 nM.

In some examples, the biological sample 104 is incubated with the oligonucleotide 102. For example, a solution 112 including the mixture of cells may be produced from the biological sample 104. In some instances, the solution 112 may be produced by generating a cell suspension from the biological sample 104, filtering the biological sample 104, diluting the biological sample 104, washing the biological sample 104 with a buffer and/or performing lysis of one or more cells that do not express L-selectin (e.g., red blood cells, the cell not expressing L-selectin 110, etc.). In various cases, filtering can be done manually or using a machine. Filtering may include gravity filtration, centrifugation, vacuum filtration, pressure filtration, microfiltration, or another suitable filtration method. In some examples, the oligonucleotide 102 is conjugated to a magnetic bead (e.g., the support 114) and incubated with the solution 112 for a time. The time, in some cases, is determined based on binding kinetics of the oligonucleotide 102 and L-selectin. During the time, the oligonucleotide 102 may bind the cell expressing L-selectin 108.

The cell expressing L-selectin 108 is isolated, in some examples, by applying a magnetic field to the solution 112. The magnetic field may be produced by a ferromagnetic material and/or an electromagnet. For example, the solution 112 may be in a container, and a ferromagnetic material may be placed on the outer surface of the container. In some examples, the container includes, or is disposed within a field generated by, an electromagnet. An electric current can be applied to the electromagnet to produce a magnetic field. The magnetic field causes the magnetic beads to align with the magnetic field and form a cluster within the solution 112. In some examples, the magnetic beads aggregate toward a pole of a magnetic material or the source of the magnetic field. In some examples, the aggregated magnetic beads can be removed to isolate the cells expressing L-selectin 108. In some examples, the aggregated magnetic beads can be immobilized by the magnet and the cells not expressing L-selectin 110 may be removed by decanting, draining, aspirating, washing, or another suitable method.

In some examples, the oligonucleotide 102 is conjugated to a microbubble (e.g., the support 114) including a shell encapsulating a gas. In some cases, the shell can include lipids, proteins, polymers (e.g., poly(lactic co-glycolic acid) (PLGA)), lipopolymers, or phospholipids. The gas, in various examples, may include air, nitrogen, or a perfluorocarbon (e.g., perfluorobutane or perfluoropropane). A size of the microbubble, in some examples, is in a range of 1 to 10 μm. The oligonucleotide 102 may be mixed into the solution 112. Based on mixing the oligonucleotide 102 into the solution 112, the oligonucleotide 102 may bind the cell expressing L-selectin 108. Due to the buoyancy of the microbubble, the cell expressing L-selectin 108 may float to the surface of the solution 112. In some cases, the cell expressing L-selectin 108 is isolated by aspiration. Aspiration, as used herein, can refer to the process of collecting a component of a solution, wherein the component is generally located at the surface of the solution. In various examples, the aspiration can be performed manually (e.g., using a pipette or syringe), by using a vacuum aspiration system, or by using another appropriate system.

In various examples, the oligonucleotide is immobilized to paper, glass, or a polymer (e.g., the support 114).

The solution 112 may be washed over the support 114 or incubated with the support 114. In various instances, the cell expressing L-selectin 108 binds to the oligonucleotide 102. The cell expressing L-selectin 108 is isolated, for instance, by removing the support 114 from the solution 112 or by applying a buffer to the support 114 to remove one or more cells not expressing L-selectin 110.

In some examples, the oligonucleotide 102 is conjugated to one or more fluorophores. In various cases, the oligonucleotide 102 is conjugated to two or more fluorophores. The solution 112 may be incubated with the oligonucleotide 102, and the oligonucleotide 102 may bind the cell expressing L-selectin 108. The binding of the oligonucleotide 102 to the cell expressing L-selectin 108, for instance, causes a conformational change of the oligonucleotide 102. Based on the conformational change, a distance between the two fluorophores may decrease, causing a transfer of energy between the two fluorophores. Based on the transfer of energy, the fluorescence emission spectra of the oligonucleotide 102 may change. The cell expressing L-selectin 108 can be isolated by detecting the fluorescence emission spectra. In some examples, the cell expressing L-selectin 108 is isolated by performing fluorescence activated cell sorting (FACS).

In various implementations, based on the cell expressing L-selectin 108 being bound to the oligonucleotide 102, the cell expressing L-selectin 108 is dissociated from the oligonucleotide 102. The cell expressing L-selectin 108 can be dissociated from the oligonucleotide 102, for example, by introducing a reversal construct 116 to the solution 112, changing a pH of the solution 112 (e.g., by adding an acid or a base to the solution 112), changing a concentration of salt in the solution 112, changing a temperature of the solution 112, applying a mechanical force to the solution 112, or introducing a nuclease enzyme to the solution 112. In various implementations, any change in the pH of the solution 112 is sufficient to dissociate the cell expressing L-selectin 108 from the oligonucleotide 102. In some examples, changing the solution 112 to be more basic may dissociate the cell expressing L-selectin 108 from the oligonucleotide 102. In some examples, changing the solution 112 to be more acidic may dissociate the cell expressing L-selectin 108 from the oligonucleotide 102. In various examples, the pH of the solution 112 may be increased by 0.1, 0.2, 0.5, 1, 2, 3, 4, 5, 6, 7, 8, 9, or 10 to dissociate the cell expressing L-selectin 108 from the oligonucleotide 102. In various examples, the pH of the solution 112 may be decreased by 0.1, 0.2, 0.5, 1, 2, 3, 4, 5, 6, 7, 8, 9, or 10 to dissociate the cell expressing L-selectin 108 from the oligonucleotide 102. In various implementations, the mechanical force can be applied manually or using a machine (e.g., an orbital shaker, a platform shaker, a shaking incubator, or another suitable machine).

The reversal construct 116, in various implementations, includes a second oligonucleotide. In some examples, the second oligonucleotide includes a sequence having at least 70% sequence identity to SEQ ID NO: 7.

In some examples, the second oligonucleotide includes a sequence having at least includes a sequence having at least 75%, 80%, 85%, 90%, 95%, 97%, or 99% sequence identify to SEQ ID NO: 7. In some examples, the second oligonucleotide includes the sequence as set forth in SEQ ID NO: 7.

In various implementations, the cell expressing L-selectin 108 is modified to treat the subject 106. For example, the subject 106 may have cancer, and cancer cells expressing an antigen may be present in the subject 106. The cell expressing L-selectin 108 is, in some examples, transduced with a vector encoding a chimeric antigen receptor (CAR). In some cases, the CAR is designed to specifically bind the antigen expressed by the cancer cells of the subject 106. In various examples, the cell expressing L-selectin 108 transduced with the CAR is a CAR T cell.

The CAR T cells may be infused into the subject 106, and the CAR may bind to one or more cancer cells in the subject. Based on binding the one or more cancer cells, the CAR may activate the CAR T cell to cause cell death of the one or more cancer cells.

In particular implementations, CAR T cells can be formulated into a carrier in a therapeutically-effective amount. As described herein, exemplary carriers include saline, buffered saline, physiological saline, water, Hanks' solution, Ringer's solution, Normosol-R (Abbott Labs), PLASMA-LYTE A® (Baxter Laboratories, Inc., Morton Grove, IL), and combinations thereof.

Cells (e.g., the cell expressing L-selectin 108) and or other components described herein may be administered in a formulation that includes one or more carriers, stabilizers, anesthetics, preservatives, or any combinations thereof.

In particular implementations, carriers can be supplemented with human serum albumin (HSA) or other human serum components or fetal bovine serum. In particular implementations, a carrier for infusion includes buffered saline with 5% HSA or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where beneficial, formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

In some examples, it may be beneficial to isolate cells expressing more than one antigen. For instance, it may be beneficial to isolate cells expressing a first antigen (e.g., L-selectin) and a second antigen (CD8). For example, it may be beneficial to isolate cells expressing L-selectin and CD8 (e.g., CD8+CD62L+ T cells). In some cases, selection of CD8+CD62L+ T cells may improve the purity of the CD8+ cell population.

In various implementations, a second solution including the cell expressing L-selectin 108 is exposed to a third oligonucleotide that specifically binds the second ligand. The second ligand, in various examples, is associated with the pathology. For example, the second ligand may be CD8. In some examples, the second ligand may be CD4. In some examples, the third oligonucleotide is conjugated to a disclosed support, a disclosed tag, or a disclosed linker. In some cases, a cell expressing L-selectin and the second ligand binds to the third oligonucleotide. The bound cell expressing L-selectin and the second ligand can be isolated as described herein.

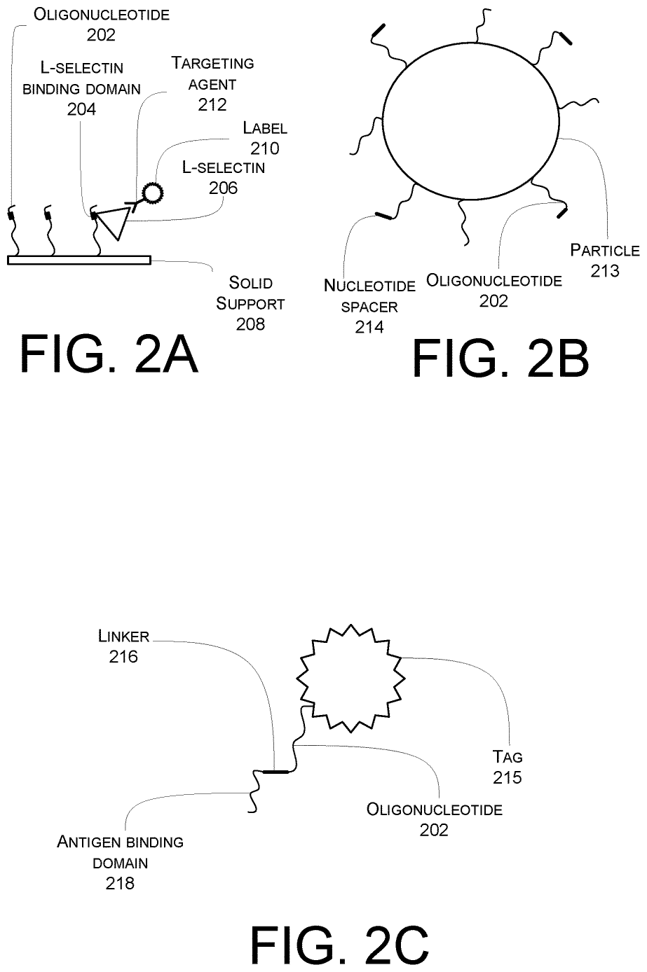

FIGS. 2A-2C illustrate an example oligonucleotide 202 for diagnosis or treatment of a pathology associated with L-selectin. In various implementations, the oligonucleotide 202 specifically binds L-selectin. In some cases, the oligonucleotide 202 includes a sequence having at least 70%, 75%, 80%, 85%, 90%, 95%, 97%, or 99% sequence identify to SEQ ID NO: 1, SEQ ID NO: 2, SEQ ID NO: 3, SEQ ID NO: 4, SEQ ID NO: 5, or SEQ ID NO: 6. In various implementations, the oligonucleotide 202 is conjugated to a construct. In various examples, the construct is a solid support 208 (illustrated in FIG. 2A), a particle 213 (illustrated in FIG. 2B), a tag 215 (illustrated in FIG. 2C), a linker 216 (illustrated in FIG. 2C), or a drug.

In some examples, the oligonucleotide 202 is administered to a subject or applied to a biological sample collected from the subject. In various cases, the subject suffers from a pathology associated with L-selectin. In some examples, L-selectin or cells that express L-selectin are present in the body of the subject. In some cases, the subject has at least one of sepsis, organ rejection, cancer, infection, inflammation, or an autoimmune condition. In some examples, the autoimmune disease is a T-cell-mediated autoimmune disease. Examples of T-cell mediated autoimmune conditions include diabetes, multiple sclerosis, and rheumatoid arthritis.

In various implementations, the oligonucleotide 202 includes an L-selectin binding domain 204 that specifically binds L-selectin 206. In some examples, the oligonucleotide 202 may include one or more binding domains that do not bind L-selectin. In some cases, L-selectin 206 may bind to one or more binding domains that are not the L-selectin binding domain 204.

FIG. 2A illustrates an example oligonucleotide 202 conjugated to a solid support 208. In various implementations, the oligonucleotide 202 is conjugated to the solid support 208 for detection of L-selectin 206 in the biological sample. The L-selectin 206 may be soluble L-selectin or cell-expressed L-selectin. The oligonucleotide 202 may be exposed to the biological sample. The solid support 208 may be paper, glass, a polymer, or a resin.

In various examples, the paper may be made of cellulose, nitrocellulose, a polymer (e.g., high-density polyethylene (HDPE), glass fiber, other plant-based materials, or other appropriate materials. In some cases, the paper may be treated. For example, the paper may be treated to improve adsorption of the oligonucleotide 202 onto the paper. In some examples, the paper may be treated to reduce non-specific binding, to improve durability or stability, or for another appropriate purpose. In various examples, the polymer may include polyethylene glycol (PEG), dextran, poly(lactic-co-glycolic acid) (PLGA), polylactic acid (PLA), polyglycolic acid (PGA), polyvinyl Alcohol (PVA), polyvinylpyrrolidone (PVP), poly(dl-lactide) (PDLLA), or poly(N-isopropylacrylamide) (PNIPAM), or another suitable polymer. In various examples, the resin may be an agarose resin, a sepharose resin, a polystyrene resin, a silica resin, a nylon resin, or another suitable resin. In some cases, the resin may be coated or modified to improve function. For example, the resin may be coated with streptavidin, biotin, Protein A, Protein G to improve conjugation of the oligonucleotide 202. In some examples, the resin may be modified to include epoxy groups, N-hydroxysuccinimide (NHS) groups, aldehyde groups, or amino groups to improve conjugation of the oligonucleotide 202.

In some implementations, a solution including cells from the biological sample is generated. The solution is generated, in various instances, by generating a cell suspension from the biological sample, filtering the biological sample, diluting the biological sample, washing the biological sample with a buffer, and/or performing lysis of one or more cells that do not express L-selectin (e.g., red blood cells). The oligonucleotide 202 may be added to the solution.

In some examples, the oligonucleotide 202 is conjugated to a paper. The solution may be applied to the paper (e.g., the solid support 208), and L-selectin 206 in the solution may bind the oligonucleotide 202. Unbound cells and/or proteins are, in some cases, removed from the paper using a buffer. In various cases, a detection agent comprising a label 210 (e.g., a fluorophore, biotin, a dye, or a radionuclide, a nanoparticle, a quantum dot, a chromophore tag, an enzyme, etc.) conjugated to a targeting agent 212 (e.g., an antibody, an oligonucleotide, etc.) is applied to the paper. The targeting agent 212 may bind the L-selectin 206, and the label 210 can be detected to identify L-selectin 206 on the paper. In some cases, the label 210 is an enzyme, such as horseradish peroxidase (HRP), alkaline phosphatase (AP), glucose oxidase (Gox), or another suitable enzyme. In various implementations, a substrate may be applied to the paper to activate the enzyme. Based on activating the enzyme, the enzyme may catalyze a reaction with the substrate, thereby generating a signal (e.g., a colorimetric signal, a light signal, or another signal). In some examples, the label 210 or the signal can be detected using a fluorometer, a plate reader, or another imaging system. In various cases, the label 210 or the signal is detected by visual inspection or using spectroscopy, surface-enhanced Raman scattering, surface plasmon resonance, or another technique.

In some implementations, the oligonucleotide 202 is conjugated to the solid support 208 for treatment of the pathology. For example, the pathology may be associated with activated T cells or cells that overexpress L-selectin.

In some examples, the pathology is an autoimmune disease, organ rejection, or another pathology associated with activated T cells. In various implementations, the subject may benefit from removing the activated T cells from the body of the subject. For example, the biological sample may be exposed to the oligonucleotide 202. Based on exposing the biological sample to the oligonucleotide 202, a cell expressing L-selectin may bind to the oligonucleotide 202. The cell expressing L-selectin can be removed from the biological sample by removing the solid support 208 or by aspirating the unbound cells. The unbound cells, in various examples, do not express L-selectin. The unbound cells can be administered to the subject for treatment of the pathology.

FIG. 2B illustrates the example oligonucleotide 202 conjugated to a particle 213. In various example, the oligonucleotide 202 can be used for diagnosis or treatment of the pathology. In various implementations, the particle 213 may be a nanoparticle, a microsphere, a bead, or a microbubble. In some examples, the oligonucleotide 202 includes a nucleotide spacer 214. A length of the nucleotide spacer, in various cases, is in a range of 1 to 50 nucleotides. In some examples, the nucleotide spacer 214 is a polythymine spacer. The particle 213 may be exposed to the biological sample or a solution of cells generated from the biological sample. Based on exposing the particle 213 to the biological sample, a cell expressing L-selectin may bind the oligonucleotide 202. The cell expressing L-selectin may be isolated by manipulating the particle 213. In various examples, manipulating includes causing a movement of the particle 213, immobilization of the particle 213, a structural change of the particle 213, binding of the particle 213 to a ligand or to a second construct, or another suitable technique.

For example, the oligonucleotide 202 may be conjugated to a magnetic bead (e.g., the particle 213), and based on adding the oligonucleotide 202 to the solution of cells, a cell expressing L-selectin may bind the oligonucleotide 202. The cell expressing L-selectin may be isolated by applying a magnetic field to the solution of cells. In some examples, the cells that do not express L-selectin may be removed from the solution of cells by decanting, draining, aspirating, washing, or another appropriate method. In some examples, the oligonucleotide 202 can be used to remove activated T cells from the body of the subject as described in FIG. 2A.

FIG. 2C illustrates the example oligonucleotide 202 conjugated to a tag 215. In various examples, the tag 215 is a fluorophore, biotin, a dye, or a radionuclide, a nanoparticle, a quantum dot, a chromophore tag, or another detectable tag. In various examples, cells from the biological sample are immobilized on a surface. The oligonucleotide 202 may be conjugated to a nanoparticle (e.g., the tag 215, a metal nanoparticle, a metal oxide nanoparticle, a polymeric nanoparticle, a liposome, a micelle, a quantum dot, a carbon-based nanoparticle, or another nanoparticle) and may be applied to the immobilized cells. The oligonucleotide 202 may bind an immobilized cell that expresses L-selectin. In various cases, unbound oligonucleotides may be removed from the surface, such as by washing. The cells expressing L-selectin are detected by visual inspection or using spectroscopy, surface-enhanced Raman scattering, surface plasmon resonance, or another appropriate technique. In some instances, the oligonucleotide 202 is conjugated to biotin, and the cells expressing L-selectin are detected using a streptavidin-conjugated label. In some examples, the oligonucleotide 202 is conjugated to a fluorophore, and the cells expressing L-selectin are detected using a microscope, a fluorometer, a plate reader, or another imaging system.

In some implementations, the oligonucleotide 202 is conjugated to the tag 215 for detection of cells expressing L-selectin in the subject. The subject, in some examples, has an auto-immune disease. The tag 215 may be a radionuclide or a dye. In some examples, the radionuclide may be fluorine-18 (F-18), yttrium-90 (Y-90), carbon-11 (C-11), gallium-68 (Ga-68), technetium-99m (Tc-99m), iodine-123 (1-123), Iodine-131 (1-131), Indium-111 (In-111), xenon-133 (xe-133), thallium-201 (Tl-201), or another radionuclide. In some examples, the dye may be a gadolinium-based dye, an iodine-based dye, barium sulfate, fluorescein, indocyanine green, or another dye. In various implementations, the oligonucleotide 202 is administered to the subject. The oligonucleotide 202 may bind a cell expressing L-selectin in the subject. The cell expressing L-selectin may be detected by performing medical imaging, such computed tomography (CT) imaging, positron emission tomography (PET) imaging, magnetic resonance imaging (MRI), single photon emission computed tomography (SPECT), or other medical imaging techniques.

In some implementations, the oligonucleotide 202 is conjugated to a linker 216 or a drug for treatment of the pathology. For example, the subject may have cancer and may benefit from the destruction of the cancer cells in the subject. The oligonucleotide 202 may be conjugated to the linker 216. The linker 216, in some examples, is configured to connect the oligonucleotide 202 to a second oligonucleotide, an antigen binding domain, a drug, a protein, a lipid, a peptide, a tag, a particle, a solid support, an antibody, an antibody fragment, a small molecule, or another suitable construct. The linker 216, in various cases, is an alkyne linker, an amino linker, a dicarboxylic acid linker, a phosphodiester linker, a phosphorothioate linker, a thiol linker, a carboxyl linker, a peptide nucleic acid (PNA) linker, triethylene glycol (TEG) linker, a click chemistry-compatible linker, a polyethylene glycol (PEG) linker, or another suitable linker. In some examples, the linker 216 is conjugated to an antigen binding domain (ABD) 218. For instance, to the ABD 218 may be a cancer antigen binding domain. The oligonucleotide-linker-ABD complex may be administered to the subject. The oligonucleotide-linker-ABD complex may, in some cases, facilitate the movement of cells expressing L-selectin (e.g., activated CD62L+ T cells) towards cancer cells. In various examples, based on the administration of the oligonucleotide-linker-ABD complex, the cells expressing L-selectin may attack and kill the cancer cells.

In some examples, the oligonucleotide 202 is conjugated to a drug for the treatment of the pathology. The drug may be a pharmaceutical drug or a therapeutic agent that is administered to the subject for the purpose of diminishing or eliminating signs or symptoms of the pathology. For example, the subject may have an auto-immune disease and may benefit from the destruction of cells expressing L-selectin (e.g., activated CD62L+ T cells) in the subject. In some examples, the drug is a cytotoxic drug. Examples of cytotoxic drugs include actinomycin D, an alkylating agent, anthracycline, auristatin, calicheamicin, camptothecin, CC1065, colchicin, cyclophosphamide, cytarabine, cytochalasin B, daunorubicin, 1-dehydrotestosterone, dihydroxy anthracinedione, dolastatin, doxorubicin, duocarmycin, elinafide, emetine, ethidium bromide, etoposide, gramicidin D, glucocorticoids, lidocaine, maytansinoid, mithramycin, mitomycin, mitoxantrone, nemorubicin, PNU-159682, procaine, propranolol, puromycin, pyrrolobenzodiazepine, taxane, taxol, tenoposide, tetracaine, trichothecene, vinblastine, vinca alkaloid, or vincristine. A therapeutically effective amount of the oligonucleotide may be administered to the subject. Based on the administration of the oligonucleotide, the drug may provide a cytotoxic effect on the cells expressing L-selectin.

Treating subjects includes delivering therapeutically effective amounts. Therapeutically effective amounts include those that provide effective amounts, prophylactic treatments and/or therapeutic treatments.

An “effective amount” is the amount of a formulation used to result in a desired physiological change in the subject. For example, an effective amount can provide a change in a metric associated with a pathology associated with L-selectin (e.g., a severity or frequency of symptoms or a change in a marker associated with the pathology). Effective amounts are often administered for research purposes. Effective amounts disclosed herein can cause a statistically significant effect in an animal model or in vitro assay relevant to the assessment of a pathology's development or progression.

A “prophylactic treatment” includes a treatment administered to a subject (e.g., the subject 106) who does not display signs or symptoms of a pathology associated with L-selectin or displays only early signs or symptoms of a pathology associated with L-selectin such that treatment is administered for the purpose of diminishing or decreasing the risk of developing the pathology further. Thus, a prophylactic treatment functions as a preventative treatment against a pathology associated with L-selectin. In particular embodiments, prophylactic treatments reduce or delay physical symptoms associated with a pathology associated with L-selectin.

A “therapeutic treatment” includes a treatment administered to a subject (e.g., the subject 106) who displays symptoms or signs of a pathology associated with L-selectin and is administered to the subject for the purpose of diminishing or eliminating those signs or symptoms of the pathology associated with L-selectin. The therapeutic treatment can reduce, control, or eliminate the presence or activity of the pathology associated with L-selectin and/or reduce control or eliminate side effects of the pathology associated with L-selectin.

Function as an effective amount, prophylactic treatment or therapeutic treatment are not mutually exclusive, and in particular embodiments, administered dosages may accomplish more than one treatment type.

In particular embodiments, therapeutically effective amounts induce a reduced progression of a pathology associated with L-selectin. In particular embodiments, the reduced progression includes reduced or stabilized symptoms, reduced or stabilized levels of one or more markers associated with the pathology, or a change in another metric known in the art.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of condition, stage of condition, previous or concurrent therapeutic interventions, idiopathy of the subject and route of administration.

Useful doses can range from 0.1 to 5 μg/kg or from 0.5 to 1 μg/kg. In other examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Exemplary doses of cell-based compositions can include 10⁴ to 10⁹ cells/kg body weight, or 10³ to 10¹¹ cells/kg body weight. Therapeutically effective amounts to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly). In particular embodiments, the treatment protocol may be dictated by a clinical trial protocol or an FDA-approved treatment protocol.

The compositions described herein can be administered by, for example, injection, inhalation, infusion, perfusion, lavage, or ingestion. Routes of administration can include intravenous, intradermal, intraarterial, intranodal, intravesicular, intrathecal, intraperitoneal, intraparenteral, intranasal, intralesional, intramuscular, oral, subcutaneous, and/or sublingual administration. Formulations are generally be administered by injection.

In various implementations of the present disclosure, the oligonucleotide 202 can be administered by, e.g., injection, infusion, perfusion, or lavage. Routes of administration can include bolus intravenous, intradermal, intraarterial, intraparenteral, intranodal, intralymphatic, intraperitoneal, intralesional, intraprostatic, intravaginal, intrarectal, topical, intrathecal, intratumoral, intramuscular, intravesical, and/or subcutaneous administration.

In some implementations, the oligonucleotide 202 is administered with a pharmaceutically acceptable carrier. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety and purity standards as required by United States FDA Office of Biological Standards and/or other relevant foreign regulatory agencies.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, chelating agents (e.g., EDTA), gels, binders, disintegration agents, and/or lubricants.

Exemplary buffering agents include citrate buffers, succinate buffers, tartrate buffers, fumarate buffers, gluconate buffers, oxalate buffers, lactate buffers, acetate buffers, phosphate buffers, histidine buffers and/or trimethylamine salts.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol and 3-pentanol.

Exemplary isotonic agents include polyhydric sugar alcohols including trihydric or higher sugar alcohols, such as glycerin, erythritol, arabitol, xylitol, sorbitol or mannitol.

Exemplary stabilizers include organic sugars, polyhydric sugar alcohols, polyethylene glycol, sulfur-containing reducing agents, amino acids, low molecular weight polypeptides, proteins, immunoglobulins, hydrophilic polymers or polysaccharides.

In some implementations, the oligonucleotide 202, the reversal construct 116, and other elements described herein can be included in one or more kits. Kits can include various components to practice methods disclosed herein. For example, kits can include the oligonucleotide 202, the reversal construct 116, L-selectin, a nucleic acid encoding L-selectin, a chimeric antigen receptor (CAR), a nucleic acid encoding a CAR, cells (e.g., immune cells, T-cells, CD62L+ T cells, naïve T cells (T_{N}), stem cell memory T cells (T_{SCM}), and central memory T cells (T_{CM}), CD4 T-cells, CD8 T-cells, B cells, natural killer (NK) cells, NK-T-cells, monocytes/macrophages, lymphocytes, hematopoietic stem cells (HSCs), hematopoietic progenitor cells (HPC), and/or a mixture of HSC and HPC (i.e., HSPC), untransduced T-cells, CAR T-cells); cell lines (e.g., J.RT3-T.35, Jurkat, and H9 cell lines); tissue samples (e.g., specimens, or other organ, and/or cells derived therefrom); genetic expression components (e.g., genes for expression provided by vectors (e.g., lentiviral vector, retroviral vector), CRISPR components, ZFNs, TALENs, MegaTALs, targeted viral vectors and/or nanoparticles); cell formulations or activation components (e.g., saline, buffered saline, phosphate buffered saline (PBS); biocompatible buffers such as, Ca++/Mg++ free PBS; physiological saline, water, Hanks' solution, Ringer's solution, T-cell stimulating epitopes (e.g., anti-CD3/anti-CD28 conjugated beads; OKT3, TGN1412), culture-initiating compositions, RPMI medium, non-essential amino acids, sodium pyruvate, penicillin/streptomycin, non-dividing EBV-transformed lymphoblastoid cells (LCL), IL-21, human serum albumin (HSA) or other human serum components or fetal bovine serum, dextrose, Stabilizers, preservatives); components for screening form KMT2A fusion (e.g., fusion probe, KMT2A probe, fluorescent-labeled nucleotide analog, microscope, FISH analytics software); combination therapy components (e.g., local anesthetics, chemotherapeutic agents, immunosuppressive agents, anti-inflammatory agents); an antibody tagged with a fluorescent molecule; PCR amplification sequences; cytokines (e.g., IL-2, IL-7, IL-15, IL-21); culture vessels; GAPDH; IFN-γ enzyme-linked immunosorbent assay (ELISA); culture plates; etc.

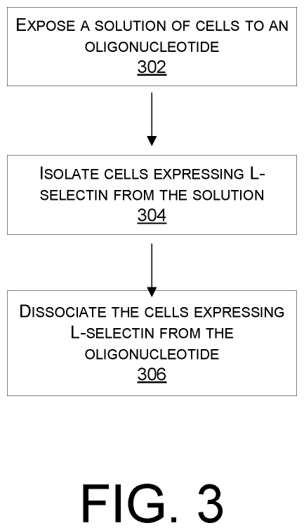

FIG. 3 illustrates an example process for isolating a cell that expresses L-selectin using an oligonucleotide that specifically binds L-selectin. The process 300 is performed by an entity, which may include one or more of a flow cytometer, a fluidic device (e.g., a microfluidic device), a computing device, or a user (e.g., a laboratory technician, care provider, or the like). According to some implementations, any of the steps of process 300 may be omitted.

At 302, the entity exposes a solution of cells to the oligonucleotide that specifically binds L-selectin. In various implementations, the solution is produced from a biological sample derived from a subject. The subject may be a human or other animal. In some examples, the subject suffers from a pathology associated with cells that express L-selectin. In some examples, the subject is a donor.

In various implementations, the solution of cells includes cells of more than one cell type. For example, the solution may include immune cells (e.g., lymphocytes, granulocytes, monocytes, etc.), red blood cells, epithelial cells, fat cells, muscle cells, and/or stem cells. In some examples, the biological sample includes T cells. The T cells may include at least one of naïve T cells (T_{N}), stem cell memory T cells (T_{SCM}), and central memory T cells (T_{CM}). The T cells may include CD62+ T cells.

In various implementations of the present disclosure, the oligonucleotide includes a sequence having at least 70%, 75%, 80%, 85%, 90%, 95%, 97%, or 99% sequence identify to SEQ ID NO: 1, SEQ ID NO: 2, SEQ ID NO: 3, SEQ ID NO: 4, SEQ ID NO: 5, or SEQ ID NO: 6.

In various implementations, the oligonucleotide is conjugated to a tag or a solid support. The tag may be a fluorophore, biotin, or a dye. In some implementations, the solid support is paper, glass, a polymer, a resin, a bead, a particle, or a microbubble.

At 304, the entity isolates the cells expressing L-selectin from the solution. In some examples, the entity isolates the cells expressing L-selectin by removing or manipulating the solid support. For example, the solid support may be a magnetic bead, and the entity may apply a magnetic field to the solution to isolate the cells expressing L-selectin. In some instances, the solid support is a microbubble, and the entity may mix the oligonucleotide conjugated to the microbubble with the solution. Based on mixing the oligonucleotide with the solution, the cells expressing L-selectin may bind to the oligonucleotide and float to the surface of the solution. In some examples, the solid support is paper, glass, a polymer, or a resin. The cells expressing L-selectin can be isolated by removing the solid support from the solution.

In various examples, the cells that do not express L-selectin can be removed by decanting, draining, aspirating, washing, or another method. In some examples, the cells expressing L-selectin are isolated from the solution, for example, by aspirating.

At 306, the entity dissociates the oligonucleotide from the cells expressing L-selectin. In various implementations, a second solution includes the oligonucleotide bound to the cells expressing L-selectin. The oligonucleotide is dissociated from the cells expressing L-selectin, in some instances, by administering a reversal construct to the second solution, changing a pH of the second solution, changing a salt concentration of the second solution, changing the temperature of the second solution, applying a mechanical force to the second solution, or administering a nuclease enzyme to the second solution.

In various implementations, the reversal construct includes a second oligonucleotide including a sequence having at least 70%, 75%, 80%, 85%, 90%, 95%, 97%, or 99% sequence identify to SEQ ID NO: 7.

### EXPERIMENTAL EXAMPLE

### Introduction

This Experimental Example describes an adaptation of the cell-SELEX method and provides a novel CD62L/L-Selectin aptamer (L-SA, for “L-Selectin Aptamer”) that binds with high affinity to both soluble L-selectin and cell-associated CD62L/L-selectin (Sefah, K., et al., Nat. Prot., 2010. 5(6): p. 1169-1185). It is demonstrated that L-SA binds to CD62L by Pull Down assay in combination with LS-MS/MS, and siRNA knockdown. As a result, 2 aptamers (binding CD8 and CD62) were combined for sequential traceless isolation of CD8+CD62L+ T cells.

The present disclosure provides the identification of a CD62L/L-Selectin aptamer that binds to both soluble and cell surface-expressed protein with nanomolar affinity. The present disclosure describes use of the aptamers for isolation of CD62L+ T cells with high purity. In addition to its application for cell therapy manufacturing, CD62L aptamers have many other potential biomedical applications. For example, bivalent aptamers containing a CD62L-binding domain with a cancer cell-targeting domain have already been shown to mediate cell-cell interaction between T cells and cancer cells (Liu, X., et al., Small, 2011. 7(12): p. 1673-1682; Yang, Y., et al., ACS nano, 2020. 14(8): p. 9562-9571). Induction of cell-cell coupling followed by T cell activation induces cancer cell killing both in vitro and in vivo (Yang, Y., et al., ACS nano, 2020. 14(8): p. 9562-9571). Since CD62L facilitates immune cell infiltration into tissues, aptamer-based constructs that block CD62L binding can reduce immune responses after organ transplantation or in diseases such as diabetes. Antibody-based blocking of CD62L has been shown to prevent insulitis and diabetes in mouse models (Yang, X.-D., et al., Proc. Natl. Acad. Sci. U.S.A., 1993. 90(22): p. 10494-10498). In addition, lymphocyte entry to renal and cardiac transplants has been shown to be mediated by L-selectin (Turunen, J. P., et al., J. Exp. Med., 1995. 182(4): p. 1133-1141; Turunen, J. P., et al., Eur. J. Immunol., 1994. 24(5): p. 1130-1136). Reducing lymphocyte extravasation with CD62L-binding aptamers could therefore help reduce rejection after organ transplantation. Since the CD62L aptamers reported in this work bind to soluble L-Selectin, the aptamers could also be used in diagnostics. Soluble L-selectin levels are altered in diseases such as lupus, sepsis, and diabetes-related coronary artery disease (Albertini, J.-P., et al., Diabetes Care, 1999. 22(12): p. 2044-2048; Font, J., et al., Clin. Exp. Immunol, 2000. 119(1): p. 169-174; Seidelin, J. B., et al., Intensive Care Med., 2002. 28(11): p. 1613-1618). The aptamers reported here could be applied as molecular recognition agents in point-of-care diagnostics for soluble L-selectin levels.

### Materials and Methods

### Cell Culture and PBMC Isolation

The J.RT3-T3.5 cell line (human T-ALL) was purchased from American Type Culture Collection (ATCC) of Manassas, Virginia. Positively selected CD4+ T cells, T cells, and T cell-depleted apheresis were provided by Juno Therapeutics of Seattle, Washington. Peripheral blood mononuclear cells (PBMCs) were isolated from Leukocyte Reduction System (LRS) chambers (Bloodworks Northwest of Seattle, Washington) by density gradient centrifugation over Ficoll-Paque (General Electric Life Sciences of Marlborough, Massachusetts). All cells in this example were cultured in RPMI 1640 medium (Gibco, now Thermo Fisher of Waltham, Massachusetts and Corning of Corning, New York) supplemented with 10% heat-inactivated fetal bovine serum (FBS) (Life Technologies or Carlsbad, California and VWR of Radnor, Pennsylvania). The CD4+ T cells used in SELEX were rested in media supplemented with 20 ng/mL recombinant human IL-2 (Miltenyi Biotec of Cologne, Germany) overnight prior to selection to remove the cell-freezing reagent, dimethyl sulfoxide (DMSO). For cell selection experiments, PBMCs were used either immediately after isolation from LRS chambers or after incubation in media for 15 hours post-thaw to restore CD62L expression.

### Oligonucleotides and Buffers

Aptamer sequences used in the study are listed in Table 1. All oligonucleotides in this example were purchased from Integrated DNA Technologies (IDT) of Coralville, Iowa. The starting library and individual aptamers were purified by high performance liquid chromatography (HPLC) and the primers were purified by a standard desalting procedure. Buffer compositions were adapted from previously published protocols (Mahnke, Y. D., et al., Eur. J. Immunol., 2013. 43(11): p. 2797-2809). The base wash buffer (WB) was prepared by further supplementing Dulbecco's phosphate-buffered saline (DPBS) with calcium and magnesium (Corning) with 5 mM MgCl₂ and 25 mM D-(+)-Glucose (Sigma-Aldrich). For the experiments in this example, aptamers were annealed at 1 μM by heating the solution at 95° C. for 5 min, followed by snap cooling on ice for at least 15 min.

TABLE 1
Aptamer Sequences.

| Name | Sequence |
|---|---|
| CD62LApt.88 | 5'-ATCCAGAGTGACGCAGCATAGTCAAGGCGATAGACGAAAAACATTGTTTTCCTG |
| | GAAGGCCCAGAATCTGTGGACACGGTGGCTTAGT-3' (SEQ ID NO: 4) |
| CD62LApt.71 | 5'-CCAGAGTGACGCAGCATAGTCAAGGCGATAGACGAAAAACATTGTTTTCCTGGA |
| | AGGCCCAGAATCTGTGG-3' (SEQ ID NO: 3) |
| CD62LApt.61 | 5'-AGACCCGACGCAGCATAGTCAAGGCGATAGACGAAAAACATTGTTTTCCTGGA |
| | AGGCCCAGAATCTGCG-3' (SEQ ID NO: 2) |
| CD62LApt.52 | 5'-TAGTCAAGGCGATAGACGAAAAACATTGTTTTCCTGGAAGGCCCAGAATCTG-3' |
| | (SEQ ID NO: 1) |
| CD62LApt.42 | 5'-CTGGGCGATAGACGAAAAACATTGTTTTCCTGGAAGGCCCAG-3' (SEQ ID NO: 6) |
| CD62LApt.52.s20 | 5'-TTTTTTTTTTTTTTTTAATTTAGTCAAGGCGATAGACGAAAAACATTGTTTTCCTG |
| | GAAGGCCCAGAATCTG-3' (SEQ ID NO: 5) |
| Random Aptamer (RAN) | 5'-ATCCAGAGTGACGCAGCAAATTCCAAACTCGAGTAAGCGTAGAGCCTCTCATC |
| | GCCTCAATAATGGACACGGTGGCTTAGT-3' (SEQ ID NO: 8) |
| Reversal Agent | 5'-CAGATTCTGGGCCTTCCAGGAAAACAAT-3' (SEQ ID NO: 7) |

Table 1 provides the sequences of aptamers use in this example. Aptamers were ordered with 6-FAM, or biotin on the 5′ end and used as described in the figures. For aptamers with a biotin modification, an 18-atom hexaethlyene glycol spacer is included between the 5′ biotin and the aptamer sequence. Underlined sequences represent the constant regions and bolded sequences represent modifications added to the aptamer.

### Competitive Cell-SELEX

Cell-SELEX protocols were adapted from previously published protocols (Mahnke, Y. D., et al., Eur. J. Immunol., 2013. 43(11): p. 2797-2809; Muroyama, Y. and E. J. Wherry, Cold Spring Harbor Perspect. Biol., 2021. 13(10): p. a037929). In round 1, 5.5 nmol of the initial library (10¹⁶ unique sequences) was annealed and incubated with 30 million T cells in 400 μL WB supplemented with 0.1% bovine serum albumin (BSA) for 1 hour at 4° C. to avoid aptamer internalization. After incubation, the cells were washed with WB to remove unbound sequences. From rounds 2-7, positive selection was performed with increasing stringency by decreasing the library concentration, decreasing the number of target cells, reducing the incubation time, increasing the number of washes, and/or increasing the concentration of protein blocker. In round 8, PBMCs were used for competitive positive selection, where the sequences are selected against the target (CD4 T cells) in the presence of competitors (non-CD4 T cells in PBMCs). Untouched T cells were then isolated by magnetic-activated cell sorting (MACS) using a Pan T Cell Isolation Kit (Miltenyi Biotec). After positive selection, bound sequences were eluted by heating the cells at 95° C. for 10 min in 400 μL molecular grade H₂O. Cell debris was pelleted by centrifugation at 13,100 times the force of gravity (×g) for 5 min at 4° C., while the eluted sequences were simultaneously re-folded. The supernatant containing eluted sequences was used for polymerase chain reaction (PCR) amplification (rounds 1 and 8) or negative selection (rounds 2-7). For negative selection, the eluted sequences were incubated with 10 million J.RT3-T3.5 cells (rounds 2-3) or T cell-depleted PBMCs (rounds 4-7) with the same protein blocker and for the same time that were used in positive selection. The sequences bound to the undesired cells were removed by centrifugation at 13,100×g for 5 min at 4° C., and the supernatant was collected. After each round, the PCR cycles were optimized to amplify the libraries from the supernatant with Phusion High-Fidelity DNA polymerase (New England Biolabs of Ipswich, Massachusetts), dNTP (QIAGEN of Hilden, Germany), FAM-labeled forward primer (IDT), and biotinylated reverse primer (IDT). To prepare single stranded DNA (ssDNA) from the PCR product, the amplicons were first captured on High Capacity Neutravidin Agarose Resin (Thermo Fisher) via the biotinylated reverse strand, and the forward strand was eluted from the resin by denaturation with 500 μL of 200 mM NaOH. The resulting ssDNA was desalted into 1 mL molecular grade water using a NAP-5 desalting column (Cytiva of Marlborough, Massachusetts), dried on a Savant ISS110 SpeedVac concentrator (Thermo Fisher), and annealed in WB at 1 μM for selection in the next round. Detailed experimental conditions in each round are summarized in Table 2.

TABLE 2
Cell SELEX Experimental Conditions

| | | Positive selection | | | | Negative selection | | |
|---|---|---|---|---|---|---|---|---|
| | | | | | Protein | | | Protein |
| Round # | Library | Cell type and # | Time | Washes | blocker | Cell type and # | Time | blocker |
| 1 | 13.7 | 30 million T cells | 1 | 3X | 0.1% BSA | None | 1 | 0.1% BSA |
| | mM | | hour | | | | hour | |
| 2 | 500 | 10 million CD4+ T | 1 | 3X | 0.1% BSA | 10 million | 1 | 0.1% BSA |
| | nM | cells | hour | | | J.RT3-T3.5 | hour | |
| 3 | 500 | 5 million CD4+ T | 0.5 | 3X | 1% BSA | 10 million | 0.5 | 1% BSA |
| | nM | cells | hours | | | J.RT3-T3.5 | hours | |
| 4 | 250 | 5 million CD4+ T | 0.5 | 3X | 1% BSA | 10 million T | 0.5 | 1% BSA |
| | nM | cells | hours | | | cell depleted | hours | |
| | | | | | | PBMC | | |
| 5 | 250 | 2.5 million CD4+ T | 0.5 | 4X | 2% BSA | 10 million T | 0.5 | 2% BSA |
| | nM | cells | hours | | | cell depleted | hours | |
| | | | | | | PBMC | | |
| 6 | 250 | 2.5 million CD4+ T | 0.5 | 4X | 5% BSA | 10 million T | 0.5 | 5% FBS |
| | nM | cells | hours | | | cell depleted | hours | |
| | | | | | | PBMC | | |
| 7 | 250 | 2.5 million CD4+ T | 0.5 | 4X | 5% BSA | 10 million T | 0.5 | 5% FBS |
| | nM | cells | hours | | | cell depleted | hours | |
| | | | | | | PBMC | | |
| 8 | 250 | 5 million PBMC and | 0.5 | 4X | 5% BSA | None | 0.5 | 5% FBS |
| | nM | T cell isolation | hours | | | | hours | |

Table 2 provides the experimental conditions for cell-SELEX in this example. PBMC: peripheral blood mononuclear cells; BSA: bovine serum albumin; FBS: fetal bovine serum.

### NGS and Sequence Analysis

The aptamer pools from each round of SELEX were PCR amplified with barcoded primers as listed in Table 3, purified by gel extraction, and sequenced using the MiSeq Reagent Kit v2 (300 cycles) on a MiSeq System (Illumina of San Diego, California). FASTAptamer v1.0.3 toolkit was used to analyze the FASTA files (Gattinoni, L., et al., Nat. Med., 2011. 17(10): p. 1290-1297). FASTAptamer-count was first used to identify top aptamers with the highest frequency (high count). FASTAptamer-enrich was then used to analyze the fold-enrichment of each unique sequence in adjacent rounds. MEME suite v5.2.0 Motif Discovery tool was used to predict binding motifs of top aptamers in final rounds based on their sequence similarities (Berger, C., et al., J. Clin. Invest., 2008. 118(1): p. 294-305). The top 50 aptamer sequences in the final round were used for phylogenic tree generation with FigTree toolkit v1.4.4 to assess the similarities among sequences (Arcangeli, S., et al., Front. Immunol., 2020. 11: p. 1217). Nucleic Acid Package (NUPACK) is a software tool for the analysis and design of nucleic acid structures, devices, and systems. The NUPACK web application was used to simulate aptamers folding into their thermally stable secondary structures (Xu, Y., et al., Blood, 2014. 123(24): p. 3750-3759).

TABLE 3
NGS primers used tor sequencing round 2-8. The barcode
of each reverse primer is underlined.

| Name | Sequence |
|---|---|
| Forward primer | AATGATACGGCGACCACCGAGATCTACACCGAGGAGATACCACTAAGCCACC |
| | GTGTCCA (SEQ ID NO: 9) |
| Reverse primer_Round 2 | CAAGCAGAAGACGGCATACGAGATTGCAGACAACAGACCGTCGATCCAGAGT |
| | GACGCAGCA (SEQ ID NO: 10) |
| Reverse primer_Round 3 | CAAGCAGAAGACGGCATACGAGATAGTTGACCACAGACCGTCGATCCAGAGT |
| | GACGCAGCA (SEQ ID NO: 11) |
| Reverse primer_Round 4 | CAAGCAGAAGACGGCATACGAGATGTCTCCTAACAGACCGTCGATCCAGAGT |
| | GACGCAGCA (SEQ ID NO: 12) |
| Reverse primer_Round 5 | CAAGCAGAAGACGGCATACGAGATCTGCAATCACAGACCGTCGATCCAGAGT |
| | GACGCAGCA (SEQ ID NO: 13) |
| Reverse primer_Round 6 | CAAGCAGAAGACGGCATACGAGATTGAGCGAAACAGACCGTCGATCCAGAGT |
| | GACGCAGCA (SEQ ID NO: 14) |
| Reverse primer_Round 7 | CAAGCAGAAGACGGCATACGAGATTTGGACTGACAGACCGTCGATCCAGAGT |
| | GACGCAGCA (SEQ ID NO: 15) |
| Reverse primer_Round 8 | CAAGCAGAAGACGGCATACGAGATAGCAATCCACAGACCGTCGATCCAGAGT |
| | GACGCAGCA (SEQ ID NO: 16) |

### Antibodies and Flow Cytometry

The following antibodies were used for flow cytometry: fluorescein isothiocyanate (FITC) anti-human CD3 (1:100, BioLegend of San Diego, California, UCHT1), phycoerythrin (PE) anti-human CD3 (1:200, BioLegend, HIT3a), allophycocyanin (APC) anti-human CD8a (1:100, BioLegend, RPA-T8), PE anti-human CD62L (1:100, Miltenyi Biotec, 145/15), Alexa Fluor 488 anti-human CD62L (1:25, BioLegend, DREG-56), PE/Cyanine7 anti-human CD45RA (1:200, BioLegend, HI100), Brilliant Violet 605 anti-human CD45RO (1:100, BioLegend, UCHL1), peridinin-chlorophyll-protein (PerCP)/Cyanine5.5 anti-human CD197 (1:50, BioLegend, G0431H7), FITC anti-human CD14 (0 APC-Cy7-antihuman CD14 (1:200, Molecular probes of Eugene, Oregon, 61D3), Super Bright 702-antihuman CD19 (1:100, eBioscience, now Thermo Fisher, SJ25C1), Super Bright 600-antihuman CD56 (1:100, eBioscience, TULY56), FITC-antihuman CD8a (1:100, BioLegend, RPA-T8).

### Binding Assays

Cells were washed with DPBS without calcium and magnesium (Gibco) and incubated with Zombie Violet (1:500, 10⁷ cells/mL, BioLegend) in DPBS without calcium and magnesium for live/dead staining for 15 min at room temperature. After live/dead staining, cells were washed with WB supplemented with 1% BSA to quench the remaining dye and aliquoted in a 96-well plate at 1-2×10⁵ cells/well for immortalized cells and 5-10×10⁵ cells/well for PBMCs. Prior to staining with antibodies, cells were first blocked with 10 μL/well FcR Blocking Reagent (Miltenyi Biotec) for 10 min at 4° C. For antibody staining without aptamers, cells were stained with antibodies diluted in 100 μL WB supplemented with 1% BSA for 20-30 min at 4° C. For aptamer staining without antibodies, cells were stained with annealed aptamer pools or individual aptamers at the indicated concentrations in 100 μL binding buffer (WB supplemented with 1-2% BSA and 0.1 mg/mL salmon sperm DNA (Invitrogen of Waltham, Massachusetts) and/or 0.1 mg/mL yeast tRNA (Invitrogen)) for 20-30 min at 4° C. For antibody co-staining and competition experiments, CD62L antibody (clone DREG-56 or 145/15) was added together with fluorescently labeled aptamer at the indicated concentrations. For staining cells with biotinylated aptamers, secondary staining with Streptavidin-AF647 (1:500, BioLegend) was performed after the washing steps in 100 μL binding buffer for 15 min at 4° C. For reversal agent (RA) testing, cells were first stained with 10 nM biotinylated aptamers, washed twice with 200 μL WB supplemented with 1% BSA, and then incubated with the RA (pre-warmed at 37° C.) in 100 μL WB supplemented with 0.5% BSA at the indicated concentrations for 10 min at room temperature. The cells were washed twice with 200 μL WB supplemented with 1% BSA to remove released aptamers and excess RA. Finally, the cells were stained with Streptavidin-AF647 in 100 μL binding buffer for 15 min at 4° C.

After staining, cells were washed twice with 200 μL WB supplemented with 1% BSA, fixed in WB supplemented with 1% BSA and 0.2% paraformaldehyde (PFA), and analyzed on an Attune NxT Cytometer (Invitrogen).

### Membrane Protein Extraction

Protocols for extracting membrane proteins and performing aptamer-based pulldowns were modified from previously published protocols (Sommermeyer, D., et al., Leukemia, 2016. 30(2): p. 492-500.; Fraietta, J. A., et al., Nat. Med., 2018. 24(5): p. 563-571). 240×10⁶ J.RT3-T3.5 cells were washed 3 times with DPBS and split into two groups, each containing 120×10⁶ cells. The cells were lysed with 5 mL hypotonic buffer comprised of 10 mM Tris-HCl pH7.5 supplemented with ethylenediaminetetraacetic acid (EDTA)-free cOmplete Protease Inhibitor Cocktail (Roche of Basel, Switzerland) and 1 mM phenylmethylsulfonyl fluoride (PMSF) (Thermo Fisher) for 30 min at 4° C. with end-over-end mixing. Cell membrane debris was pelleted by centrifuging the cell suspension at 16000×g for 15 min at 4° C., after which the pellet was washed 3 times with 5 mL hypotonic buffer to remove intracellular proteins. To extract and solubilize membrane proteins, the membrane pellet was resuspended in 1 mL WB supplemented with 1% Triton X-100, protease inhibitor cocktail, and 1 mM PMSF and incubated for 30 min at 4° C. with end-over-end mixing. The mixture was sonicated for 5 min in an ice water bath and pelleted at 16000×g for 15 min at 4° C. The supernatant containing solubilized membrane proteins was collected, snap frozen on dry ice for 10 min, and stored at −80° C. before use.

### Aptamer-Based Pulldown

1 mL thawed protein extracts were spiked with 100 nM Non-structural protein 1 (NS)-biotin and supplemented with 0.1 mg/mL yeast transfer RNA (tRNA) (Invitrogen) and incubated at 4° C. for 30 min with end-over-end mixing to pre-clear the extract. 2 mg washed DYNABEADS™ MyOne™ Streptavidin C1 (Invitrogen) were added to the mixture and incubated for an additional 15 min to magnetically remove proteins bound to NS-biotin. For the control group, 1.5 mg washed DYNABEADS™ MyOne™ Streptavidin C1 were first saturated with 50 nmol biotin for 15 min at 4° C., then added to the pre-cleared extract and incubated for 30 min at 4° C. with end-over-end mixing. For the aptamer group, the pre-cleared extract was spiked with 100 nM biotinylated L-SA.88 and supplemented with 0.1 mg/mL salmon sperm DNA (Invitrogen) and incubated at 4° C. for 30 min with end-over-end mixing. 1.5 mg washed DYNABEADS™ MyOne™ Streptavidin C1 were added to the mixture and incubated for an additional 15 min at 4° C. to capture L-SA.88-bound proteins. After incubation, beads in both groups were washed 5 times in 1 mL WB with 0.01% Triton X for 5 min at 4° C. with end-over-end mixing. To mildly elute the proteins, the beads were resuspended in 50 μL proteolytic digestion-compatible elution buffer composed of 5 M urea, 20 mM Tris pH 7.5, 10 mM EDTA and 100 mM Sodium Chloride (NaCl) and heated at 37° C. for 10 min followed by an additional 15 min at 47° C. The eluted proteins were stored at −80° C.

### SDS-PAGE

10 μL of the elution was combined with 10 μL 2× Laemmli sample buffer (Bio-Rad of Hercules, California) and heated at 47° C. to denature the proteins. The proteins were separated by sodium dodecyl-sulfate polyacrylamide gel electrophoresis (SDS-PAGE) on a Novex WedgeWell 4-20% Tris-Glycine gel (Invitrogen) according to the manufacturer's instructions. The gel was stained with a Colloidal Blue Staining Kit (Invitrogen) and imaged on a Gel Doc EZ system (Bio-Rad).

### Receptor Identification

40 μL of the elution from both the control group and the aptamer group were submitted to the Proteomics/Metabolomics Shared Resources at Fred Hutchinson Cancer Center of Seattle, Washington. The samples were processed and analyzed by liquid chromatography-tandem mass spectrometry (LC-MS/MS). Proteins were identified by searching the data against a Uniprot Human database along with common contaminants. The search results were filtered to only include identifications from peptides with a false discovery rate of 1% or less.

### Biolayer Interferometry (BLI)

Prior to running, Octet streptavidin (SA) biosensors (Sartorius of Göttingen, Germany) were pre-soaked in 200 μL diluent (WB supplemented with 1% BSA, 0.1 mg/mL yeast tRNA, 0.1 mg/mL salmon sperm DNA and 0.01% Tween-20). For BLI, the SA biosensors were first rinsed in 200 μL diluent for 100 sec to establish the first baseline. The rinsed sensors were transferred to wells containing 50 nM biotinylated aptamers in 200 μL diluent for aptamer immobilization onto the SA biosensors until a threshold of 0.5 nm is reached. Next, aptamer-immobilized SA biosensors were rinsed in 200 μL diluent for 100 sec to remove excess aptamers and for an additional 100 sec in 200 μL diluent to establish the second baseline. The sensors were then transferred to wells containing different concentrations of L-selectin in 200 μL diluent to allow aptamer association with CD62L (R&D Systems of Minneapolis, Minnesota), CD62P (ACROBiosystems of Newark, Delaware), or CD62E (ACROBiosystems). Finally, the sensors were transferred to wells containing 200 μL diluent to allow dissociation. The association and dissociation durations are indicated on the BLI curves.

siRNA Knockdown

5×10⁶ J.RT3-T3.5 cells in log phase growth were washed with electroporation (EP) buffer (MaxCyte of Rockville, Maryland) and mixed with 5 pmol each of CD62L siRNA 1 and 2 (10 pmol total) or 250 pmol control siRNA in 50 μL EP buffer. Table 4 illustrates the sequences of siRNA duplexes used to knockdown CD62L expression. The sample was then loaded in a R-50×3 processing assembly (MaxCyte) and electroporated using the Jurkat protocol on the ExPERT GTx system (MaxCyte). Electroporated cells were stained with 50 nM CD62L antibody (clone DREG-56) or CD62LApt.88 aptamer in 100 μL binding buffer at 24 hr and 48 hr post-electroporation, washed, fixed, and analyzed by flow cytometry.

TABLE 4
Sequences of siRNA duplexes used
to knockdown CD62L expression.

| Name | Sequence |
|---|---|
| hs.Ri.SELL.13.1-SEQ1 | 5′-rCrCrArArCrArArArUrC |
| | rUrCrUrUrArCrUrGrArArGr |
| | ArAGC-3′ (SEQ ID NO: 17) |
| hs.Ri.SELL. 13.1-SEQ2 | 5′-rGrCrUrUrCrUrUrCrAr |
| | GrUrArArGrArGrArUrUrUr |
| | GrUrUrGrGrUrU-3′ |
| | (SEQ ID NO: 18) |
| hs.Ri.SELL. 13.2-SEQ1 | 5′-rGrCrArGrUrCrArUrGr |
| | GrUrUrArCrUrGrCrArUrUr |
| | CrUrCTG-3′ |
| | (SEQ ID NO: 19) |
| hs.Ri.SELL.13.2-SEQ2 | 5′-rCrArGrArGrArArUrGr |
| | CrArGrUrArArCrCrArUrGr |
| | ArCrUrGrCrCrA-3′ |
| | (SEQ ID NO: 20) |

### Selectin of CD62L+CD8+ T Cells

Frozen PBMCs were thawed and rested in RPMI medium with 10% FBS at 5×10⁶ cells/mL for 15 hours to recover CD62L expression. CD8⁺ T cells were tracelessly selected from 100×10⁶ PBMCs as described previously in Kacherovsky, N., et al., Nat. Biomed. Eng., 2019. 3(10): p. 783-795. Tracelessly selected cells were immediately centrifuged at 500×g for 3 min at 4° C. to remove the supernatant containing the reversal agent and EDTA. In parallel, CD8⁺ T cells were selected from 100×10⁶ PBMCs of the same donor using a CD8⁺ T Cell Isolation Kit (Miltenyi Biotec) according to the manufacturer's instructions. Aptamer-immobilized MicroBeads were prepared by incubating 10 nM biotinylated CD62LApt.52.s20 aptamers with 10 μL Anti-Biotin Microbeads (Miltenyi Biotec) in 100 μL binding buffer composed of WB supplemented with 1% BSA and 0.1 mg/mL yeast tRNA for 15 min at 4° C. with end-over-end rotation. To isolate CD62L+CD8+ cells, 10×10⁶ tracelessly isolated CD8+ cells were incubated with 100 μL of the aptamer-bead solution for 20 min at 4° C. with end-over-end rotation. The suspension was centrifuged at 500×g for 3 min at 4° C. and the supernatant was removed. The cells were washed with 1 mL WB supplemented with 0.5% BSA, spun down, and resuspended in 500 μL WB supplemented with 0.5% BSA. The suspension was applied to a washed MS column mounted on a MiniMACS separator (Miltenyi Biotec) according to the manufacturer's instructions. The flow through containing unlabeled cells was collected, and the column was washed 2 times with 500 μL DPBS supplemented with 0.5% BSA and 1 time with 500 μL DPBS supplemented with 2 mM EDTA and 0.5% BSA to collect unlabeled cells. To elute the CD62L⁺CD8⁺ cells from the column, 1 mL of pre-warmed RA solution (1 μM RA in 1 mL DPBS supplemented with 2 mM EDTA, 0.5% BSA and 5 mM MgCl₂ warmed at 37° C.) was applied onto the column. The column volume (250 μL) was allowed to drain, and the column was plugged with a Male/Female (M/F) Luer Lock Plug and let incubated for 10 min at room temperature. After incubation, 400 μL was allowed to drain and the column was plugged again for a second 10-min incubation with the remaining RA solution. The remaining volume (350 μL) was drained after incubation, and the column was washed 3 times with 500 μL DPBS supplemented with 5 mM EDTA and 0.5% BSA to collect eluted cells. The washed column was removed from the separator and the column-bound cells were collected by flushing with 1 mL DPBS supplemented with 2 mM EDTA and 0.5% BSA using the provided plunger. In parallel, 10×10⁶ negatively selected CD8⁺ cells were subjected to CD62L selection with CD62L MicroBeads (Miltenyi Biotec) according to the manufacturer's instructions. The yield, purity and phenotype of the isolated cells were evaluated by flow cytometry after staining for CD62L, CD8, CD3, CD45RA, CD45RO, and CCR7 expression.

### Results and Discussion

### L-Selection Aptamer Identification

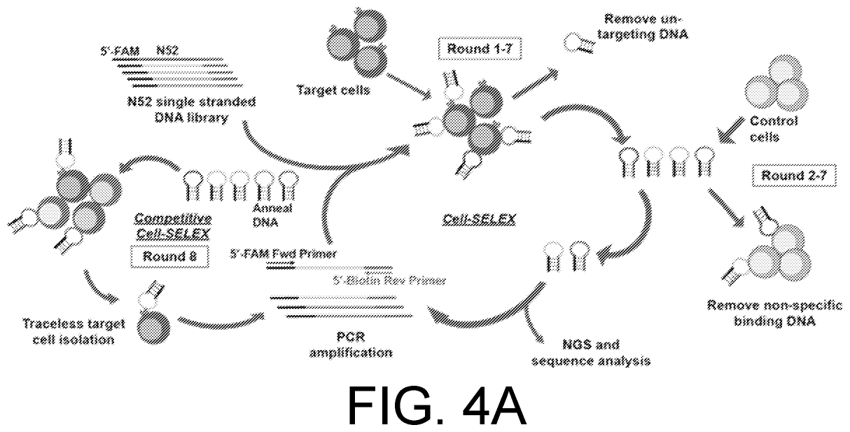

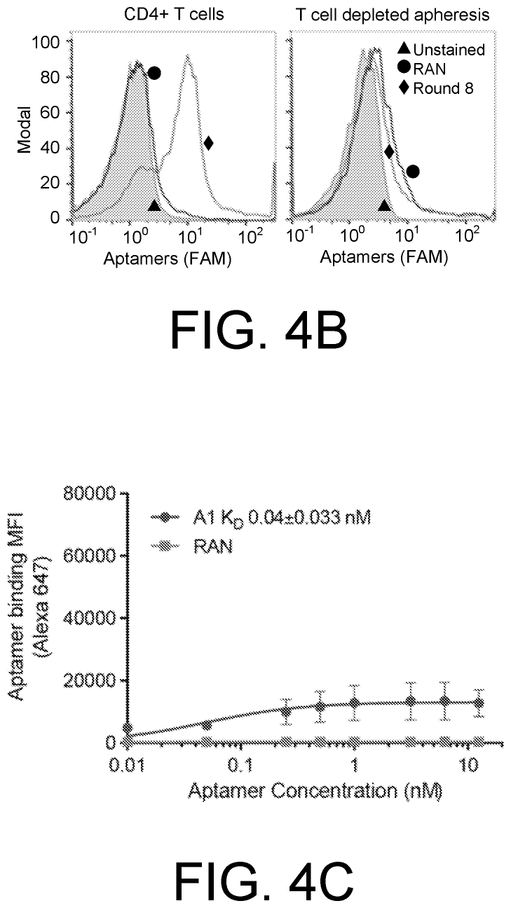

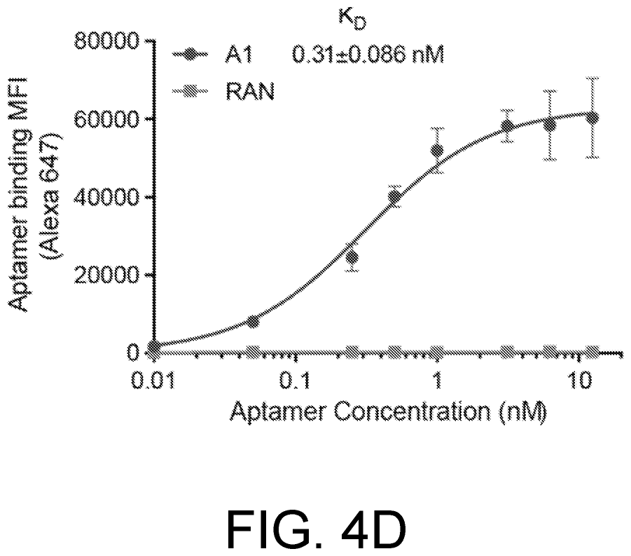

Cell-SELEX was conducted to identify T cell-binding aptamers by using human, primary CD4+ T cells for positive selection and either J.RT3-T3.5 cells, which are CD3− and CD28−, or T cell-depleted apheresis product as cells for negative selection (FIG. 4A). In the last round of selection, competitive cell-SELEX was used by incubating the Round 7 library with PBMC and then isolating T cells and associated aptamers by magnetic separation. Since the disclosed library is fluorescein-labeled, selection progress was monitored by flow cytometry (FIGS. 4A-4D). Binding of the disclosed Round 8 (R8) library to CD4+ T cells but not to T cell-depleted apheresis product was observed, indicating enrichment of aptamers binding to CD4+ T cells (FIG. 4B). A random aptamer sequence did not bind to either cell type.

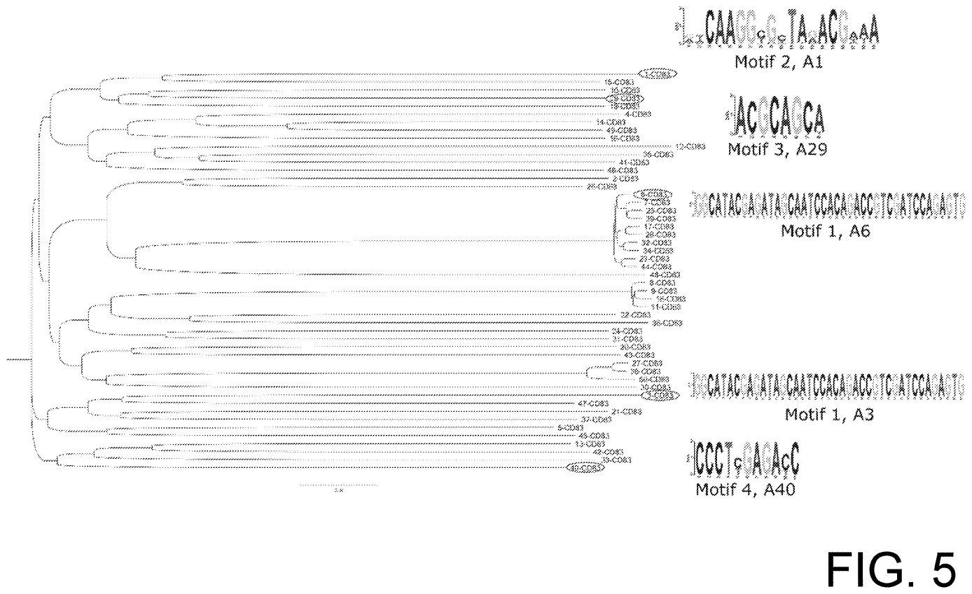

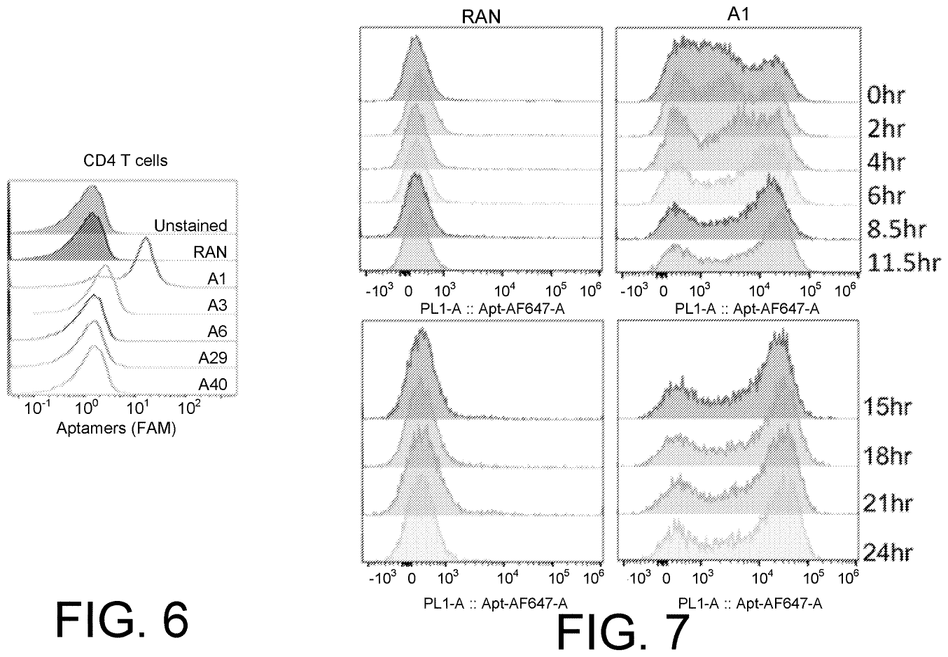

To identify individual aptamer sequences, DNA libraries from rounds 1 to 8 were sequences using next generation sequencing (NGS). The FASTAptamer tool kit was used to analyze the abundance and enrichment of unique sequences between rounds (Table 5). The top 50 most prevalent sequences in the R8 library were used for motif prediction and phylogenic tree generation. Four different binding motifs were discovered using the MEME toolkit (Bing, T., et al., Mol Cell Proteomics 2015, 14 (10), 2692-700). After generating a phylogenic tree with the FigTree software (Yang, Y., et al., ACS nano, 2020. 14(8): p. 9562-9571), five sequences with high count and high enrichment were selected from the final pool on different branches of the phylogenic tree to ensure sequence diversity among the candidate aptamers (FIG. 5). The selected aptamers, A1, A3, A6, A29, and A40 (numbered by their rank of abundance in the last round), have different predicted binding motifs as identified with the MEME toolkit (A1 with motif 2; A3 and A6 with motif 1; A29 with motif 3; A40 with motif 4). Despite the high representation of motif 1 in the final library, A3 and A6, along with A29 and A40, did not show binding to CD4+ T cells in this example (FIG. 6). A1 showed binding to most, albeit not all, CD4+ T cells that were cultured in media (FIG. 6). Therefore, the A1 sequence was characterized.

TABLE 5
Sequence Enrichment of the Sequences Found in the Final Round Library

| | | Enrichment (y/x) | | | | | |
|---|---|---|---|---|---|---|---|
| | Random Region Sequence | 8/7 | 7/6 | 6/5 | 5/4 | 4/3 | 3/2 |
| 1 | TAGTCAAGGCGATAGACGAAAAACA | 0.253651 | 5.484815 | 0.023331 | 1.641432 | 1.274672 | 9.766447 |
| | TTGTTTTCCTGGAAGGCCCAGAATC | | | | | | |
| | TG (SEQ ID NO: 21) | | | | | | |
| 2 | AGGCGCTAGACGCAAACACTTAAGC | 0.352721 | 1.764231 | 0.027676 | 1.467998 | 2.763489 | |
| | ACCATGAGTGCCAAGTTTTCCTAAT | | | | | | |
| | GG (SEQ ID NO: 22) | | | | | | |
| 3 | GACGGCATACGAGATAGCAATCCAC | | | | | | |
| | AGACCGTCGATCCAGAGTGACGCAG | | | | | | |
| | CA (SEQ ID NO: 23) | | | | | | |
| 4 | CAGAGTGACGCAGCAAATTTCAAGG | 0.569159 | 1.966454 | 0.061576 | 6.691959 | 0.656506 | 6.625616 |
| | TGCTAAACGAAATTAATATAGATGG | | | | | | |
| | GC (SEQ ID NO: 24) | | | | | | |
| 5 | CGCAGCAATCACAGGGCTACAAGGT | 0.695195 | 1.190388 | 0.049963 | 1.964697 | 12.39286 | 1.12 |
| | GCTAAACGTAAACTAGCAAGAGAAC | | | | | | |
| | TA (SEQ ID NO: 25) | | | | | | |
| 6 | CGGCATACGAGATAGCAATCCACAG | | | | | | |
| | ACCGTCGATCCAGAGTGACGCAGCA | | | | | | |
| | CA (SEQ ID NO: 26) | | | | | | |
| 7 | CGGCATACGAGATAGCAATCCACAG | | | | | | |
| | ACCGTCGATCCAGAGTGACGCAGCA | | | | | | |
| | TA (SEQ ID NO: 27) | | | | | | |
| 8 | ACGGCATACGAGATAGCAATCCACA | | | | | | |
| | GACCGTCGATCCAGAGTGACGCAGC | | | | | | |
| | AA (SEQ ID NO: 28) | | | | | | |
| 9 | ACGGCATACGAGATAGCAATCCACA | | | | | | |
| | GACCGTCGATCCAGAGTGACGCAGC | | | | | | |
| | AC (SEQ ID NO: 29) | | | | | | |
| 10 | TGTCTACGCAGTTGAACATCAAAAC | 6.504721 | 2.728603 | 0.336247 | 0.092392 | 8.366071 | 0.552502 |
| | TACAACGAGTAACTCTCCAGTTCCT | | | | | | |
| | CC (SEQ ID NO: 30) | | | | | | |
| 11 | ACGGCATACGAGATAGCAATCCACA | | | | | | |
| | GACCGTCGATCCAGAGTGACGCAGC | | | | | | |
| | AT (SEQ ID NO: 31) | | | | | | |
| 12 | ACAGAGGTGTAGAAGTACACGTGAA | 12.79541 | 0.765658 | 1.158526 | | | 1.12 |
| | CAAGCTTGAAATTGTCTCTGACAGA | | | | | | |
| | GG (SEQ ID NO: 32) | | | | | | |
| 13 | AGACGGCAACCGACGCTGACAGGTC | 0.447044 | 0.858488 | 0.026859 | 3.363997 | | |
| | AAGGTCCTTAACGATACGAATAGCC | | | | | | |
| | CA (SEQ ID NO: 33) | | | | | | |
| 14 | CGGCCCCAAGCATCAAGGCGTTAGA | 0.466722 | 2.110904 | 0.022917 | 1.201268 | 1.877976 | |
| | CGAAACCCAGGGTTCACGTAGTGTA | | | | | | |
| | AT (SEQ ID NO: 34) | | | | | | |
| 15 | CGGTCAAGGCGTTAGACGAAAGGAT | 0.398002 | 1.132631 | 0.007195 | 1.309798 | 12.39286 | |
| | TAAAAACTTCTGCTCAGCAGCCTGA | | | | | | |
| | GC (SEQ ID NO: 35) | | | | | | |
| 16 | ATAAGTACGCAGTCAAGGCGATAGA | 0.374672 | 1.287154 | 0.053843 | 2.161648 | | |
| | CGAAAAGAACACTTTCCCGGAAGGG | | | | | | |
| | CC (SEQ ID NO: 36) | | | | | | |
| 17 | CGGCATACGAGATAGCAATCCACAG | | | | | | |
| | ACCGTCGATCCAGAGTGACGCAGCA | | | | | | |
| | GA (SEQ ID NO: 37) | | | | | | |
| 18 | ACGGCATACGAGATAGCAATCCACA | | | | | | |
| | GACCGTCGATCCAGAGTGACGCAGC | | | | | | |
| | AG (SEQ ID NO: 38) | | | | | | |
| 19 | AACCCAAGCATAAGCAAGTAAAAAA | 0.693557 | 1.291736 | 0.036417 | 7.214286 | 0.5625 | |
| | TAAGACCAAGGTGCTAAACGTAATT | | | | | | |
| | AT (SEQ ID NO: 39) | | | | | | |
| 20 | GCATGCAATGGAATCAAGGCGGTAG | 0.695579 | 1.086606 | 0.087401 | 2.402379 | | |
| | ACGAAAGCACAAAACGCTTCATATT | | | | | | |
| | TC (SEQ ID NO: 40) | | | | | | |
| 21 | CAAATCCCTCGAGACCATGATTTAC | 2.738527 | 0.430243 | 0.737007 | 2.490063 | 246.6518 | |
| | GGTGACCGTCATCAATAACTCCATC | | | | | | |
| | CC (SEQ ID NO: 41) | | | | | | |
| 22 | AAATGCTGACAAGGCGCTAGACGTA | 0.376161 | 1.463173 | 0.12258 | 1.2 | 1.502976 | |
| | ACATAGAAATGAAATGTCAATGTTC | | | | | | |
| | AC (SEQ ID NO: 42) | | | | | | |
| 23 | CGGCATACGAGATAGCAATCCACAG | | | | | | |
| | ACCGTCGATCCAGAGTGACGCAGCA | | | | | | |
| | AA (SEQ ID NO: 43) | | | | | | |
| 24 | CAGCATCAAGGCGTTAGACGATAAT | 0.604331 | 1.424656 | 0.105181 | 2.806878 | 3.375 | |
| | ATGACTTACCCAAGAGGGTGCTGTA | | | | | | |
| | AC (SEQ ID NO: 44) | | | | | | |
| 25 | CGGCATACGAGATAGCAATCCACAG | | | | | | |
| | ACCGTCGATCCAGAGTGACGCAGCA | | | | | | |
| | TC (SEQ ID NO: 45) | | | | | | |
| 26 | AGGCGCTAGACGCTACCCTCCAGGT | 0.926012 | 1.360125 | | 2.806878 | | |
| | ATTCATAGGAAAAGTACCTCTATTT | | | | | | |
| | AA (SEQ ID NO: 46) | | | | | | |
| 27 | GGCATACGAGATAGCAATCCACAGA | | | | | | |
| | CCGTCGATCCAGAGTGACGCAGCAA | | | | | | |
| | TA (SEQ ID NO: 47) | | | | | | |
| 28 | CGGCATACGAGATAGCAATCCACAG | | | | | | |
| | ACCGTCGATCCAGAGTGACGCAGCA | | | | | | |
| | GT (SEQ ID NO: 48) | | | | | | |
| 29 | TGACGCAGCATGTCAACGGAAAAAT | 1.405731 | 0.462795 | 0.610232 | 0.482286 | 503.2214 | |
| | CTCTGCGGCACCATTTCGGAGGTTT | | | | | | |
| | GT (SEQ ID NO: 49) | | | | | | |
| 30 | TCGATCCAGAGTGACGCAGCATCCC | 2.415584 | 1.309994 | 0.768204 | 3.904196 | 27.02679 | |
| | TCGAGACCTGATGCTCTCGCTCTAA | | | | | | |
| | CT (SEQ ID NO: 50) | | | | | | |
| 31 | ACAGGTCAAGGCGCTAGACGAAGGA | 0.438086 | 1.375154 | 0.065175 | 1.681458 | 5.633929 | |
| | AATCTCCTCCATACGGCAATGTTGA | | | | | | |
| | TT (SEQ ID NO: 51) | | | | | | |

Table 5 illustrates sequence enrichment of the top 31 sequences found in the final round library. Enrichment is calculated as y/x, with y being the sequence count in later rounds and x being the sequence count in earlier rounds. Crossed off enrichment calculations indicate sequences were not found in the earlier rounds.

### Characterization of A1 Binding to Mammalian Cells

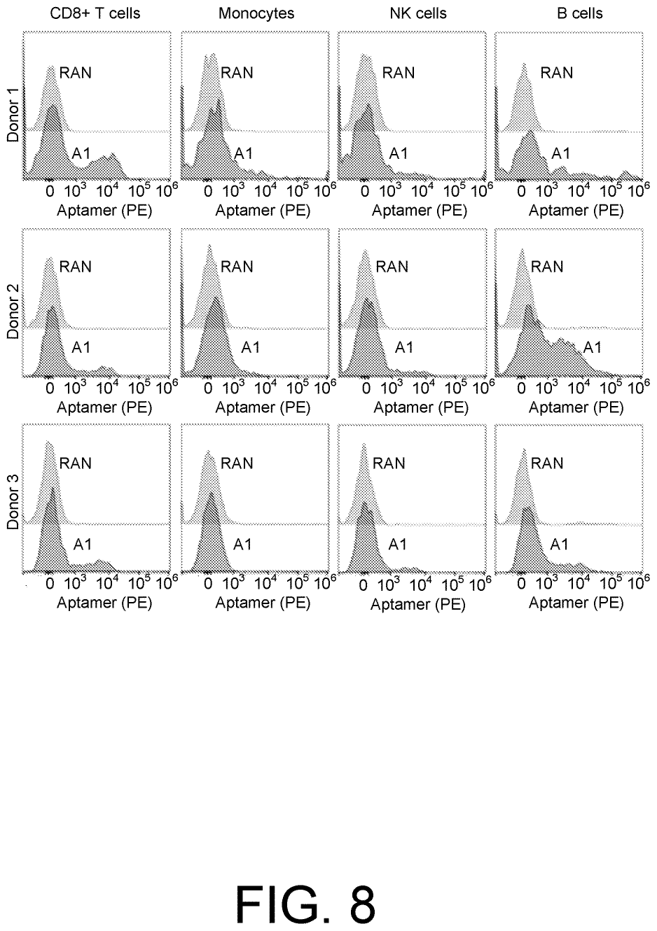

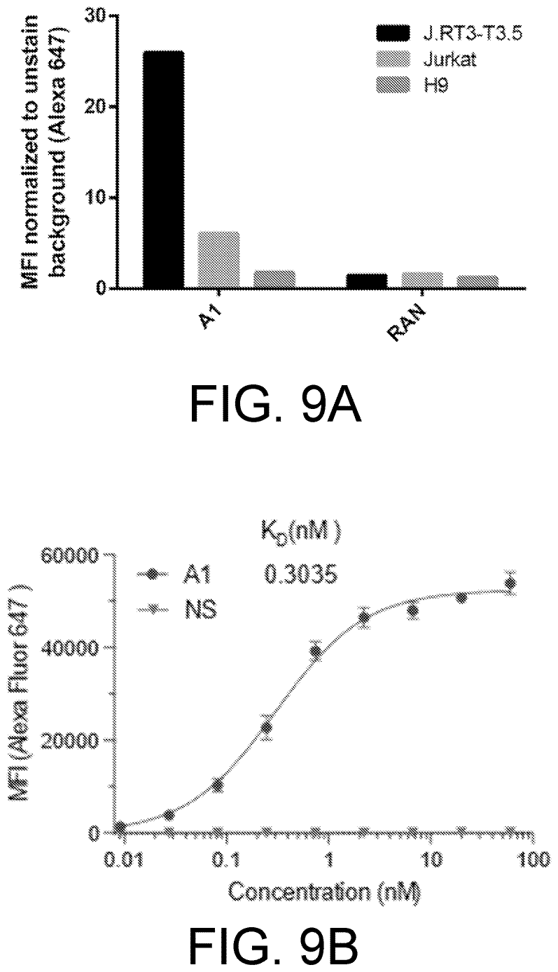

The affinity of A1 binding to its target cells was evaluated using flow cytometry. The apparent K_{D}'s of A1 binding to cryopreserved CD4⁺ T cells used immediately after thawing without culturing and to CD4⁺ T cells cultured in RPMI media with 10% FBS were, in this example, 0.04±0.033 nM and 0.31±0.086 nM, respectively (FIGS. 4C, 4D). The saturated A1 binding to cells without prior incubation was approximately a third of the saturated binding to incubated cells. A time-course experiment showed that while A1 binds only a minor subfraction of CD4⁺ T cells thawed after cryopreservation, the fraction of cells bound to aptamer increased with time in culture. The increase in binding started about 4 hours after incubating cells in RPMI 10% FBS and maximum binding was observed at around 15 hours (FIG. 7). In addition to CD4⁺ T cells, A1 shows binding to a sub-population of CD8⁺ T cells, B cells and NK cells in PBMCs (FIG. 8). Despite being used in initial selection rounds as a negative selection cell source, A1 also showed high levels of binding to J.RT3-T3.5 cells (FIG. 9A) with sub-nanomolar affinity (FIG. 9B). Regardless, J.RT3-T3.5 cells were therefore used for receptor identification due to their ease of culture and rapid expansion compared to primary CD4⁺ T cells.

### Receptor Identification and Validation

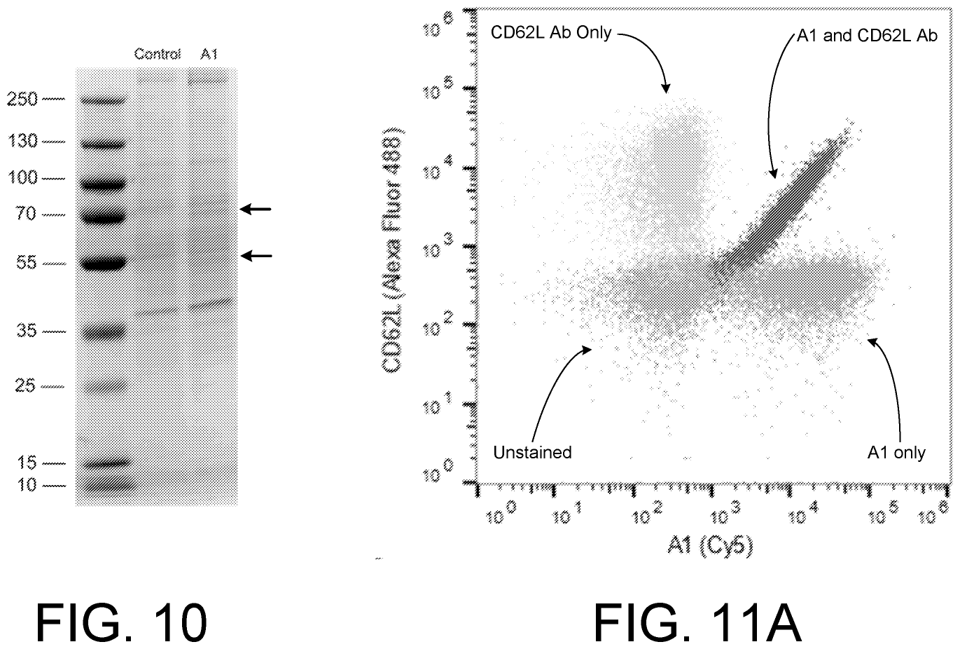

To identify the binding partner of A1, aptamer-based pulldown was performed as described in Cheng, E. L., et al., J. Am. Chem. Soc., 2022. 144(30): p. 13851-13864. Briefly, membrane proteins were extracted from JRT3 cells and pre-cleared with a non-specific aptamer. Biotinylated A1 was used to pull down target proteins from the pre-cleared protein extract, and the aptamer-protein complex was isolated using streptavidin-coupled DYNABEADS™. Proteins were eluted from the beads and analyzed by LC-MS/MS. In parallel, the eluted proteins were separated by SDS-PAGE and visualized by Colloidal Blue staining. In parallel, the eluted proteins were separated by SDS-PAGE and visualized by Colloidal Blue staining (FIG. 10). In contrast to most previously reported methods which analyze only excised protein bands after SDS-PAGE, all eluted proteins were analyzed in this example to identify proteins that are not well separated by SDS-PAGE or have a relatively low abundance even after enrichment. To identify the target from the mass spectrometry results, screened was conducted for proteins that are 1) uniquely identified in the aptamer sample and not the control sample and 2) localized to the cell membrane. Among the potential protein receptors identified by LC-MS/MS, CD62L/L-selectin was of particular interest because it has been reported that CD62L expression is lost from PBMCs, including naïve and central memory T cells, upon cryopreservation and then it recovers with time in culture post-thaw (Costantini, A., et al., J. Immunol. Methods, 2003. 278(1-2): p. 145-155; Florek, M., et al., PLoS One, 2015. 10(12): p. e0145763; Priesner, C., et al., Hum. Gene Ther., 2016. 27(10): p. 860-869; Reimann, K. A., et al., Clin. Diagn. Lab. Immunol., 2000. 7(3): p. 352-359), which is consistent with the observation that A1 binding to thawed CD4+ T cells increases with time in culture. Additionally, the observation that A1 binds to various leukocyte populations is also consistent with CD62L's broad expression on circulating lymphocytes, B cells, and polymorphonuclear granulocytes (Rainer, T. H., Resuscitation 2002, 52 (2), 127-141).

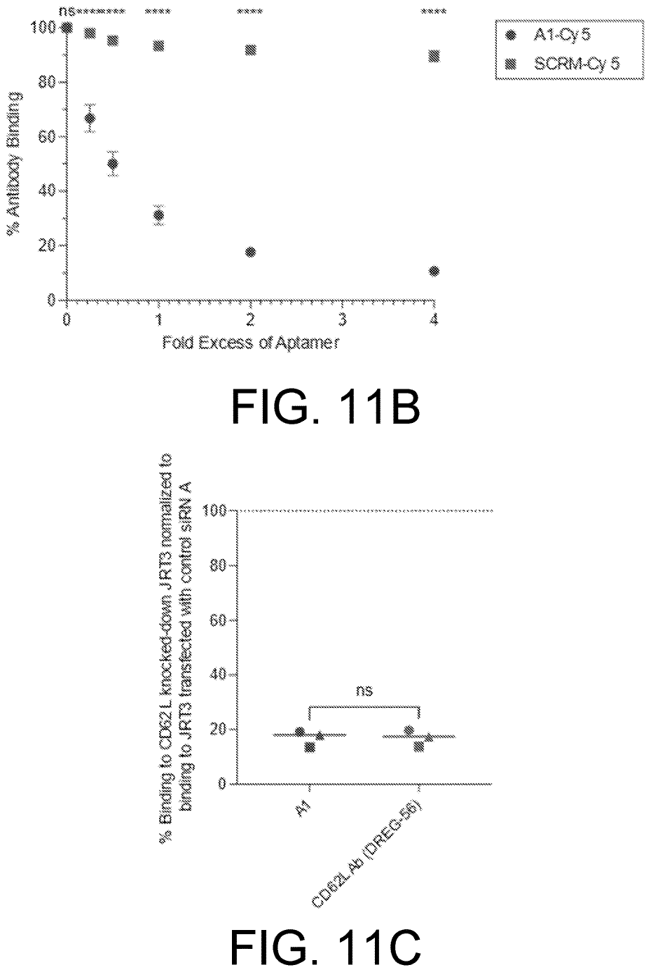

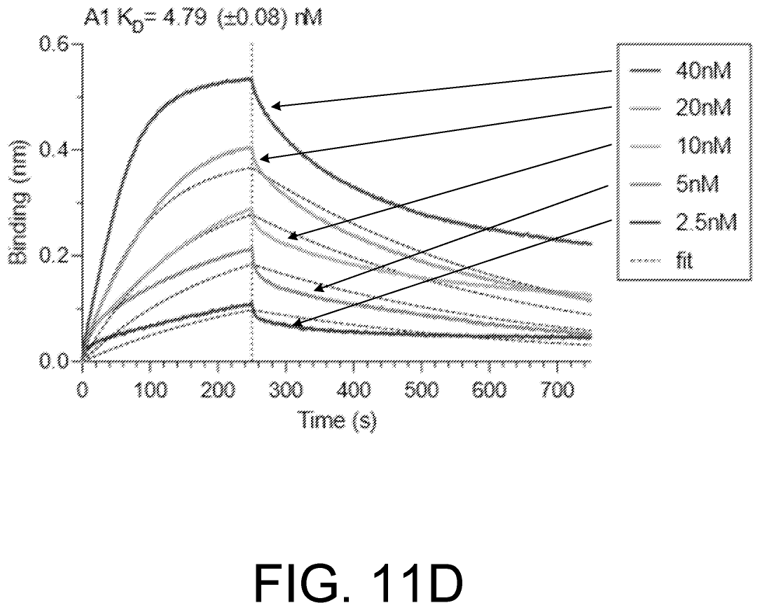

To assess A1 binding to CD62L, JRT3 cells were co-stained with A1 and a monoclonal CD62L antibody (clone DREG-56). A strong, positive correlation between L-SA.88 and the antibody was observed in this example, indicating that the aptamer binds to the same receptor as the antibody (FIG. 11A).

Next, J.RT3-T3.5 cells were incubated with a fixed concentration of the CD62L antibody (clone DREG-56) and varying concentrations of A1 to assess competition between the antibody and the aptamer (FIG. 11B). In this example, the DREG-56 antibody binding to J.RT3-T3.5 cells is reduced to 30% when A1 was added at the same concentration, and further reduced to 10% when A1 was added at 4-fold molar excess, indicating that A1 and the DREG-56 antibody have a shared binding site on CD62L, and that A1 binds to CD62L with a higher affinity than the antibody. Hicke and co-authors previously reported three L-selectin aptamers (LD174, LD196, and LD201), identified by protein-SELEX against an L-Selectin-IgG fusion protein, that also compete with DREG-56 for binding to L-Selectin (Hicke, B. J., et al., J. Clin. Invest., 1996. 98(12): p. 2688-2692). A separate aptamer (sgc3) discovered via cell-SELEX against the CCRF-CEM cell line was also reported to compete with DREG-56 for binding to L-Selectin (Shangguan, D., et al., Proc Natl Acad Sci USA 2006, 103 (32), 11838-43; Bing, T., et al., Mol Cell Proteomics 2015, 14 (10), 2692-700). While sequence homology between A1 and LD174, LD196, LD201 and sgc3 was not observed, the aptamers may bind at a similar location on CD62L.

To further validate A1 binding to CD62L, the expression of CD62L was knocked down in J.RT3-T3.5 cells with siRNA duplexes targeting the SELL gene, which encodes CD62L, and compared aptamer and antibody binding to J.RT3-T3.5 cells transfected with the SELL siRNA versus a control siRNA. At 48 hours post electroporation with CD62L siRNA, the relative aptamer binding was reduced to 16.92% in this example, while relative antibody binding was similarly reduced to 17% (FIG. 11C), thus confirming CD62L/L-Selectin as a binding partner of A1. A1 was therefore renamed CD62LApt.88 (88 denotes the length of the aptamer in nucleotides). Biolayer interferometry (BLI) was performed to interrogate the binding kinetics of CD62LApt.88 to soluble L-selectin. The protein was serially diluted, and the association and dissociation kinetics of biotinylated CD62LApt.88 immobilized on streptavidin tips was measured. The dissociation constants (K_{D}) were calculated by performing a global fit on the curves using a 1:1 model. In this example, CD62LApt.88 binds to recombinant human CD62L protein with a K_{D} of 4.91 nM.

Aptamer Truncations and Comparison with Previously Reported CD62L Aptamers

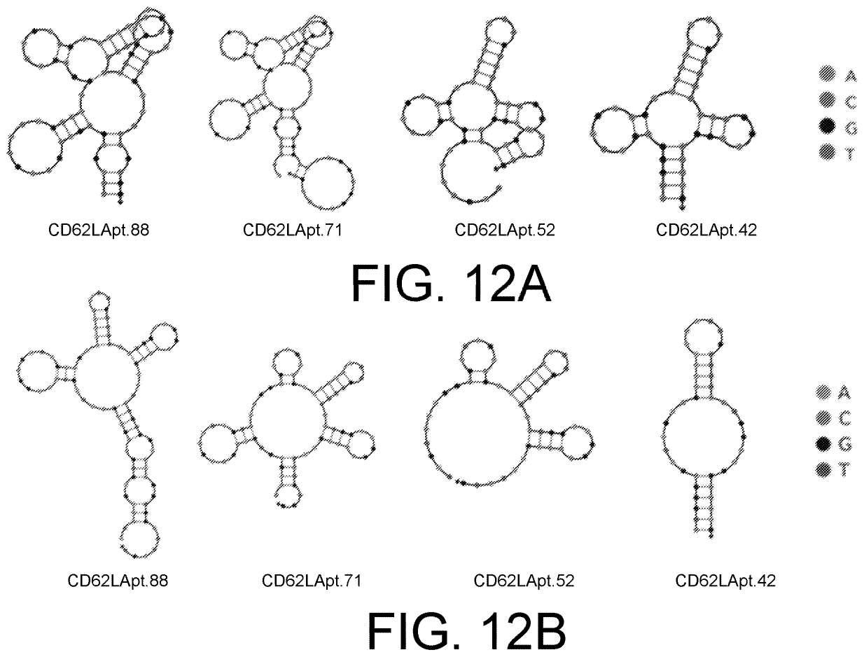

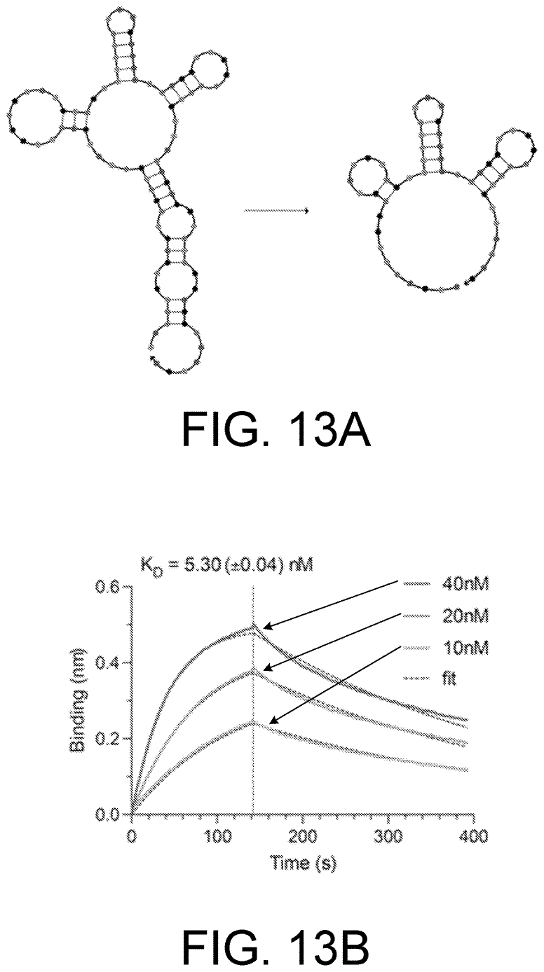

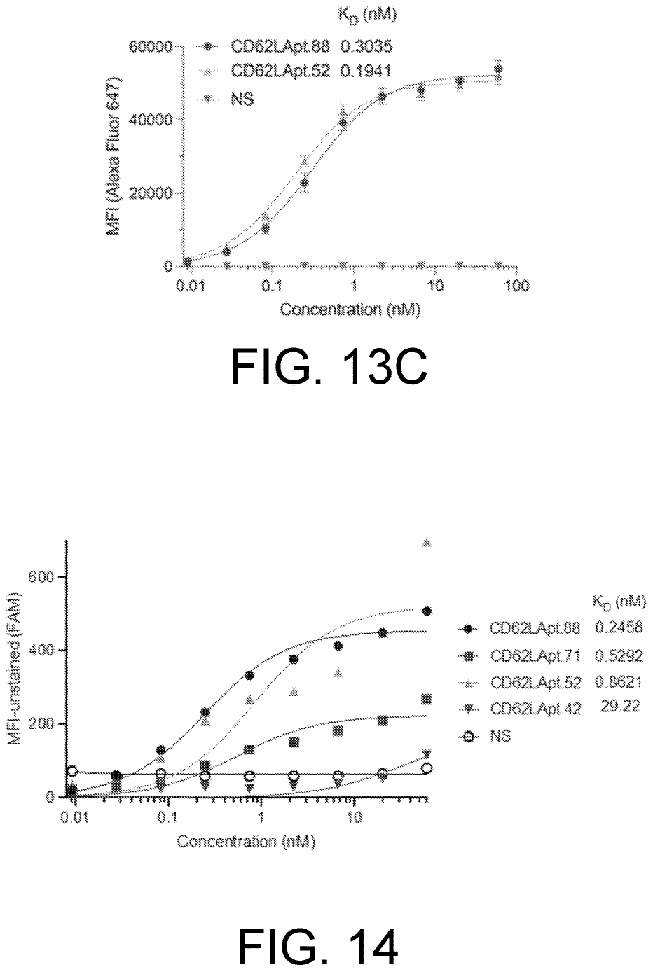

To reduce the downstream production cost and improve aptamer affinity and specificity, three aptamer truncations, CD62LApt.71, CD62LApt.52 and CD62LApt.42 (numbers denote the number of nucleotides in the sequences; FIGS. 12A, 12B) were designed, based on secondary structures predicted by both NUPACK 3 and the recently released NUPACK 4 that additionally considers coaxial and dangle stacking (Zadeh, J. N., et al., J. Comput. Chem., 2011. 32(1): p. 170-173). CD62LApt.88 was successively truncated to CD62LApt.71 by removing the lower stem-loops formed by the constant regions predicted by both NUPACK versions and to CD62LApt.52 by removing the constant regions (FIG. 13C). CD62LApt.42 was designed by removing sequences outside the three hairpins in the center, and then extending the stem at the base to form a stable structure. Binding of the fluorescein-labeled aptamer truncations was tested for binding to JRT.3-T3.5 cells by flow cytometry and observed high binding of CD62LApt.52 but not CD62LApt.42 to JRT.3-T3.5 (FIG. 14), indicating the predicted non-hairpin forming sequences contributes to a stable aptamer structure for binding to CD62L. While CD62LApt.71 appears to retain binding to JRT.3-T3.5 cells in this example, CD62LApt.52 was selected for further characterization because its shorter length offers a useful design control for downstream engineering.

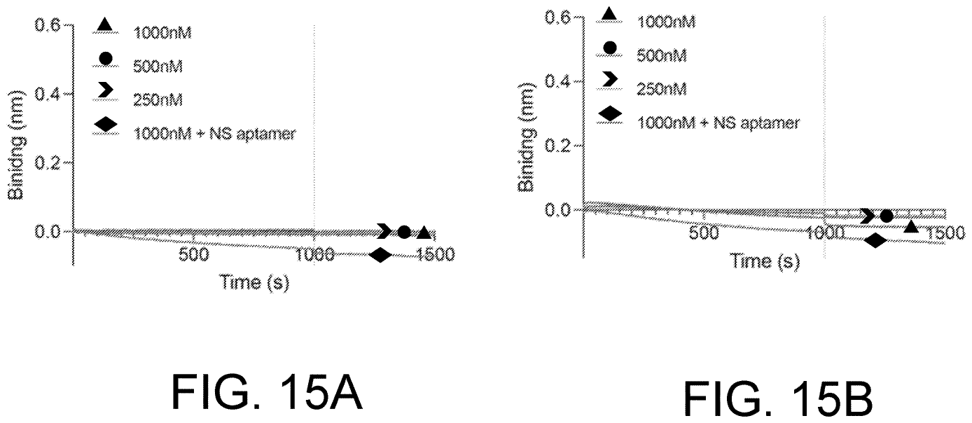

The binding affinity and kinetics of CD62LApt.52 to JRT.3-T3.5 cells and to recombinant human CD62L protein was characterized by flow cytometry and BLI, respectively. It was found that removing the constant regions has minimal impact on the aptamer's affinity, with K_{D}=0.19 nM to JRT.3-T3.5 cells and K_{D}=5.3 nM to recombinant human CD62L in this example (FIGS. 12B, 12C). Interestingly, while a high X² value and a low R² value were observed from the curve fitting for CD62LApt.88, indicating the binding is not characteristic of a 1:1 binding model, a low X² value and a high R² value approaching 1 were calculated for CD62LApt.52, with minimal changes in the calculated K_{D} (Table 6). This indicates that the truncation provides an improvement in specificity, as similarly reflected by the disappearance of the rapid onset of dissociation of CD62L protein from CD62LApt.52 during BLI. Binding of CD62LApt.52 to recombinant human CD62E/E-selectin and recombinant human CD62P/P-selectin proteins, which share conserved epitopes with CD62L, was tested. However, no noticeable binding of CD62LApt.52 to either CD62E or CD62P at up to 1000 nM was detected by BLI (FIGS. 15A, 15B).

TABLE 6
Binding Constants of Aptamers

| Aptamer | kₒₙ (10⁵ M⁻¹s⁻¹) | k_{dis} (10⁻³ s⁻¹) | K_{D} (nM) | χ² | R² |
|---|---|---|---|---|---|
| CD62LApt.88 | 5.250 (±0.193) | 2.576 (±0.049) | 4.907 (±0.203) | 2.0336 | 0.9029 |
| CD62LApt.61 | 2.988 (±0.057) | 1.549 (±0.018) | 5.814 (±0.125) | 0.3353 | 0.9898 |
| CD62LApt.52 | 5.543 (±0.043) | 2.937 (±0.011) | 5.297 (±0.045) | 0.0554 | 0.9959 |

Table 6 illustrates binding constants of CD62LApt.88, CD62LApt.61 and CD62LApt.52 to recombinant L-selectin measured by biolayer interferometry with immobilized aptamer on sensor.

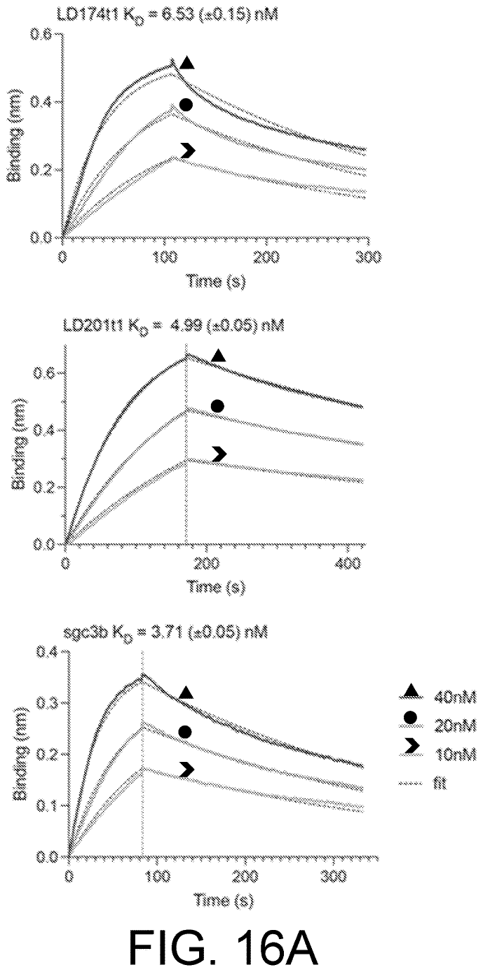

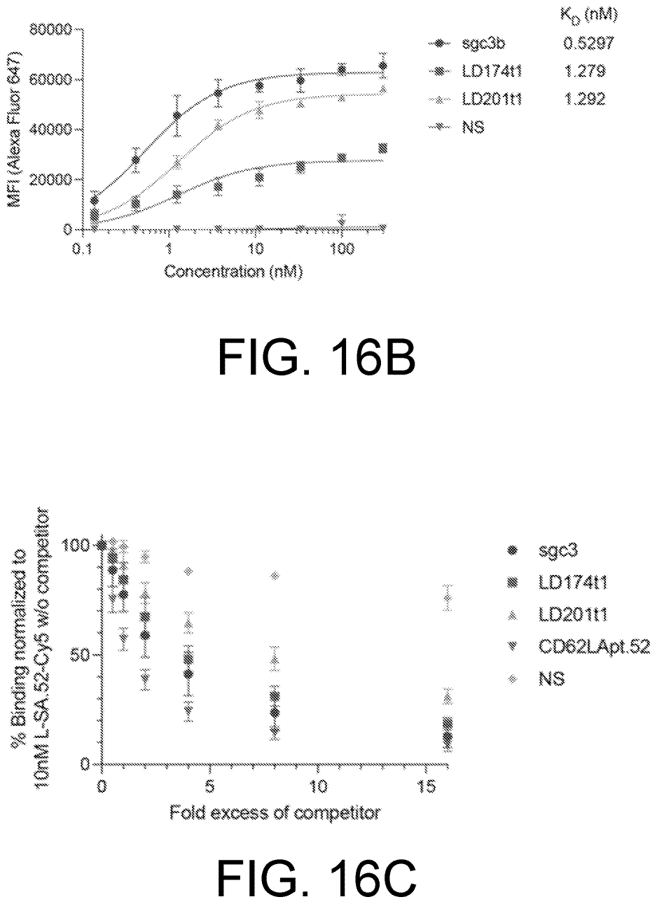

The truncated aptamer was compared with truncated versions of three previously reported CD62L aptamers, LD174t1, LD201t1 and sgc3b. It was verified that LD174t1, LD201t1 and sgc3b bind to recombinant human CD62L, with K_{D}=6.53 nM, K_{D}=4.99 nM, and K_{D}=3.71 nM, respectively, by BLI in the binding conditions (FIG. 16A). It was also verified that LD174t1, LD201t1 and sgc3b bind to JRT.3-T3.5 cells, with K_{D}=1.28 nM, K_{D}=1.29 nM, and K_{D}=0.53 nM, respectively, by flow cytometry in the binding conditions (FIG. 16B). LD174t1, LD201t1 and sgc3b were found to compete with CD62LApt.52 for binding to JRT.3-T3.5 cells in this example, although with slightly lower affinities (FIG. 16C). Collectively, these results additionally indicate the aptamers have high affinity and high specificity for CD62L.

### Reversal Agent Design for Rapid Aptamer Release

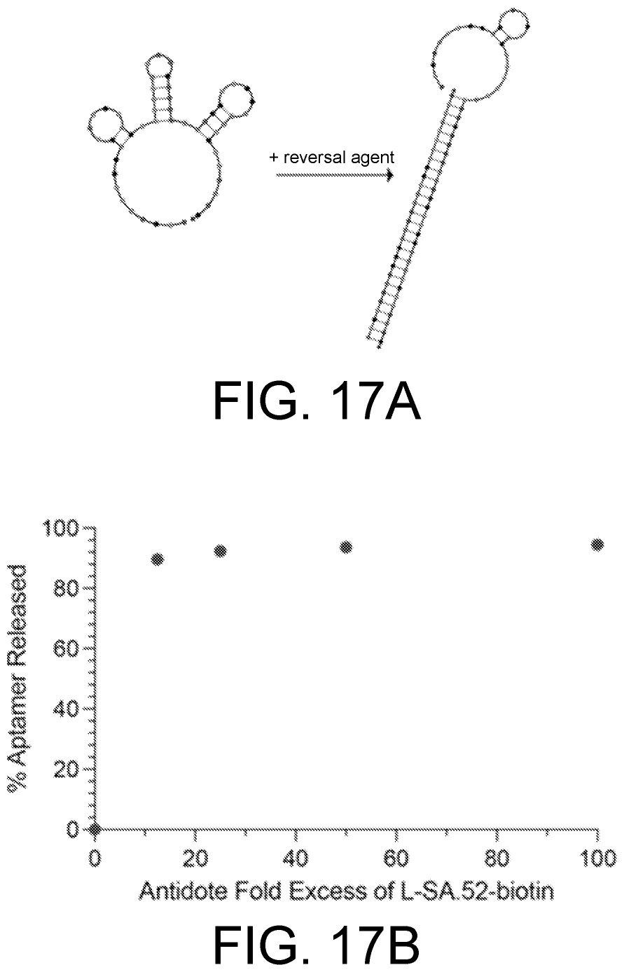

A major advantage of the use of aptamers in cell selection applications is the ability to rapidly and specifically reverse aptamer binding to cells after separation, rendering the selected cells label-free and available for further labeling and selection. The toehold-mediated strand displacement method was adopted for reversing aptamer binding in consideration of the specificity, low cost and gentle mechanics afforded by this method (Kacherovsky, N., et al., Nat. Biomed. Eng., 2019. 3(10): p. 783-795). While various toehold designs have been reported to improve displacement kinetics and efficiency, including by optimizing the toehold length and GC content, the multi-nucleotide sequence at the 3′ end of CD62LApt.52 can serve as a natural toehold for direct access of the reversal agent to disrupt the aptamer's secondary structures. A 28-nucleotide reversal agent that is predicted to extensively disrupt CD62LApt.52's structure from the 3′ end was designed (FIG. 17A). Addition of this reversal agent at >10-fold molar equivalent to CD62LApt.52 for 10 min at RT was found to reverse aptamer binding to J.RT3-T3.5 cells by >90% in this example (FIG. 17B).

### Aptamer Optimization for Cell Isolation

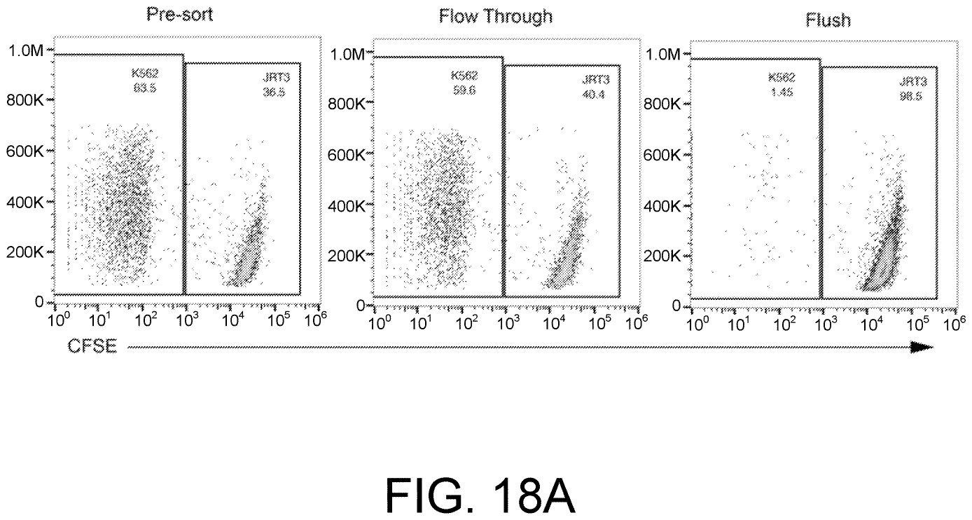

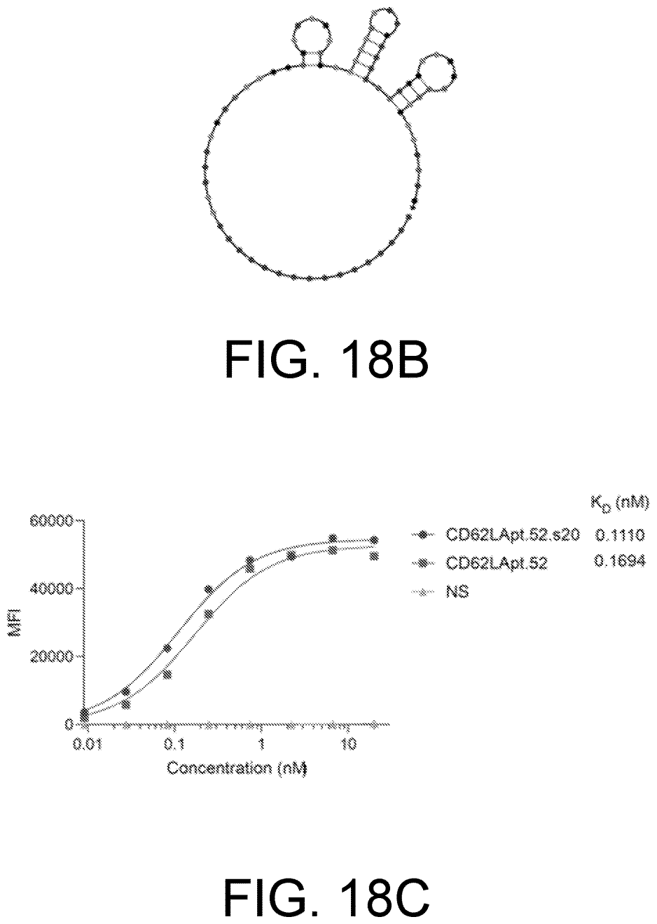

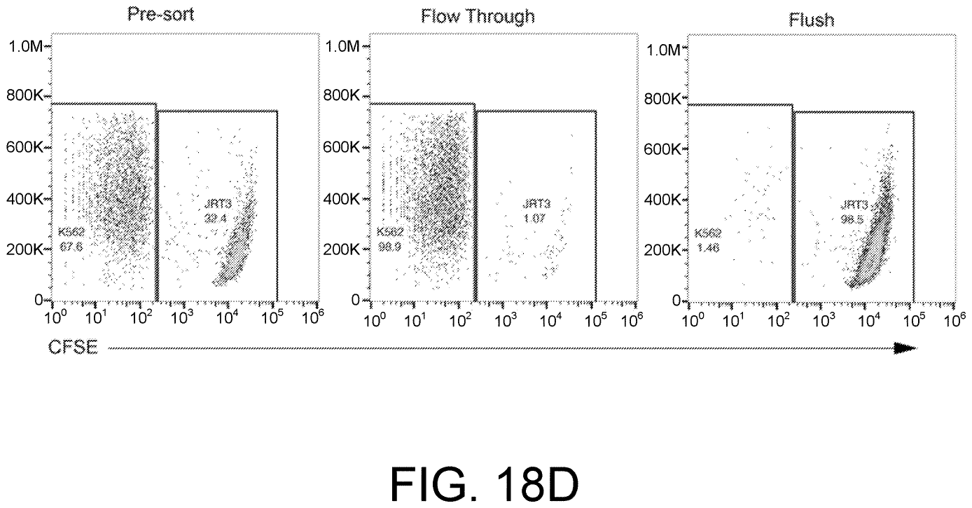

Towards the goal of achieving traceless selection of CD62L⁺CD8⁺ T cells, a completely synthetic magnetic selection system was implemented similarly as previously described (Kacherovsky, N., et al., Nat. Biomed. Eng., 2019. 3(10): p. 783-795). In this system, target cells are first labelled with biotinylated aptamers, after which the aptamer-labeled cells are incubated with anti-Biotin Microbeads for magnetic labelling. Subsequently, the labeled cells are applied onto a column under a magnetic field for magnetic separation. The ability of CD62LApt.52 to isolate CD62L⁺ cells was evaluated in a model mixture composed of CD62L⁺ J.RT3-T3.5 cells and CD62L⁻ K562 cells. While the CD62L⁺ J.RT3-T3.5 cells can be selected with high purity, a majority of the CD62L⁺ J.RT3-T3.5 cells were not retained on the column (FIG. 18A). A 20-nucleotide spacer at the 5′ end (CD62LApt.52.s20) was designed to extend the biotin moiety away from the aptamer structure and verified that the binding affinity of the aptamer is minimally affected by this design (FIGS. 18B, 18C). The incorporation of the spacer arm successfully retained CD62L⁺ J.RT3-T3.5 cells on the magnetic column and enabled selection with high yield and purity (FIG. 18D).

### Optimization of Aptamer Labelling and Releasing Conditions

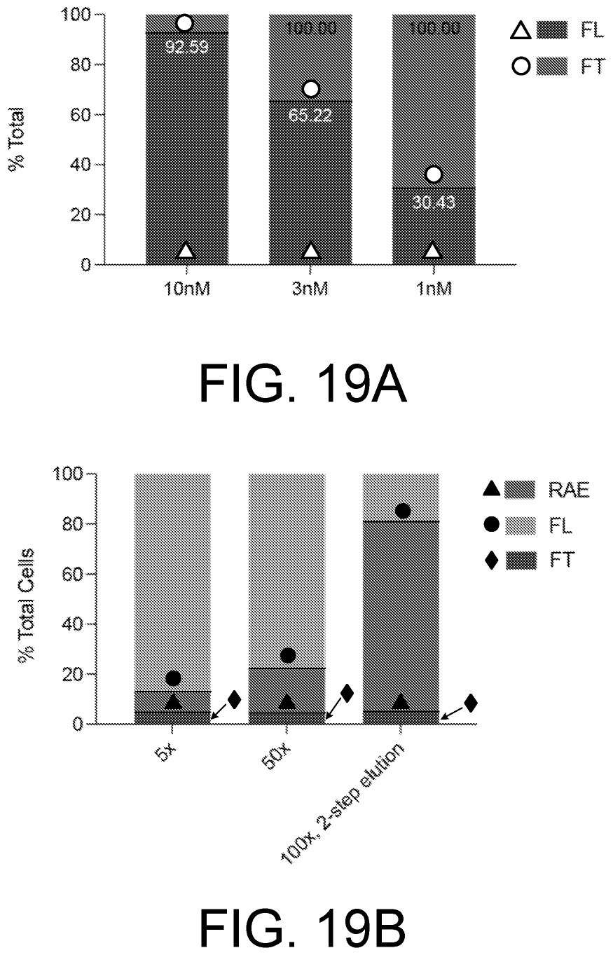

To streamline the selection process, it was evaluated whether biotinylated CD62LApt.52.s20 that are first immobilized on anti-Biotin Microbeads retain their ability to isolate J.RT3-T3.5 cells. A similar yield was observed with this approach compared to the previously used 2-step labeling approach. To reduce the selection cost by minimizing the amount of reagent necessary for cell selection, CD62LApt.52.s20 was titrated in the model system, and it was determined that the aptamer concentration can be reduced to 10 nM without affecting selection yield (FIG. 19A). Lastly, cell release from the aptamer-immobilized anti-Biotin Microbeads on magnetic columns was evaluated using the designed reversal agent. While high aptamer release was observed in 10 min at >10-fold reversal agent excess in a static well experiment in this example, low cell release from the column in the same conditions was observed (FIG. 19B). A 2-step elution strategy was designed, where additional reversal agent was passed into the column after the first elution and allowed to displace any remaining aptamers. The 2-step elution strategy resulted in an increased yield that is suitable for cell selection application.

### Pilot Cell Isolation Study

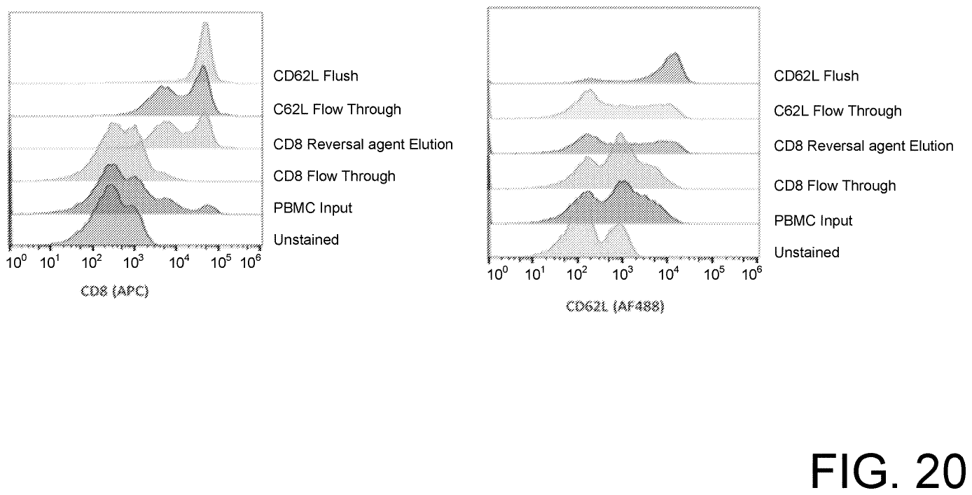

FIG. 20 illustrates example CD8 staining of cell populations during a double-aptamer isolation procedure and CD62L staining of cell populations during double-aptamer isolation procedure. The product (top histogram) has good purity as a CD8+CD62L+ population.

The CD62LApt.52.s20 aptamer was combined with the previously reported CD8 aptamer (CD8Apt) to develop a traceless and sequential method to select CD62L⁺CD8⁺ T cells from PBMCs. The aptamer-based, serial positive selection strategy was compared to the antibody-based, negative selection then-positive selection approach commonly used in the literature in terms of yield, purity and cell phenotype using cryopreserved PBMCs from 3 donors. Cryopreserved PBMCs were cultured overnight to allow for CD62L expression. CD8Apt was used to tracelessly isolate CD8⁺ T cells from PBMC as previously reported. The isolated CD8⁺ T cells were then incubated with biotinylated CD62LApt.52.s20 immobilized on anti-Biotin Microbeads to label CD62L⁺ cells. The cell mixture was applied onto a column under a magnetic field, and CD62L⁺CD8⁺ T cells were tracelessly eluted in two increments after incubation with 100-fold excess reversal agent for 10 min each. The column was then removed from the magnet and the remaining cells were collected using a column flush. Six fractions were collected after the serial positive selection process: CD8 Flow Through, CD8 Reversal Agent Elution (RAE), CD8 Flush (FL), CD62L Flow Through (FT), CD62L Reversal Agent Elution and CD62L Flush. For antibody-based isolation, PBMCs from the same donor were incubated with an antibody cocktail with antibodies against CD4, CD15, CD16, CD19, CD34, CD36, CD56, CD123, TCRγ/δ, and CD235a (Miltenyi Biotec). The antibody-labeled non-target cells were magnetically labeled with the CD8⁺ T Cell MicroBead Cocktail (Miltenyi Biotec) and depleted on a column under a magnetic field. The resulting unlabeled CD8⁺ cells were incubated with CD62L Microbeads (Miltenyi Biotec), applied on a column under a magnetic field, and the selected cells were flushed from the column. Four fractions were collected after the negative selection-then-positive selection process: CD8Ab Flow Through, CD8Ab Flush, CD62LAb Flow Through, and CD62LAb Flush. After selection, aptamer- and antibody-selected cells were analyzed by flow cytometry.

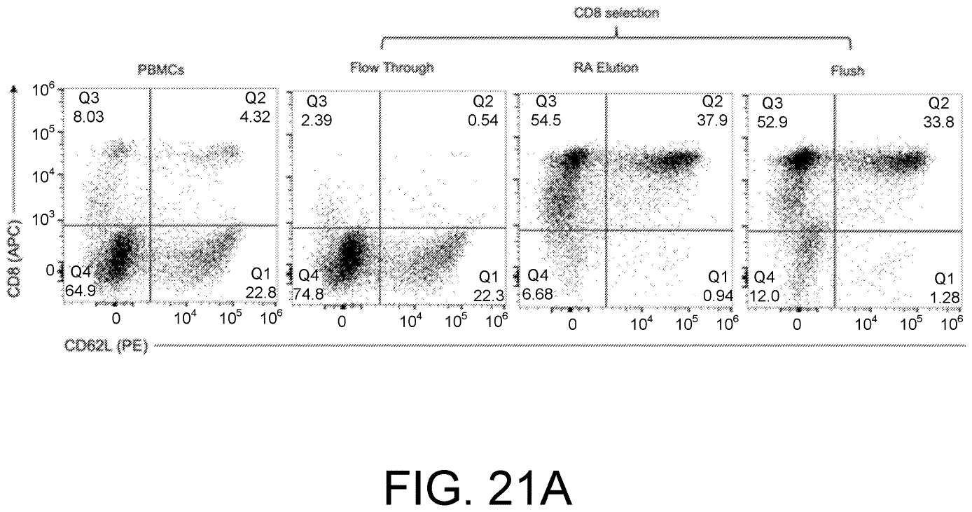

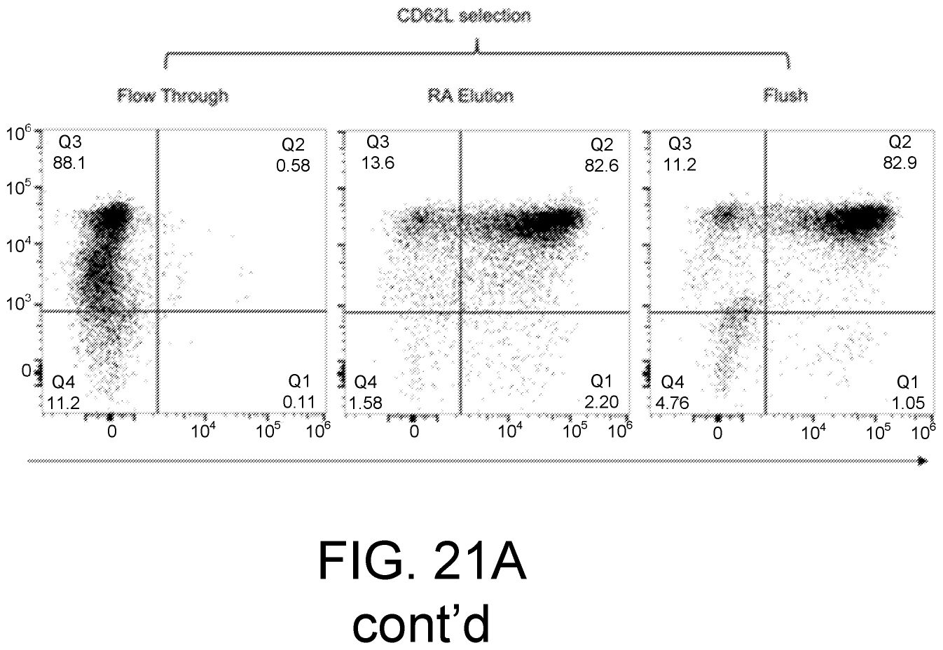

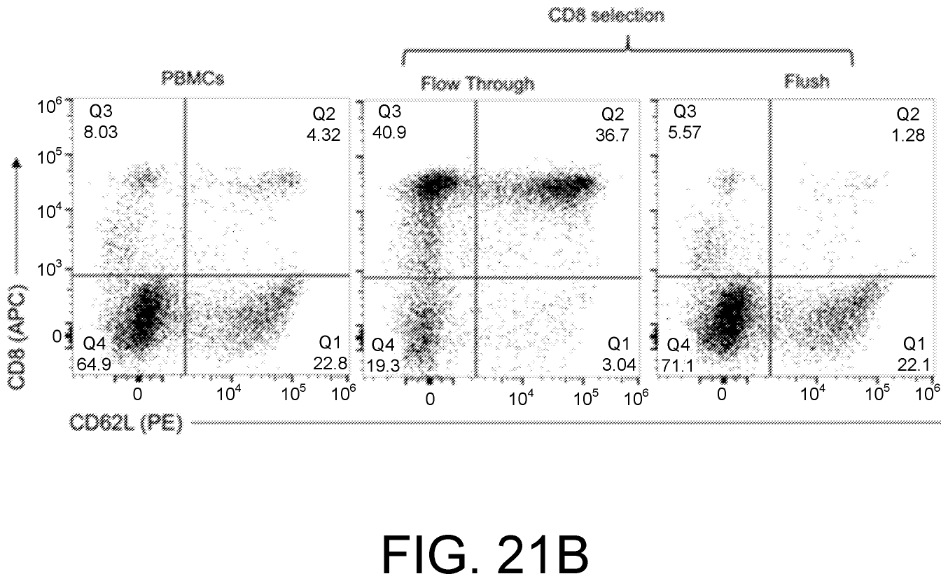

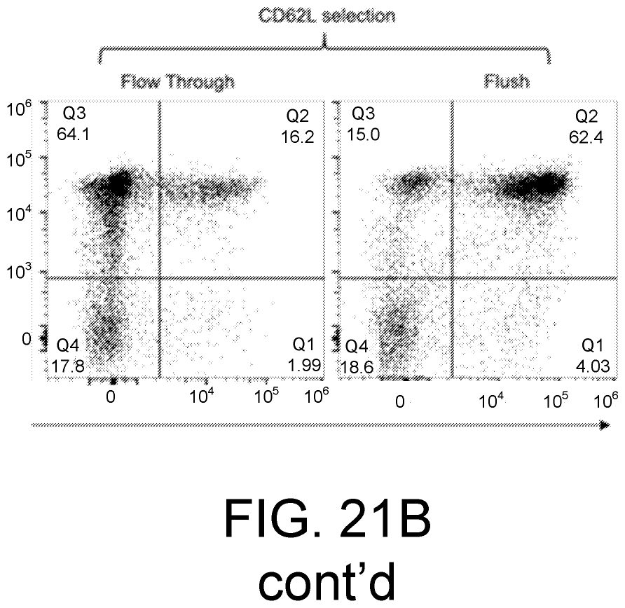

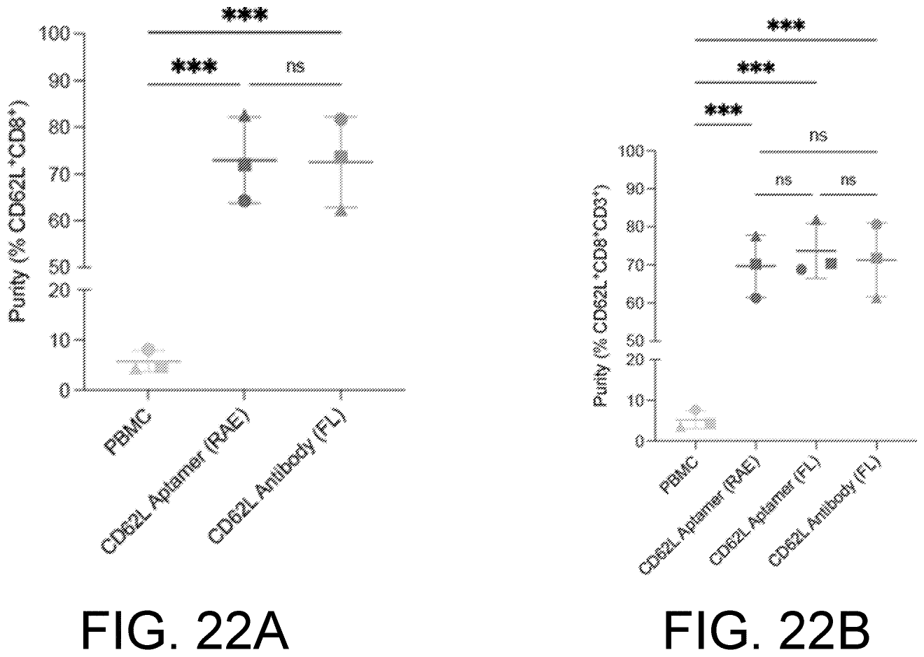

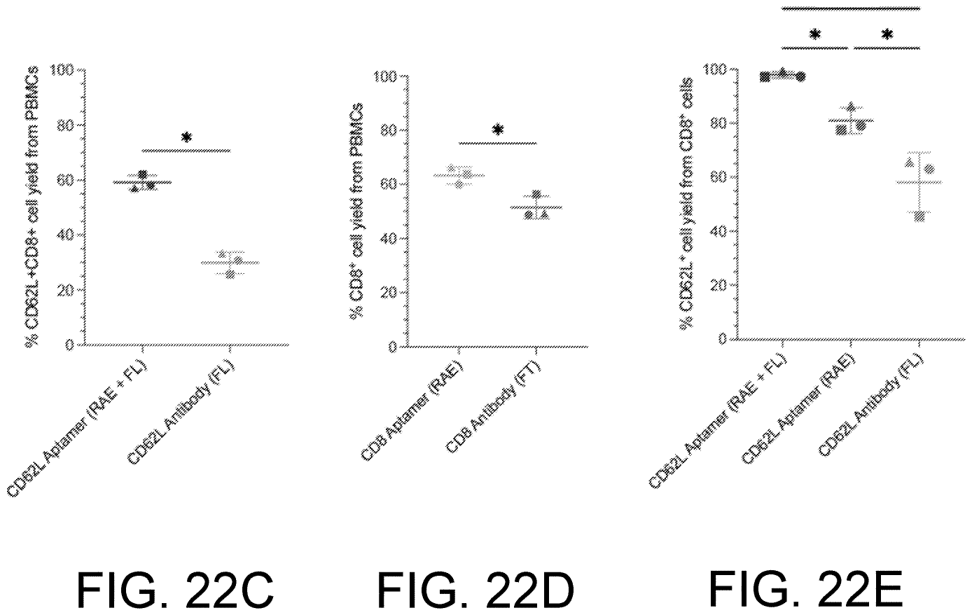

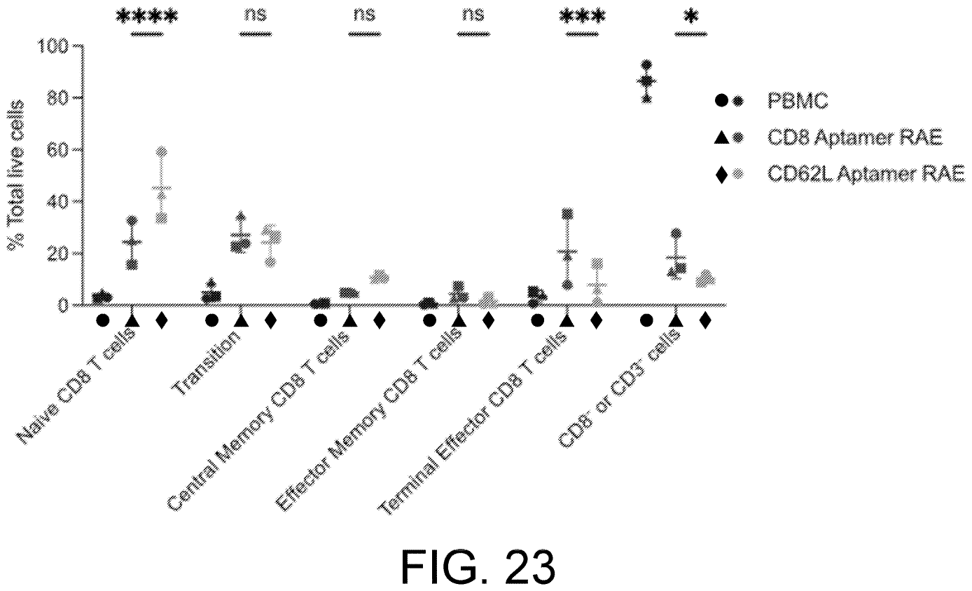

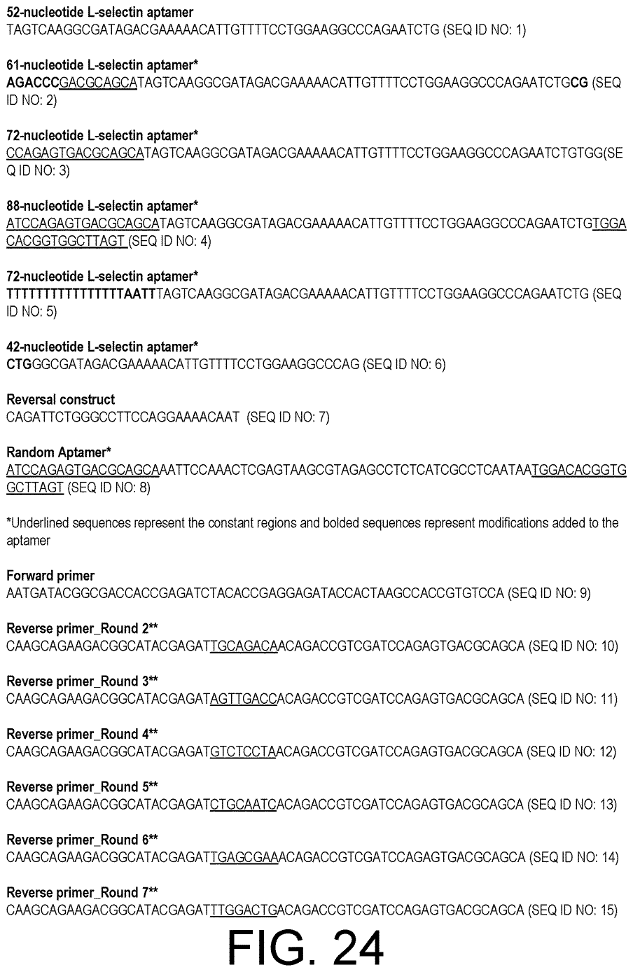

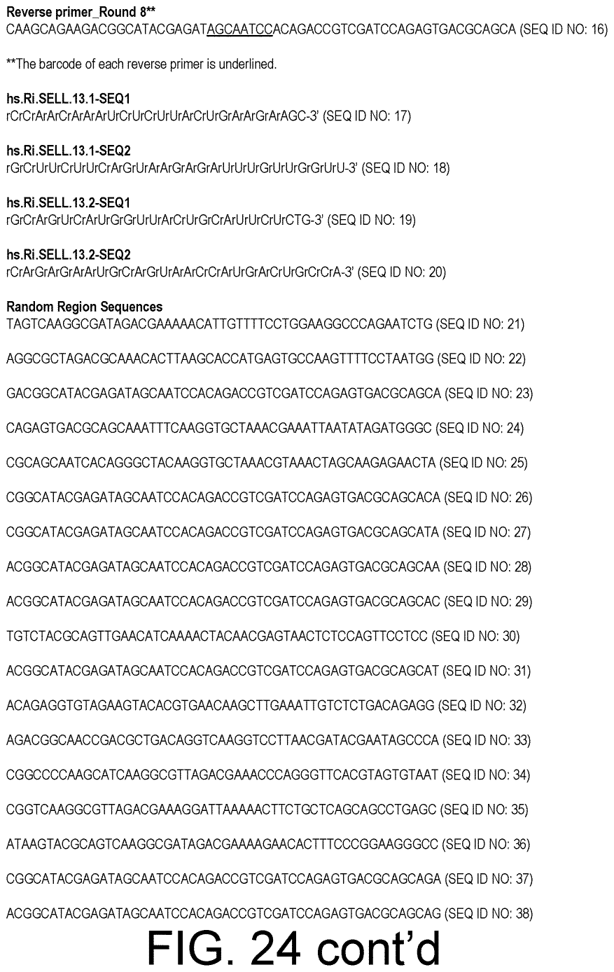

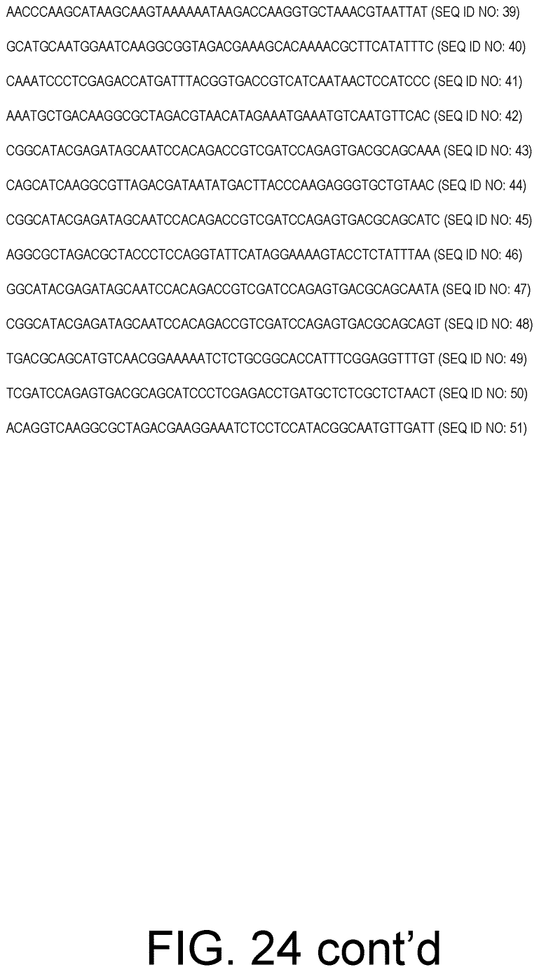

Using the aptamer-based strategy, near-complete selection of CD8⁺ cells in the first step was shown, followed by near-complete selection of CD62L⁺ in the second step (FIG. 21A). This corresponds to a significant enrichment of CD62L⁺CD8⁺ cells from 5.70% in the starting PBMCs to 72.97% in the CD62L RAE fraction and 75.03% in the CD62L Flush fraction in this example, comparable to the purity obtained by the traditionally used antibody-based method (FIG. 22A). The CD62L⁺CD8+CD3⁺ purity was analyzed to assess the specific enrichment in CD62L⁺CD8⁺ T cells. After aptamer-based CD8 selection and reversal agent elution, a small population of CD8¹⁰ cells was enriched, which includes NK cells and monocytes. However, this population was significantly reduced after aptamer-based CD62L selection (FIGS. 21A, 23). The resulting CD62L⁺CD8⁺CD3⁺ purity was 69.7% in the CD62L RAE fraction and 73.7% in the CD62L flush fraction in this example, which are comparable to the purity of the cells selected by the antibody-based method that had CD16 antibodies to deplete NK cells and monocytes (FIG. 22B). The results indicate that the serial positive selection method enables the selection of CD62L⁺CD8⁺ T cells with high purity that is at least comparable to the antibody-based method, while using minimal reagents to reduce manufacturing cost and variability.

The difference in yield between the aptamer-based method and the antibody-based method was compared by evaluating the percentage of target cells captured from the stating PBMCs. The aptamer-based method provides significantly improved yield over the antibody-based method, delivering an overall CD62L⁺CD8⁺ yield of 59.14% in this example, compared to 29.89% using the antibody-based method (FIG. 22C). Further analysis in each selection fraction revealed that the aptamer-based method offers a yield in both the CD8 selection step (63.3% for aptamer-based method vs 51.49% for antibody-based method; FIG. 22D) and the CD62L selection step (97.88% for aptamer-based method in this example vs 58.09% for antibody-based method; FIG. 22E). Even if only the RAE fraction is considered, which contains unlabeled cells that are available for re-labelling, the CD62L yield remains significantly higher for the aptamer-based method than the antibody-based method at 80.96%.

The phenotype of the isolated cells from the three separate donors was characterized (FIG. 23). Minimal difference was found between the compositions of the isolated cells, with naïve T cells being enriched most significantly, followed by transition and central memory T cells.

Notably, pre-clinical and clinical studies that investigated the potency of CAR T cells derived from central memory CD8⁺ T cells used CliniMACS CD4, CD14 and CD45RA antibodies for negative selection, followed by positive selection of CD62L⁺ cells using a custom-made, biotinylated CD62L monoclonal antibody (DREG-56) and anti-Biotin Microbeads. The comparison was based on a commercially available CD8 negative selection kit and commercially available CD62L Microbeads using the manufacturer's instructions to avoid experimental bias. As a result, the method enriches CD8⁺ T cells that express CD62L and depletes effector cells that do not express CD62L, while the previously reported method enriches exclusively central memory CD8 T cells. Given that CAR T cells derived from naïve and central memory CD8 T cells both showed improved survivals in in vivo models, the disclosed aptamer-based method provides an alternative strategy to manufacture CAR T cells from less differentiated CD8⁺ T cells. Despite using vastly distinct antibody sets, the observed yield from using the antibody-based method corroborates with the 25.2% reported by Terakura et al (Terakura, S., et al., Blood 2012, 119 (1), 72-82). In a clinical study that evaluated the potency of CAR T cells manufactured from defined CD4 and CD8 subsets, only 16 of 30 patients had sufficient CD8⁺ T_{CM} counts for selection using the protocols reported by Terakura et al (Terakura, S., et al., Blood 2012, 119 (1), 72-82; Turtle, C. J., et al., J Clin Invest 2016, 126 (6), 2123-38). By offering a significant improvement in yield while ensuring a label-free product, the disclosed aptamer-based, serial selection method addresses the limitations of traditional antibody-based selection strategies and facilitates the manufacturing of defined CAR T cells products for clinical evaluation.

### Discussion

This example describes the identification of a CD62L/L-Selectin aptamer that binds to both soluble and cell surface-expressed protein with nanomolar affinity. Application of the aptamers for isolation of CD62L⁺ T cells with high purity was demonstrated. In addition to the application for cell therapy manufacturing, CD62L aptamers have many other potential biomedical applications. For example, bivalent aptamers containing a CD62L-binding domain with a cancer cell-targeting domain have already been shown to mediate cell-cell interaction between T cells and cancer cells (Liu, X., et al., Small, 2011. 7(12): p. 1673-1682; Yang, Y., et al., ACS nano, 2020. 14(8): p. 9562-9571). Induction of cell-cell coupling followed by T cell activation induces cancer cell killing both in vitro and in vivo (Yang, Y., et al., ACS nano, 2020. 14(8): p. 9562-9571). Since CD62L facilitates immune cell infiltration into tissues, aptamer-based constructs that block CD62L binding can reduce immune responses after organ transplantation or in diseases such as diabetes. Antibody-based blocking of CD62L has been shown to prevent insulitis and diabetes in mouse models (Yang, X.-D., et al., Proc. Natl. Acad. Sci. U.S.A., 1993. 90(22): p. 10494-10498). In addition, lymphocyte entry to renal and cardiac transplants has been shown to be mediated by L-selectin (Turunen, J. P., et al., J. Exp. Med., 1995. 182(4): p. 1133-1141; Turunen, J. P., et al., Eur. J. Immunol., 1994. 24(5): p. 1130-1136). Reducing lymphocyte extravasation with CD62L-binding aptamers could therefore help reduce rejection after organ transplantation. Since the CD62L aptamers reported in this work bind to soluble L-Selectin, the aptamers could also be used in diagnostics. Soluble L-selectin levels are altered in diseases such as lupus, sepsis and diabetes-related coronary artery disease (Albertini, J.-P., et al., Diabetes Care, 1999. 22(12): p. 2044-2048; Font, J., et al., Clin. Exp. Immunol, 2000. 119(1): p. 169-174; Seidelin, J. B., et al., Intensive Care Med., 2002. 28(11): p. 1613-1618). The aptamers reported here could be applied as molecular recognition agents in point-of-care diagnostics for soluble L-selectin levels.

### EXAMPLE CLAUSES

1. A method of cell sorting, the method including:
   receiving a solution including a mixture of cells;
   exposing the solution to a construct including:
      an oligonucleotide that specifically binds L-selectin; and
      a tag or a solid support conjugated to the oligonucleotide; and
   isolating, from the solution, cells that express L-selectin by manipulating the tag or the solid support.
2. The method of clause 1, wherein the mixture of cells includes at least one of immune cells, red blood cells, epithelial cells, fat cells, muscle cells, or stem cells.
3. The method of clause 2, wherein the immune cells include at least one of naïve T cells, stem cell memory T cells, or central memory T cells.
4. The method of any of clauses 1-3, wherein the mixture of cells includes the cells that express L-selectin and cells that do not express L-selectin, and
wherein isolating, from the solution, the cells that express L-selectin includes isolating the cells that express L-selectin from the cells that do not express L-selectin.
5. The method of any of clauses 1-4, wherein the solid support includes paper, glass, a microbubble, a resin, a polymer, or a particle.
6. The method of any of clauses 1-5, wherein the solid support includes a microbubble, and
wherein isolating, from the solution, the cells that express L-selectin by manipulating the tag or the solid support includes:
   causing the microbubble to float to a surface of the solution; and
   aspirating, from the surface of the solution, the cells that express L-selectin.
7. The method of any of clauses 1-6, wherein the solid support includes a magnetic bead, and
wherein isolating, from the solution, the cells that express L-selectin by manipulating the tag or the solid support includes applying a magnetic field to the solution.
8. The method of any of clauses 1-7, wherein the tag includes at least one of a fluorophore, biotin, or a dye.
9. The method of clause 8, wherein the tag includes a fluorophore, and
wherein isolating, from the solution, the cells that express L-selectin by manipulating the tag or the solid support includes performing fluorescence activated cell sorting.
10. The method of any of clauses 1-9, wherein the cells that express L-selectin include CD62+ T cells.
11. The method of any of clauses 1-10, wherein the oligonucleotide includes a sequence having at least 70% sequence identity to the sequence as set forth in SEQ ID NO: 1.
12. The method of any of clauses 1-11, wherein the oligonucleotide includes a sequence having at least 70% sequence identity to the sequence as set forth in SEQ ID NO: 2.
13. The method of any of clauses 1-12, wherein the oligonucleotide includes a sequence having at least 70% sequence identity to the sequence as set forth in SEQ ID NO: 3.
14. The method of any of clauses 1-13, wherein the oligonucleotide includes a sequence having at least 70% sequence identity to the sequence as set forth in SEQ ID NO: 4.
15. The method of any of clauses 1-14, wherein the oligonucleotide includes a sequence having at least 70% sequence identity to the sequence as set forth in SEQ ID NO: 5.
16. The method of any of clauses 1-15, wherein the oligonucleotide includes a sequence having at least 70% sequence identity to the sequence as set forth in SEQ ID NO: 6.
17. The method of any of clauses 1-16, wherein the oligonucleotide includes a sequence having 80%, 95%, 97%, 98% or 99% sequence identity to the sequence as set forth in SEQ ID NO: 1.
18. The method of any of clauses 1-17, wherein the oligonucleotide includes a sequence having 80%, 95%, 97%, 98% or 99% sequence identity to the sequence as set forth in SEQ ID NO: 2.
19. The method of any of clauses 1-18, wherein the oligonucleotide includes a sequence having 80%, 95%, 97%, 98% or 99% sequence identity to the sequence as set forth in SEQ ID NO: 3.
20. The method of any of clauses 1-19, wherein the oligonucleotide includes a sequence having 80%, 95%, 97%, 98% or 99% sequence identity to the sequence as set forth in SEQ ID NO: 4.
21. The method of any of clauses 1-20, wherein the oligonucleotide includes a sequence having 80%, 95%, 97%, 98% or 99% sequence identity to the sequence as set forth in SEQ ID NO: 5.
22. The method of any of clauses 1-21, wherein the oligonucleotide includes a sequence having 80%, 95%, 97%, 98% or 99% sequence identity to the sequence as set forth in SEQ ID NO: 6.
23. The method of any of clauses 1-22, wherein the oligonucleotide has the sequence as set forth in SEQ ID NO: 1.
24. The method of any of clauses 1-23, wherein the oligonucleotide has the sequence as set forth in SEQ ID NO: 2.
25. The method of any of clauses 1-24, wherein the oligonucleotide has the sequence as set forth in SEQ ID NO: 3.
26. The method of any of clauses 1-25, wherein the oligonucleotide has the sequence as set forth in SEQ ID NO: 4.
27. The method of any of clauses 1-26, wherein the oligonucleotide has the sequence as set forth in SEQ ID NO: 5.
28. The method of any of clauses 1-27, wherein the oligonucleotide has the sequence as set forth in SEQ ID NO: 6.
29. The method of any of clauses 1-28, further including:
   receiving, from a subject, a biological sample including the mixture of cells; and
   producing the solution from the biological sample.
30. The method of clause 29, wherein the biological sample is a blood sample, a plasma sample, a serum sample, or a tissue sample.
31. The method of clause 29, wherein producing the solution includes at least one of:
   generating a cell suspension from the biological sample;
   filtering the biological sample;
   diluting the biological sample;
   washing the biological sample with a buffer; or
   performing lysis of one or more cells that do not express L-selectin.
32. The method of any of clauses 1-31, wherein the solution is a first solution, the construct is a first construct, the oligonucleotide is a first oligonucleotide, the tag is a first tag, and the solid support is a first solid support, the method further including:
   exposing a second solution including the cells that express L-selectin to a second construct including:
      a second oligonucleotide configured to specifically bind to a second target; and
      a second tag or a second solid support conjugated to the second oligonucleotide; and
   isolating, from the second solution, cells that express L-selectin and the second target by manipulating the second tag or the second solid support.
33. The method of clause 32, wherein the second target is CD8, and the isolated cells include CD62L+CD8+ T cells.
34. The method of any of clauses 1-33, further including:
   releasing the cells that express L-selectin from the construct.
35. The method of clause 34, wherein releasing the cells that express L-selectin from the construct includes:
   administering a reversal construct to the solution;
   changing a pH of the solution;
   changing a salt concentration of the solution;
   changing a temperature of the solution;
   applying a physical force to the solution; or
   administering a nuclease enzyme to the solution.
36. The method of clause 35, wherein the reversal construct includes an oligonucleotide including a sequence having at least 70% sequence identity to the sequence as set forth in SEQ ID NO: 7.
37. The method of clause 35 or 36, wherein the reversal construct includes an oligonucleotide including a sequence having at least 80%, 90%, 95%, 97%, or 99% sequence identity to the sequence set forth as SEQ ID NO: 7.
38. The method of any of clauses 35-37, wherein the reversal construct includes an oligonucleotide that has the sequence set forth as SEQ ID NO: 7.
39. An oligonucleotide that specifically binds L-selectin, the oligonucleotide including a sequence having at least 70% sequence identity to the sequence as set forth in SEQ ID NO: 1.
40. An oligonucleotide that specifically binds L-selectin, the oligonucleotide including a sequence having at least 70% sequence identity to the sequence as set forth in SEQ ID NO: 2.
41. An oligonucleotide that specifically binds L-selectin, the oligonucleotide including a sequence having at least 70% sequence identity to the sequence as set forth in SEQ ID NO: 3.
42. An oligonucleotide that specifically binds L-selectin, the oligonucleotide including a sequence having at least 70% sequence identity to the sequence as set forth in SEQ ID NO: 4.
43. An oligonucleotide that specifically binds L-selectin, the oligonucleotide including a sequence having at least 70% sequence identity to the sequence as set forth in SEQ ID NO: 5.
44. An oligonucleotide that specifically binds L-selectin, the oligonucleotide including a sequence having at least 70% sequence identity to the sequence as set forth in SEQ ID NO: 6.
45. An oligonucleotide that specifically binds L-selectin, the oligonucleotide including a sequence having at least 70% sequence identity to the sequence as set forth in SEQ ID NO: 1, SEQ ID NO: 2, SEQ ID NO: 3, SEQ ID NO: 4, SEQ ID NO: 5, or SEQ ID NO: 6.
46. The oligonucleotide of clause 45, wherein the oligonucleotide has a length of about 40 to about 90 nucleotides.
47. The oligonucleotide of clause 45 or 46, wherein the oligonucleotide has at least 80%, 95%, 97%, 98% or 99% sequence identity to the sequence set forth as SEQ ID NO: 1, SEQ ID NO: 2, SEQ ID NO: 3, SEQ ID NO: 4, SEQ ID NO: 5, or SEQ ID NO: 6.
48. The oligonucleotide of any of clauses 45-47, wherein the oligonucleotide has the sequence set forth as SEQ ID NO: 1, SEQ ID NO: 2, SEQ ID NO: 3, SEQ ID NO: 4, SEQ ID NO: 5, or SEQ ID NO: 6.
49. The oligonucleotide of any of clauses 45-48, wherein a binding affinity of the oligonucleotide to cells expressing L-selectin is in a range of about 0.005 nM to about 100 nM.
50. The oligonucleotide of any of clauses 45-49, wherein a binding affinity of the oligonucleotide to cells expressing L-selectin is in a range of about 0.01 nM to about 5 nM.
51. The oligonucleotide of any of clauses 45-50, wherein a binding affinity of the oligonucleotide to L-selectin is in a range of about 1 nM to about 1000 nM.
52. The oligonucleotide of any of clauses 45-51, wherein a binding affinity of the oligonucleotide to L-selectin is in a range of about 4 nM to about 100 nM.
53. The oligonucleotide of any of clauses 45-52, wherein the oligonucleotide binds soluble L-selectin and cell-expressed L-selectin.
54. The oligonucleotide of any of clauses 45-53, wherein the oligonucleotide is conjugated to a tag or a solid support.
55. The oligonucleotide of clause 54, wherein the tag includes a fluorophore, biotin, or a dye.
56. The oligonucleotide of clause 54 or 55, wherein the solid support includes a microbubble, a resin, a polymer, a particle, or a bead.
57. An aptamer including the oligonucleotide of any of clauses 39-56.
58. A composition including the oligonucleotide of any of clauses 39-56 and a pharmaceutically acceptable carrier.
59. A method including:
   administering, to a subject, a therapeutically effective dosage of the composition of clause 58.
60. A method of detecting a pathology of a subject, the method including:
   detecting cells expressing L-selectin using the oligonucleotide of any of clauses 39-56, wherein the cells are within the subject or within a biological sample obtained from the subject.
61. The method of clause 60, wherein detecting the cells expressing L-selectin includes:
   capturing, by the oligonucleotide bound to a surface, the cells expressing L-selectin;
   causing a detection antibody to bind to the cells expressing L-selectin, the detection antibody being bound to a tag; and
   outputting a detection signal associated with the tag.
62. The method of clause 61, wherein the tag is an enzyme, and outputting the detection signal includes introducing a detection substrate to the enzyme bound to the detection antibody.
63. The method of any of clauses 60-62, wherein detecting the cells expressing L-selectin bound to the oligonucleotide includes:
   detecting a signal output by a fluorophore conjugated to the oligonucleotide.
64. The method of any of clauses 60-63, wherein oligonucleotide is conjugated to a contrast agent or a radionuclide, and detecting cells expressing L-selectin includes:
   detecting the contrast agent or the radionuclide by imaging the cells.
65. A method of treating a subject in need thereof, the method including:
   administering a therapeutically effective amount of the oligonucleotide of any of clauses 39-56 to the subject in need thereof, thereby treating the subject.
66. The method of clause 65, wherein the subject is diagnosed with an autoimmune disorder, sepsis, organ transplant rejection, or cancer.
67. The method of clause 65 or 66, wherein administering the therapeutically effective amount of the oligonucleotide to the subject in need thereof includes administering the therapeutically effective amount of the oligonucleotide intravenously.
68. The method of any of clauses 65-67, wherein the oligonucleotide includes a multivalent oligonucleotide.
69. The method of clause 68, wherein the multivalent oligonucleotide includes an antigen targeting domain, the antigen targeting domain specifically binding an antigen expressed by a target cell of the subject.
70. The method of clause 69, wherein the antigen is a tumor-associated antigen or a tumor-specific antigen.
71. The method of any of clauses 65-70, wherein the oligonucleotide is conjugated to a drug.
72. The method of clause 71, wherein the drug is a cytotoxic agent.
73. The method of clause 72, wherein the cytotoxic agent includes actinomycin D, an alkylating agent, anthracycline, auristatin, calicheamicin, camptothecin, CC1065, colchicin, cyclophosphamide, cytarabine, cytochalasin B, daunorubicin, 1-dehydrotestosterone, dihydroxy anthracinedione, dolastatin, doxorubicin, duocarmycin, elinafide, emetine, ethidium bromide, etoposide, gramicidin D, glucocorticoids, lidocaine, maytansinoid, mithramycin, mitomycin, mitoxantrone, nemorubicin, PNU-159682, procaine, propranolol, puromycin, pyrrolobenzodiazepine, taxane, taxol, tenoposide, tetracaine, trichothecene, vinblastine, vinca alkaloid, or vincristine.
74. A method of treating a subject in need thereof, the method including:
   receiving a solution including a mixture of cells;
   exposing the solution to a construct including:
      an oligonucleotide that specifically binds L-selectin; and
      a tag or a solid support conjugated to the oligonucleotide; and removing, from the solution, cells that express L-selectin by manipulating the tag or the solid support; administering the solution to the subject, thereby treating the subject.
75. The method of clause 74, wherein the subject is diagnosed with a pathology associated with activated T cells.
76. The method of clause 74 or 75, wherein the subject is diagnosed with an autoimmune disorder, sepsis, or organ transplant rejection.
77. A construct including the oligonucleotide of any of clauses 39-56 conjugated to a tag, a solid support, or a linker.
78. The construct of clause 77, wherein the solid support includes a microbubble, a particle, or a bead.
79. The construct of clause 77 or 78, wherein the tag includes a fluorophore, biotin, or a dye.
80. The construct of any of clauses 77-79, including more than one oligonucleotide of any of clauses 39-56 conjugated to the tag, the solid support, or the linker.
81. The construct of any of clauses 77-80, including 2, 3, 4, 5, 6, 7, or 8 oligonucleotides of any of clauses 39-56 conjugated to the tag, the solid support, or the linker.
82. A system including the oligonucleotide of any of clauses 39-56 immobilized on a substrate.
83. The system of clause 82, wherein the substrate includes paper, glass, or a polymer.
84. A method of producing cells that express a chimeric antigen receptor (CAR), the method including:
receiving a solution including a mixture of cells;
exposing the solution to a construct including:
   an oligonucleotide configured to specifically bind L-selectin; and
   a tag or a solid support conjugated to the oligonucleotide; and
isolating, from the solution, cells that express L-selectin by manipulating the tag or the solid support;
transducing the cells that express L-selectin with a vector that encodes the CAR, thereby producing cells that express the CAR.
85. The method of clause 84, wherein the cells that express L-selectin include CD62L+ T cells.
86. The method of clause 84 or 85, wherein the cells that express L-selectin include at least one of naïve T cells, stem cell memory T cells, or central memory T cells.
87. A kit to isolate cells expressing L-selectin, the kit including:
   an oligonucleotide that specifically binds L-selectin.
88. The kit of clause 87, wherein the oligonucleotide includes a sequence having at least 70% sequence identity to the sequence as set forth in SEQ ID NO: 1, SEQ ID NO: 2, SEQ ID NO: 3, SEQ ID NO: 4, SEQ ID NO: 5, or SEQ ID NO: 6.
89. The kit of clause 87 or 88, wherein the oligonucleotide includes a sequence having 80%, 95%, 97%, 98% or 99% sequence identity to the sequence as set forth in SEQ ID NO: 1, SEQ ID NO: 2, SEQ ID NO: 3, SEQ ID NO: 4, SEQ ID NO: 5, or SEQ ID NO: 6.
90. The kit of any of clauses 87-89, wherein the oligonucleotide includes a sequence as set forth in SEQ ID NO: 1, SEQ ID NO: 2, SEQ ID NO: 3, SEQ ID NO: 4, SEQ ID NO: 5, or SEQ ID NO: 6.
91. The kit of any of clauses 87-90, wherein the oligonucleotide is conjugated to a tag or a solid support.
92. The kit of any of clauses 87-91, further including:
a reversal construct.
93. The kit of clause 92, wherein the reversal construct includes a sequence having at least 70% sequence identity to the sequence as set forth in SEQ ID NO: 7.
94. A reversal construct for an oligonucleotide, the reversal construct including a sequence having at least 70% sequence identity to SEQ ID NO: 7.
95. The reversal construct of clause 94, having at least 80% sequence identity to the sequence set forth as SEQ ID NO: 7.
96. The reversal construct of clause 94 or 95, having at least 90% sequence identity to the sequence set forth as SEQ ID NO: 7.
97. The reversal construct of any of clauses 94-96, having at least 95% sequence identity to the sequence set forth as SEQ ID NO: 7.
98. The reversal construct of any of clauses 94-97, having at least 97% sequence identity to the sequence set forth as SEQ ID NO: 7.
99. The reversal construct of any of clauses 94-98, having at least 98% sequence identity to the sequence set forth as SEQ ID NO: 7.
100. The reversal construct of any of clauses 94-99, having at least 99% sequence identity to the sequence set forth as SEQ ID NO: 7.
101. The reversal construct of any of clauses 94-100, having the sequence set forth as SEQ ID NO: 7.
102. The reversal construct of any of clauses 94-101, wherein the oligonucleotide specifically binds L-selectin and includes a sequence having at least 70% sequence identity to SEQ ID NO: 1.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Variants of the sequences disclosed and referenced herein are also included. Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.](1994), Meeting Date 1992, 111-20. Editor(s): Suhai, Sandor. Publisher: Plenum, New York, N.Y.. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of a binding domain (of, for example, a CAR binding domain or a nanoparticle selected cell targeting ligand) to its cognate binding molecule with an affinity or Ka (i.e., an equilibrium association constant of a particular binding interaction with units of 1/M) equal to or greater than 10⁵ M⁻¹, while not significantly associating with any other molecules or components in a relevant environment sample. “Specifically binds” is also referred to as “binds” herein. Binding domains may be classified as “high affinity” or “low affinity”. In particular embodiments, “high affinity” binding domains refer to those binding domains with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding domains refer to those binding domains with a Ka of up to 10⁷ M⁻¹, up to 10⁶ M⁻¹, up to 10⁵ M⁻¹. Alternatively, affinity may be defined as an equilibrium dissociation constant (Kd) of a particular binding interaction with units of M (e.g., 10⁻⁵ M to 10⁻¹³ M). In certain embodiments, a binding domain may have “enhanced affinity,” which refers to a selected or engineered binding domains with stronger binding to a cognate binding molecule than a wild type (or parent) binding domain. For example, enhanced affinity may be due to a Ka (equilibrium association constant) for the cognate binding molecule that is higher than the reference binding domain or due to a Kd (dissociation constant) for the cognate binding molecule that is less than that of the reference binding domain, or due to an off-rate (Koff) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

1-102. (canceled)

103. A method of cell sorting, the method comprising:
receiving a solution comprising a mixture of cells;
exposing the solution to a construct comprising:
an oligonucleotide that specifically binds soluble and cell-expressed L-selectin; and
a tag or a solid support conjugated to the oligonucleotide; and
isolating, from the solution, cells that express L-selectin by manipulating the tag or the solid support.

104. The method of claim 103, wherein the mixture of cells comprises at least one of immune cells, red blood cells, epithelial cells, fat cells, muscle cells, or stem cells; and/or
at least one of naïve T cells, stem cell memory T cells, or central memory T cells.

105. The method of claim 103, wherein the solid support comprises paper, glass, a microbubble, a resin, a polymer, or a particle; and/or
wherein the tag comprises at least one of a fluorophore, biotin, a dye, a chromophore tag, a quantum dot, a radionuclide, or an oligonucleotide.

106. The method of claim 103, wherein the oligonucleotide comprises a sequence having at least 70% sequence identity to the sequence as set forth in SEQ ID NO: 1.

107. The method of claim 103, wherein the oligonucleotide comprises a sequence having at least 80% sequence identity to the sequence as set forth in SEQ ID NO: 1.

108. The method of claim 103, wherein the oligonucleotide comprises a sequence having at least 90% sequence identity to the sequence as set forth in SEQ ID NO: 1.

109. The method of claim 103, further comprising:
releasing the cells that express L-selectin from the construct by:
administering a reversal construct to the solution;
changing a pH of the solution;
changing a salt concentration of the solution;
changing a temperature of the solution;
applying a physical force to the solution; or
administering a nuclease enzyme to the solution.

110. The method of claim 109, wherein the reversal construct comprises an oligonucleotide comprising a sequence having at least 70% sequence identity to the sequence as set forth in SEQ ID NO: 7.

111. The method of claim 109, wherein the reversal construct comprises an oligonucleotide comprising a sequence having at least 80% sequence identity to the sequence set forth as SEQ ID NO: 7.

112. An oligonucleotide that specifically binds L-selectin, the oligonucleotide comprising a sequence having at least 70% sequence identity to the sequence as set forth in SEQ ID NO: 1.

113. The oligonucleotide of claim 112, wherein the oligonucleotide comprises a sequence having at least 90% sequence identity to the sequence as set forth in SEQ ID NO: 1.

114. The oligonucleotide of claim 112, wherein the oligonucleotide has a length of about 40 to about 90 nucleotides.

115. The oligonucleotide of claim 112, wherein a binding affinity of the oligonucleotide to cells expressing L-selectin is in a range of about 0.005 nM to about 100 nM; and/or
wherein a binding affinity of the oligonucleotide to L-selectin is in a range of about 1 nM to about 1000 nM.

116. The oligonucleotide of claim 112, wherein a binding affinity of the oligonucleotide to cells expressing L-selectin is in a range of about 0.01 nM to about 5 nM; and/or
wherein a binding affinity of the oligonucleotide to L-selectin is in a range of about 4 nM to about 100 nM.

117. The oligonucleotide of claim 112, wherein the oligonucleotide is conjugated to a tag or a solid support.

118. The oligonucleotide of claim 112, wherein the oligonucleotide is conjugated to a cytotoxic drug.

119. A composition comprising the oligonucleotide of claim 112 and a pharmaceutically acceptable carrier.

120. A method of treating a subject in need thereof, the method comprising:
receiving a solution comprising a mixture of cells;
exposing the solution to a construct comprising:
an oligonucleotide that specifically binds soluble or cell-expressed L-selectin; and
a tag or a solid support conjugated to the oligonucleotide;
removing, from the solution, cells that express L-selectin by manipulating the tag or the solid support; and
administering the solution to the subject, thereby treating the subject.

121. The method of claim 120, wherein the subject is diagnosed with a pathology associated with activated T cells, an autoimmune disorder, sepsis, or organ transplant rejection.

122. The method of claim 120, wherein the oligonucleotide comprises a sequence having at least 70% sequence identity to the sequence as set forth in SEQ ID NO: 1.


SEQUENCE LISTING (51 sequences)

SEQ ID NO: 1 - DNA, 52 bp, synthetic construct
  source 1..52: mol_type = other DNA
tagtcaaggcgatagacgaaaaacattgttttcctggaaggcccagaatctg

SEQ ID NO: 2 - DNA, 69 bp, synthetic construct
  source 1..69: mol_type = other DNA
agacccgacgcagcatagtcaaggcgatagacgaaaaacattgttttcctggaaggcccagaatctgcg

SEQ ID NO: 3 - DNA, 71 bp, synthetic construct
  source 1..71: mol_type = other DNA
ccagagtgacgcagcatagtcaaggcgatagacgaaaaacattgttttcctggaaggcccagaatctgtgg

SEQ ID NO: 4 - DNA, 88 bp, synthetic construct
  source 1..88: mol_type = other DNA
atccagagtgacgcagcatagtcaaggcgatagacgaaaaacattgttttcctggaaggcccagaatctgtggacacggtggcttagt

SEQ ID NO: 5 - DNA, 72 bp, synthetic construct
  source 1..72: mol_type = other DNA
ttttttttttttttttaatttagtcaaggcgatagacgaaaaacattgttttcctggaaggcccagaatctg

SEQ ID NO: 6 - DNA, 42 bp, synthetic construct
  source 1..42: mol_type = other DNA
ctgggcgatagacgaaaaacattgttttcctggaaggcccag

SEQ ID NO: 7 - DNA, 28 bp, synthetic construct
  source 1..28: mol_type = other DNA
cagattctgggccttccaggaaaacaat

SEQ ID NO: 8 - DNA, 81 bp, synthetic construct
  source 1..81: mol_type = other DNA
atccagagtgacgcagcaaattccaaactcgagtaagcgtagagcctctcatcgcctcaataatggacacggtggcttagt

SEQ ID NO: 9 - DNA, 59 bp, synthetic construct
  source 1..59: mol_type = other DNA
aatgatacggcgaccaccgagatctacaccgaggagataccactaagccaccgtgtcca

SEQ ID NO: 10 - DNA, 61 bp, synthetic construct
  source 1..61: mol_type = other DNA
caagcagaagacggcatacgagattgcagacaacagaccgtcgatccagagtgacgcagca

SEQ ID NO: 11 - DNA, 61 bp, synthetic construct
  source 1..61: mol_type = other DNA
caagcagaagacggcatacgagatagttgaccacagaccgtcgatccagagtgacgcagca

SEQ ID NO: 12 - DNA, 61 bp, synthetic construct
  source 1..61: mol_type = other DNA
caagcagaagacggcatacgagatgtctcctaacagaccgtcgatccagagtgacgcagca

SEQ ID NO: 13 - DNA, 61 bp, synthetic construct
  source 1..61: mol_type = other DNA
caagcagaagacggcatacgagatctgcaatcacagaccgtcgatccagagtgacgcagca

SEQ ID NO: 14 - DNA, 61 bp, synthetic construct
  source 1..61: mol_type = other DNA
caagcagaagacggcatacgagattgagcgaaacagaccgtcgatccagagtgacgcagca

SEQ ID NO: 15 - DNA, 61 bp, synthetic construct
  source 1..61: mol_type = other DNA
caagcagaagacggcatacgagatttggactgacagaccgtcgatccagagtgacgcagca

SEQ ID NO: 16 - DNA, 61 bp, synthetic construct
  source 1..61: mol_type = other DNA
caagcagaagacggcatacgagatagcaatccacagaccgtcgatccagagtgacgcagca

SEQ ID NO: 17 - RNA, 25 nt, synthetic construct
  source 1..25: mol_type = other RNA
ccaacaaatctcttactgaagaagc

SEQ ID NO: 18 - RNA, 27 nt, synthetic construct
  source 1..27: mol_type = other RNA
gcttcttcagtaagagatttgttggtt

SEQ ID NO: 19 - RNA, 25 nt, synthetic construct
  source 1..25: mol_type = other RNA
gcagtcatggttactgcattctctg

SEQ ID NO: 20 - RNA, 27 nt, synthetic construct
  source 1..27: mol_type = other RNA
cagagaatgcagtaaccatgactgcca

SEQ ID NO: 21 - DNA, 52 bp, synthetic construct; identical to SEQ ID NO: 1
  source 1..52: mol_type = other DNA

SEQ ID NO: 22 - DNA, 52 bp, synthetic construct
  source 1..52: mol_type = other DNA
aggcgctagacgcaaacacttaagcaccatgagtgccaagttttcctaatgg

SEQ ID NO: 23 - DNA, 52 bp, synthetic construct
  source 1..52: mol_type = other DNA
gacggcatacgagatagcaatccacagaccgtcgatccagagtgacgcagca

SEQ ID NO: 24 - DNA, 52 bp, synthetic construct
  source 1..52: mol_type = other DNA
cagagtgacgcagcaaatttcaaggtgctaaacgaaattaatatagatgggc

SEQ ID NO: 25 - DNA, 52 bp, synthetic construct
  source 1..52: mol_type = other DNA
cgcagcaatcacagggctacaaggtgctaaacgtaaactagcaagagaacta

SEQ ID NO: 26 - DNA, 52 bp, synthetic construct
  source 1..52: mol_type = other DNA
cggcatacgagatagcaatccacagaccgtcgatccagagtgacgcagcaca

SEQ ID NO: 27 - DNA, 52 bp, synthetic construct
  source 1..52: mol_type = other DNA
cggcatacgagatagcaatccacagaccgtcgatccagagtgacgcagcata

SEQ ID NO: 28 - DNA, 52 bp, synthetic construct
  source 1..52: mol_type = other DNA
acggcatacgagatagcaatccacagaccgtcgatccagagtgacgcagcaa

SEQ ID NO: 29 - DNA, 52 bp, synthetic construct
  source 1..52: mol_type = other DNA
acggcatacgagatagcaatccacagaccgtcgatccagagtgacgcagcac

SEQ ID NO: 30 - DNA, 52 bp, synthetic construct
  source 1..52: mol_type = other DNA
tgtctacgcagttgaacatcaaaactacaacgagtaactctccagttcctcc

SEQ ID NO: 31 - DNA, 52 bp, synthetic construct
  source 1..52: mol_type = other DNA
acggcatacgagatagcaatccacagaccgtcgatccagagtgacgcagcat

SEQ ID NO: 32 - DNA, 52 bp, synthetic construct
  source 1..52: mol_type = other DNA
acagaggtgtagaagtacacgtgaacaagcttgaaattgtctctgacagagg

SEQ ID NO: 33 - DNA, 52 bp, synthetic construct
  source 1..52: mol_type = other DNA
agacggcaaccgacgctgacaggtcaaggtccttaacgatacgaatagccca

SEQ ID NO: 34 - DNA, 52 bp, synthetic construct
  source 1..52: mol_type = other DNA
cggccccaagcatcaaggcgttagacgaaacccagggttcacgtagtgtaat

SEQ ID NO: 35 - DNA, 52 bp, synthetic construct
  source 1..52: mol_type = other DNA
cggtcaaggcgttagacgaaaggattaaaaacttctgctcagcagcctgagc

SEQ ID NO: 36 - DNA, 52 bp, synthetic construct
  source 1..52: mol_type = other DNA
ataagtacgcagtcaaggcgatagacgaaaagaacactttcccggaagggcc

SEQ ID NO: 37 - DNA, 52 bp, synthetic construct
  source 1..52: mol_type = other DNA
cggcatacgagatagcaatccacagaccgtcgatccagagtgacgcagcaga

SEQ ID NO: 38 - DNA, 52 bp, synthetic construct
  source 1..52: mol_type = other DNA
acggcatacgagatagcaatccacagaccgtcgatccagagtgacgcagcag

SEQ ID NO: 39 - DNA, 52 bp, synthetic construct
  source 1..52: mol_type = other DNA
aacccaagcataagcaagtaaaaaataagaccaaggtgctaaacgtaattat

SEQ ID NO: 40 - DNA, 52 bp, synthetic construct
  source 1..52: mol_type = other DNA
gcatgcaatggaatcaaggcggtagacgaaagcacaaaacgcttcatatttc

SEQ ID NO: 41 - DNA, 52 bp, synthetic construct
  source 1..52: mol_type = other DNA
caaatccctcgagaccatgatttacggtgaccgtcatcaataactccatccc

SEQ ID NO: 42 - DNA, 52 bp, synthetic construct
  source 1..52: mol_type = other DNA
aaatgctgacaaggcgctagacgtaacatagaaatgaaatgtcaatgttcac

SEQ ID NO: 43 - DNA, 52 bp, synthetic construct
  source 1..52: mol_type = other DNA
cggcatacgagatagcaatccacagaccgtcgatccagagtgacgcagcaaa

SEQ ID NO: 44 - DNA, 52 bp, synthetic construct
  source 1..52: mol_type = other DNA
cagcatcaaggcgttagacgataatatgacttacccaagagggtgctgtaac

SEQ ID NO: 45 - DNA, 52 bp, synthetic construct
  source 1..52: mol_type = other DNA
cggcatacgagatagcaatccacagaccgtcgatccagagtgacgcagcatc

SEQ ID NO: 46 - DNA, 52 bp, synthetic construct
  source 1..52: mol_type = other DNA
aggcgctagacgctaccctccaggtattcataggaaaagtacctctatttaa

SEQ ID NO: 47 - DNA, 52 bp, synthetic construct
  source 1..52: mol_type = other DNA
ggcatacgagatagcaatccacagaccgtcgatccagagtgacgcagcaata

SEQ ID NO: 48 - DNA, 52 bp, synthetic construct
  source 1..52: mol_type = other DNA
cggcatacgagatagcaatccacagaccgtcgatccagagtgacgcagcagt

SEQ ID NO: 49 - DNA, 52 bp, synthetic construct
  source 1..52: mol_type = other DNA
tgacgcagcatgtcaacggaaaaatctctgcggcaccatttcggaggtttgt

SEQ ID NO: 50 - DNA, 52 bp, synthetic construct
  source 1..52: mol_type = other DNA
tcgatccagagtgacgcagcatccctcgagacctgatgctctcgctctaact

SEQ ID NO: 51 - DNA, 52 bp, synthetic construct
  source 1..52: mol_type = other DNA
acaggtcaaggcgctagacgaaggaaatctcctccatacggcaatgttgatt